(19) United States
(12) Patent Application Publication
(10) Pub. No.: US 2024/0376225 A1
(43) Pub. Date: Nov. 14, 2024

(54) CD21 ANTIBODIES AND USES THEREOF

(71) Applicants: Hulian GAO, Cambridge, MA (US); The Children's Medical Center Corporation, Boston, MA (US); PFIZER INC., New York, NY (US)

(72) Inventors: Hulian GAO, Cambridge, MA (US); Michael C. CARROLL, Boston, MA (US); Oleg Victorovich KOVALENKO, Cambridge, MA (US); Eric Matthew BENNETT, Cambridge, MA (US); Janet Elizabeth BUHLMANN, Cambridge, MA (US); Xianchun TANG, Cambridge, MA (US); Stefano Vincenzo GULLA, Sudbury, MA (US); Nathan HIGGINSON-SCOTT, Hingham, MA (US); Fridrik Jensen KARLSSON, Cambridge, MA (US); Dulce SOLER-FERRAN, Watertown, MA (US); Jessica Haewon MIN DEBARTOLO, Cambridge, MA (US)

(73) Assignees: The Children's Medical Center Corporation, Boston, MA (US); PFIZER INC., New York, NY (US)

(21) Appl. No.: 18/576,896

(22) PCT Filed: Jul. 6, 2022
(86) PCT No.: PCT/US2022/036166

§ 371 Date: Jan. 5, 2024

Related U.S. Application Data
(60) Provisional application No. 63/219,485, filed on Jul. 8, 2021.

(51) Int. Cl. C07K 16/28; A61P 37/06

(52) U.S. Cl. CPC C07K 16/2896; A61P 37/06; C07K 2317/21; C07K 2317/24; C07K 2317/622; C07K 2317/76

(57) ABSTRACT

Provided herein are anti-CD21 antibodies or antigen binding fragments thereof and their use in the treatment of autoimmune diseases or disorders.

### CROSS-REFERENCE TO RELATED APPLICATIONS

This application is a 35 U.S.C. § 371 National Phase Entry Application of International Application No. PCT/US2022/036166 filed Jul. 6, 2022, which designates the U.S. and claims benefit under 35 U.S.C. § 119 (e) to U.S. Provisional Application No. 63/219,485 filed Jul. 8, 2021, the contents of which are incorporated herein by reference in their entireties.

### GOVERNMENT SUPPORT

This invention was made with government support under Grant No. AI130307 and AI110164 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Sep. 16, 2022, is named 701039-099710WOPT.xml and is 137,000 bytes in size.

### TECHNICAL FIELD

The disclosure relates generally to anti-CD21 antibodies or antigen binding fragments thereof and their use in the treatment of autoimmune diseases or disorders.

### BACKGROUND

SLE is a systemic autoimmune B cell disorder characterized by excess immune complexes (IC) directed against highly conserved nuclear self-antigens such as ribonucleoproteins (RNP) and DNA (Shlomchik et al., 1994). A major goal in SLE research is to identify a therapy that “shuts-off” or ablates the spontaneous B cell response to self-antigen, particularly those responses that occur in germinal centers (GC), preventing the formation of affinity-matured, pathogenic autoantibodies. Within GC, activated B cells differentiate into high affinity isotype-switched plasma cells and form long-term memory B cells (Allen et al., 2007; Hauser et al., 2007a; Hauser et al., 2007b; MacLennan, 1994; Schwickert et al., 2007), events that correlate with poor prognostic outcomes when GC reactions are directed against auto-antigens. In humoral responses to infection, follicular dendritic cells (FDC) are important in retention of antigen and provide the necessary environment for GC development. FDC retain foreign antigen for extensive periods via complement receptor CR2. To limit expansion of auto-reactive B cell clones arising during somatic hypermutation (SHM), newly-formed GC B cells require contact with cognate antigen and the complement ligand C3d for their persistence (Fischer et al., 1998; Goodnow et al., 1995).

### SUMMARY

The methods and compositions provided herein are based, in part, on the optimization of anti-CD21 antibodies for binding CD21 and/or reducing B cell activation.

Accordingly, provided herein in one aspect is an antibody or antigen binding fragment that binds to CD21. In some embodiments of this aspect and all other aspects provided herein, the antibody or antigen fragment comprises a heavy chain variable region (V_{H}) comprising: (i) a CDR1 sequence differing by no more than four amino acids of an amino acid sequence selected from the group consisting of: SEQ ID NOs: 2, 30, 36, 44, 81, 84, 87, 90, 93, 96, and 99; (ii) a CDR2 sequence differing by no more than four amino acids of an amino acid sequence of SEQ ID NO: 3; and/or (iii) a CDR3 sequence differing by no more than four amino acids of an amino acid sequence selected from the group consisting of: SEQ ID NOs: 4, 37, and 51.

In one embodiment of this aspect and all other aspects provided herein, the antibody or antigen-binding fragment comprises a nanobody, an scFv, a monoclonal antibody, a humanized antibody, a human antibody, a recombinant antibody, a chimeric antibody, or a Fab fragment.

In another embodiment of this aspect and all other aspects provided herein, the antibody or antigen-binding fragment thereof further comprises a light chain variable region (V_{L}) comprising: (i) a CDR1 sequence differing by no more than four amino acids of an amino acid sequence selected from the group consisting of: SEQ ID NOs: 15, 33, 54, 59, 62, 65, (ii) a CDR2 sequence differing by no more than four amino acids of an amino acid sequence selected from the group consisting of: SEQ ID NOs: 16, and 68; and/or (iii) a CDR3 sequence differing by no more than four amino acids of an amino acid sequence selected from the group consisting of: SEQ ID NOs: 17, and 55.

In another embodiment of this aspect and all other aspects provided herein, the antibody or antigen-binding fragment further comprises at least one sequence from Table 8, 9 or 10.

In another embodiment of this aspect and all other aspects provided herein, the antibody or antigen binding fragment is a CD21 inhibitor or a CD21 neutralizing antibody. In another embodiment of this aspect and all other aspects provided herein, the antibody or antigen-binding fragment binds to or sterically blocks the C3 ligand binding site on CD21.

In another embodiment of this aspect and all other aspects provided herein, the antibody or antigen binding fragment reduces CD21 activity and/or B cell activation by at least 20% as compared to CD21 activity and/or B cell activation in the absence of the antibody or antigen-binding fragment. In another embodiment of this aspect and all other aspects provided herein, the antibody or antigen-binding fragment interferes with B lymphocyte co-receptor signaling; removes immune complexes from follicular dendritic cell (FDC), thereby removing availability of antigen and reducing cytokine secretion.

In another embodiment of this aspect and all other aspects provided herein, the antibody or antigen binding fragment disrupts binding of CD21 to its cognate ligand C3d.

In another embodiment of this aspect and all other aspects provided herein, the antibody or the antigen binding fragment is an isolated antibody or antigen binding fragment.

In another embodiment of this aspect and all other aspects provided herein, the antibody is a monoclonal antibody.

In another embodiment of this aspect and all other aspects provided herein, the antibody or antigen binding fragment is formulated as a pharmaceutical composition.

In another embodiment of this aspect and all other aspects provided herein, the antibody or antigen-binding fragment comprises a Vu chain comprising: (i) a CDR1 of SEQ ID NO: 2, (ii) a CDR2 of SEQ ID NO: 3, and (iii) a CDR3 of SEQ ID NO: 4, and optionally, a V_{L} chain comprising: (i) a CDR1 of SEQ ID NO: 15, (ii) a CDR2 of SEQ ID NO: 16, and (iii) a CDR3 of SEQ ID NO: 17.

In another embodiment of this aspect and all other aspects provided herein, the antibody comprises SEQ ID NOs: 2, 3 and 4, and optionally SEQ ID NOs: 33, 16 and 17.

In another embodiment of this aspect and all other aspects provided herein, the antibody comprises SEQ ID NOs: 30, 3 and 4, and optionally SEQ ID NOs: 33, 16 and 17.

In another embodiment of this aspect and all other aspects provided herein, the antibody comprises SEQ ID NOs: 30, 3, and 37, and optionally SEQ ID NOs: 15-17.

In another embodiment of this aspect and all other aspects provided herein, the antibody comprises SEQ ID NOs: 30, 3, and 37, and optionally SEQ ID NOs: 33, 16 and 17.

In another embodiment of this aspect and all other aspects provided herein, the antibody comprises SEQ ID NOs: 36, 3 and 37, and optionally SEQ ID NOs: 33, 16 and 17.

In another embodiment of this aspect and all other aspects provided herein, the antibody comprises SEQ ID NOs: 44, 3, and 37, and optionally SEQ ID NOs: 15-17.

In another embodiment of this aspect and all other aspects provided herein, the antibody comprises SEQ ID NOs: 44, 3, and 37, and optionally SEQ ID NOs: 33, 16 and 17.

In another embodiment of this aspect and all other aspects provided herein, the antibody comprises SEQ ID NOs: 2, 3, and 37, and optionally SEQ ID NOs: 15-17.

In another embodiment of this aspect and all other aspects provided herein, the antibody comprises SEQ ID NOs: 2, 3, and 37, and optionally SEQ ID NOs: 33, 16 and 17.

In another embodiment of this aspect and all other aspects provided herein, the antibody comprises SEQ ID NOs: 2, 3, and 51, and optionally SEQ ID NOs: 54, 16, and 55.

In another embodiment of this aspect and all other aspects provided herein, the antibody comprises SEQ ID NOs: 2, 3, and 51 and optionally SEQ ID NOs: 59, 16 and 55.

In another embodiment of this aspect and all other aspects provided herein, the antibody comprises SEQ ID NOs: 2, 3, and 51 and optionally SEQ ID NOs: 62, 16, and 55.

In another embodiment of this aspect and all other aspects provided herein, the antibody comprises SEQ ID NOs: 2, 3, and 51 and optionally SEQ ID NOs: 65, 16, and 55.

In another embodiment of this aspect and all other aspects provided herein, the antibody comprises SEQ ID NOs: 2, 3, and 51 and optionally SEQ ID NOs: 54, 68, and 55.

In another embodiment of this aspect and all other aspects provided herein, the antibody comprises SEQ ID NOs: 2, 3, and 51 and optionally SEQ ID NOs: 59, 68, and 55.

In another embodiment of this aspect and all other aspects provided herein, the antibody comprises SEQ ID NOs: 2, 3, and 51 and optionally SEQ ID NOs: 62, 68 and 55.

In another embodiment of this aspect and all other aspects provided herein, the antibody comprises SEQ ID NOs: 2, 3, and 51 and optionally SEQ ID NOs: 62, 68 and 55.

In another embodiment of this aspect and all other aspects provided herein, the antibody comprises SEQ ID NOs: 2, 3, and 51 and optionally SEQ ID NOs: 65, 68 and 55.

In another embodiment of this aspect and all other aspects provided herein, the antibody comprises SEQ ID NOs: 44, 3, and 51 and optionally SEQ ID NOs: 54, 68 and 55.

In another embodiment of this aspect and all other aspects provided herein, the antibody comprises SEQ ID NOs: 81, 3, 51 and optionally SEQ ID NOs: 54, 68, and 55.

In another embodiment of this aspect and all other aspects provided herein, the antibody comprises SEQ ID NOs: 84, 3 and 51 and optionally SEQ ID NOs: 54, 68, and 55.

In another embodiment of this aspect and all other aspects provided herein, the antibody comprises SEQ ID NOs: 87, 3 and 51 and optionally SEQ ID NOs: 54, 68, and 55.

In another embodiment of this aspect and all other aspects provided herein, the antibody comprises SEQ ID NOs: 90, 3, and 51 and optionally SEQ ID NOs: 54, 68, and 55.

In another embodiment of this aspect and all other aspects provided herein, the antibody comprises SEQ ID NOs: 93, 3, and 51 and optionally SEQ ID NOs: 54, 68, and 55.

In another embodiment of this aspect and all other aspects provided herein, the antibody comprises SEQ ID NOs: 96, 3, and 51 and optionally SEQ ID NOs: 54, 68, and 55.

In another embodiment of this aspect and all other aspects provided herein, the antibody comprises SEQ ID NOs: 99, 3, and 51 and optionally SEQ ID NOs: 54, 68, and 55.

Another aspect provided herein relates to an antibody or antigen binding fragment that binds to CD21 and comprises a variable heavy (V_{H}) chain comprising: (i) a CDRI sequence comprising an amino acid sequence that differs by no more than four amino acids from G-G-X₁-X₂-X₃-X₄-X₅-A-I-S (SEQ ID NO: 102), wherein X₁ is T or E; X₂ is F or L; X₃ is S, or Q; X₄ is T, D, or S; X₅ is Y or L; (ii) a CDR2 sequence comprising an amino acid sequence that differs by no more than four amino acids from RIIPILGIAN YAQKFQG (SEQ ID NO: 3); and (iii) a CDR3 sequence comprising comprising an amino acid sequence that differs by no more that four amino acids from E-X₆-D-X₇-S-G-Y-Y-Q-D (SEQ ID NO: 103), wherein X₆ is D or E; X₇ is S or I.

In one embodiment of this aspect and all other aspects provided herein, the antibody or antiben-binding fragment further comprises a light variable region (V_{L}) comprising: (i) a CDR1 sequence comprising comprising an amino acid sequence that differs by no more than four amino acids from R-A-G-Q-X₈-I-X₉-X₁₀-Y-L-A (SEQ ID NO: 104), wherein X₈ is S or K; X₉ is N or S; X₁₀ is S, H, or R; (ii) a CDR2 sequence comprising comprising an amino acid sequence that differs by no more than four amino acids from D-A-S-X₁₁-R-A-T (SEQ ID NO: 105), wherein X₁₁ is S or T; and (iii) a CDR₃ sequence comprising comprising an amino acid sequence that differs by no more than four amino acids from Q-Q-Y-E-Y-W-Y-X₁₂ (SEQ ID NO: 106), wherein X₁₂ is S or T.

In another embodiment of this aspect and all other aspects provided herein, the antibody or the antigen-binding fragment further comprises at least one amino acid sequence that differs by no more than four amino acids from at least one amino acid sequence from Table 8, 9 and/or 10.

In another embodiment of this aspect and all other aspects provided herein, the antibody or antigen-binding fragment comprises a CD21 inhibitor or a CD21 neutralizing antibody.

In another embodiment of this aspect and all other aspects provided herein, the antibody or antigen-binding fragment binds to or sterically blocks the C3 ligand binding site on CD21.

In another embodiment of this aspect and all other aspects provided herein, the antibody or antigen binding fragment reduces CD21 activity and/or B cell activation by at least 20% as compared to CD21 activity and/or B cell activation in the absence of the antibody or antigen-binding fragment. In another embodiment of this aspect and all other aspects provided herein, the antibody or antigen-binding fragment interferes with B lymphocyte co-receptor signaling; removes immune complexes from follicular dendritic cell (FDC), thereby removing availability of antigen and reducing cytokine secretion.

Another aspect provided herein relates to a pharmaceutical composition comprising a therapeutically effective amount of an antibody or antigen-binding fragment as described herein and a pharmaceutically acceptable carrier or excipient.

In another aspect, provided herein is a cell comprising an antibody or antigen binding fragment described herein.

In yet another aspect, provided herein is a polynucleotide comprising a nucleotide sequence encoding an antibody or antigen binding fragment described herein.

In another aspect, provided herein is a pharmaceutical composition comprising a polynucleotide comprising a nucleotide sequence encoding an antibody or antigen binding fragment described herein and a pharmaceutically acceptable carrier or excipient.

In still another aspect, provided herein is a cell comprising a polynucleotide comprising a nucleotide sequence encoding an antibody or antigen binding fragment described herein.

In yet still another, provided herein is a kit comprising a polynucleotide comprising a nucleotide sequence encoding an antibody or antigen binding fragment described herein.

Another aspect provided herein relates to a method for reducing at least one symptom of an autoimmune disease or disorder, the method comprising administering a therapeutically effective amount of the antibody or antigen-binding fragment as described herein or a polynucleotide encoding the antibody or antigen-binding fragment.

In one embodiment of this aspect and all other aspects provided herein, the autoimmune disease or disorder comprises systemic lupus erythematosus (SLE).

In another embodiment of this aspect and all other aspects provided herein, the at least one symptom of SLE comprises lupus nephritis, malar rash, discoid rash, butterfly rash, photosensitivity, oral ulcers, arthritis, serositis, pleuritis, pericarditis, proteinuria, seizures, psychosis, hemolytic anemia, leukopenia, lymphopenia, or thrombocytopenia. In another embodiment of this aspect and all other aspects provided herein, treatment of SLE comprises comprise reduction of autoreactive B cells, which in turn reduces the production of autoantibodies.

In another embodiment of this aspect and all other aspects provided herein, the at least one symptom of SLE comprises a clinical measure of SLE selected from the group consisting of: positive lupus erythematosus cell preparation, anti-DNA antibody to native DNA ratio, and anti-SM nuclear antigen antibodies.

In another embodiment of this aspect and all other aspects provided herein, the antibody or antigen-binding fragment binds to or sterically blocks the C3 ligand binding site on CD21.

In another embodiment of this aspect and all other aspects provided herein, the antibody or antigen binding fragment reduces CD21 activity and/or B cell activation by at least 20% as compared to CD21 activity and/or B cell activation in the absence of the antibody or antigen-binding fragment. In another embodiment of this aspect and all other aspects provided herein, the antibody or antigen-binding fragment interferes with B lymphocyte co-receptor signaling; removes immune complexes from follicular dendritic cell (FDC), thereby removing availability of antigen and reducing cytokine secretion.

Another aspect provided herein relates to a method for reducing CD21 activity or CD21/C3d mediated B cell activation, the method comprising: administering to a B cell an antibody or antigen-binding fragment described herein or a polynucleotide encoding the antibody or antigen-binding fragment, thereby reducing CD21 activity and/or CD21 mediated B cell activation.

In one embodiment of this aspect and all other aspects provided herein, the administering to the cell occurs in vitro or in vivo.

Another aspect provided herein relates to a kit comprising an antibody or antigen-binding fragment as described herein or a pharmaceutical composition thereof and instructions for use therefor.

Another aspect provided herein relates to any composition as described herein for use in the treatment of an autoimmune disease or disorder, or at least one symptom thereof in a subject.

### BRIEF DESCRIPTION OF THE DRAWINGS

FIG. 1 is a graph showing data that Ab15 binds to human and cynomolgus CD21 protein by ELISA.

FIG. 2 shows that Ab 15 binds to cell-surface human and cyno CD21 as measured by flow cytometry. An 8-point, 3-fold dilution series was made with Ab15 and binding to human Raji B cell (opened circle) or CHO cells overexpressing cyno CD21 (filled square) was evaluated. Dose-dependent binding is seen with both cells, indicating that Ab15 binds to both human and cyno CD21.

FIG. 3 Ab15 neutralizes C3d binding to cells expressing human or cyno CD21.

FIGS. 4A-4B Representative Biacore sensorgrams of Ab15 binding to human and cyno CD21 proteins. The KD of Ab 15 binding to human or cyno CD21 was determined to be 15.59 nM (FIG. 4A) and 5.77 nM (FIG. 4B), respectively.

FIG. 5 is a sequence alignment of affinity-improved Ab194 and Ab204 to parental Ab15 and its germlined derivative, Ab89. The differences in the amino acid sequences are shaded.

FIG. 6 is a sequence alignment of antibodies Ab416-Ab424 with Ab15/89, Ab194 and Ab204 (SEQ ID NOs: 107-113).

FIG. 7 is a sequence alignment of Ab626-Ab633 with Ab423 (SEQ ID NOs: 114-120).

FIG. 8 is a sequence alignment of Ab646-653 with parental Ab632 (SEQ ID NOs: 121-128).

FIGS. 9A-9H Treatment of 564 Igi mice with anti-CD21 monoclonal antibody ablates spontaneous autoreactive germinal centers. (FIG. 9A & 9B) 564 Igi mice were injected i.v. with PBS. (FIGS. 9C & 9D) 564 Igi mice were injected i.v. with 200 ug rat anti-CD21 (7G6 clone) for three days. (FIG. 9E) spleens from control and treated mice were stained for CD4+ T cells. (FIG. 9F) T follicular helper (Tfh) cells are identified with ICOS and CXCR5. (FIG. 9G) The proportion of germinal centers (GC) per follicle (Fo). (FIG. 9H) FACS analysis of Tfh cell frequency in the spleen. (n=2-4, graphs show mean plus SEM.) Results show diminishment of autoreactive GC and Tfh following treatment with anti-CD21.

FIG. 10 Sequence alignment of the CR2 (CCP1-2): C3d complex. Alignment of human, cynomolgus monkey, rat, mouse and bovine sequences with the human CR2-C3d complex structure (van den Elsen and Isenman, Science 2011). The residues involved in contacts between SCR1 and C3d are noted, as well as those involved in contacts between SCR2 and C3d (Reference 3OED). Residues in the interface (within 5 Angstrom radius) are highlighted in grey background (SEQ ID NOs: 129-134).

### DETAILED DESCRIPTION

Provided herein are methods and compositions comprising antibodies or antigen-binding fragments that bind to CD21 (e.g., at or near the C3 ligand binding site of CD21) and preferably inhibit CD21-mediated B cell activation and downstream cytokine release. Such antibodies or antigen-binding fragments can be used in the treatment of autoimmune diseases or disorders or other situations where suppression of B cell activation is desirable.

### Definitions

For convenience, certain terms employed in the specification, examples, and appended claims, are collected here. Unless defined otherwise, all technical and scientific terms used herein have the same meaning as commonly understood by one of ordinary skill in the art to which this invention belongs.

The term “antibody” refers to a protein that includes at least one immunoglobulin variable domain or immunoglobulin variable domain sequence. For example, an antibody can include a heavy (H) chain variable region (abbreviated herein as V_{H}), and a light (L) chain variable region (abbreviated herein as V_{L}). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)2, Fd fragments, Fv fragments, scFv, and domain antibodies (dAb) fragments (de Wildt et al., Eur J Immunol. 1996; 26 (3): 629-39.)) as well as complete antibodies. An antibody can have the structural features of IgA, IgG, IgE, IgD, IgM (as well as subtypes thereof). Antibodies can be from any source, but primate (human and non-human primate) and primatized are preferred

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, see also www.hgmp.mrc.ac.uk). Kabat definitions are used herein. Each VH and VL is typically composed of three CDRs and four FRs, arranged from amino-terminus to carboxy-terminus in the following order: FR1, CDR1, FR2, CDR2, FR3, CDR3, FR4.

As used herein, an “immunoglobulin variable domain sequence” refers to an amino acid sequence which can form the structure of an immunoglobulin variable domain such that one or more CDR regions are positioned in a conformation suitable for an antigen binding site. For example, the sequence can include all or part of the amino acid sequence of a naturally-occurring variable domain. For example, the sequence may omit one, two or more N- or C-terminal amino acids, internal amino acids, may include one or more insertions or additional terminal amino acids, 2 or may include other alterations. In one embodiment, a polypeptide that includes immunoglobulin variable domain sequence can associate with another immunoglobulin variable domain sequence to form an antigen binding site, e.g., a structure that preferentially interacts with a CD21 protein.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds or a linker. In IgGs, the heavy chain constant region includes three immunoglobulin domains, CH1, CH2 and CH3. The light chain constant region includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The light chains of the immunoglobulin can be of types kappa or lambda. In one embodiment, the antibody is glycosylated.

One or more regions of an antibody can be human or effectively human. For example, one or more of the variable regions can be human or effectively human. For example, one or more of the CDRs can be human, e.g., HC CDR1, HC CDR2, HC CDR3, LC CDR1, LC CDR2, and LC CDR3. Each of the light chain CDRs can be human. HC CDR3 can be human. One or more of the framework regions can be human, e.g., FR1, FR2, FR3, and FR4 of the HC or LC. For example, the Fc region can be human. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. In one embodiment, the framework (FR) residues of a selected Fab can be convertered to the amino-acid type of the corresponding residue in the most similar primate germline gene, especially the human germline gene. One or more of the constant regions can be human or effectively human. For example, at least 70, 75, 80, 85, 90, 92, 95, 98, or 100% of an immunoglobulin variable domain, the constant region, the constant domains (CH1, CH2, CH3, CL1), or the entire antibody can be human or effectively human.

All or part of an antibody can be encoded by an immunoglobulin gene or a segment thereof. Exemplary human immunoglobulin genes include the kappa, lambda, alpha (IgA1 and IgA2), gamma (IgG1, IgG2, IgG3, IgG4), delta, epsilon and mu constant region genes, as well as the many immunoglobulin variable region genes. Full-length immunoglobulin “light chains” (about 25 KDa or about 214 amino acids) are encoded by a variable region gene at the NH2-terminus (about 110 amino acids) and a kappa or lambda constant region gene at the COOH-terminus. Full-length immunoglobulin “heavy chains” (about 50 KDa or about 446 amino acids), are similarly encoded by a variable region gene (about 116 amino acids) and one of the other aforementioned constant region genes, e.g., gamma (encoding about 330 amino acids). The length of human HC varies considerably because HC CDR3 varies from about 3 amino-acid residues to over 35 amino-acid residues.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified to include a sufficient number of human framework amino acid positions such that the immunoglobulin variable region does not elicit an immunogenic response in a normal human (i.e., a subject that does not have an immune disorder). Descriptions of “humanized” immunoglobulins include, for example, U.S. Pat. No. 6,407,213 and U.S. Pat. No. 5,693,762.

As used herein, “binding affinity” refers to the apparent association constant or Ka. The Ka is the reciprocal of the dissociation constant (Kd). A binding protein can, for example, have a binding affinity of at least 10⁵, 10⁶, 10⁷, 10⁸, 10⁹, 10¹⁰ and 10¹¹ M⁻¹ for a particular target molecule, e.g., CD21. Higher affinity binding of a binding protein to a first target (e.g., CD21) relative to a second target (e.g., a non-specific protein) can be indicated by a higher Ka (or a smaller numerical value Kd) for binding the first target than the Ka (or numerical value Kd) for binding the second target. In such cases, the binding protein has specificity for the first target (e.g., a protein in a first conformation or mimic thereof) relative to the second target (e.g., the same protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, or 10⁵ fold.

In some embodiment, an antibody reagent (e.g., an antibody or antigen-binding domain thereof) specifically binds to CD21 present on the cell-surface with a K_{D} of 10⁻⁵ M (10000 nM) or less, e.g., 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less and binds to that target at least 100×, or 1000×, or 10,000× and preferably more strongly than it binds to an off-target or distinct cell-surface marker or protein. The person of ordinary skill in the art can determine appropriate conditions under which the polypeptide agents described herein selectively bind CD21 using any suitable methods, such as titration of the antibody reagent in a suitable cell binding assay. Binding affinity can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in TRIS-buffer (50 mM TRIS, 150 mM NaCl, 5 mM CaCl₂ at pH7.5). These techniques can be used to measure the concentration of bound and free binding protein as a function of binding protein (or target) concentration. The concentration of bound binding protein ([Bound]) is related to the concentration of free binding protein ([Free]) and the concentration of binding sites for the binding protein on the target where (N) is the number of binding sites per target molecule by the following equation:

$$[ Bound ] = N \cdot [ Free ] / ( ( 1 / Ka ) + [ Free ] ) .$$

It is not always necessary to make an exact determination of Ka, though, since sometimes it is sufficient to obtain a quantitative measurement of affinity, e.g., determined using a method such as ELISA or FACS analysis, is proportional to Ka, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

As used herein, an “antigen-binding fragment” refers that portion of an antibody that is necessary and sufficient for binding to a given antigen. At a minimum, an antigen binding fragment of a conventional antibody will comprise six complementarity determining regions (CDRs) derived from the heavy and light chain polypeptides of an antibody arranged on a scaffold that permits them to selectively binds the antigen. A commonly used antigen-binding fragment includes the V_{H} and V₂ domains of an antibody, which can be joined either via part of the constant domains of the heavy and light chains of an antibody, or, alternatively, by a linker, such as a peptide linker. Non-conventional antibodies, such as camelid and short antibodies have only 2 heavy chain sequences, denoted, for example V_{HH}. These can be used in a manner analogous to V_{H}/VL-containing antigen-binding fragments. Non-limiting examples of antibody fragments encompassed by the term antigen-binding fragment include: (i) a Fab fragment, having V_{L}, C_{L}, V_{H} and C_{H}1 domains; (ii) a Fab′ fragment, which is a Fab fragment having one or more cysteine residues at the C-terminus of the C_{H}1 domain; (iii) an Fd fragment having V_{H} and C_{H}1 domains; (iv) a Fd′ fragment having V_{H} and C_{H}1 domains and one or more cysteine residues at the C-terminus of the CH1 domain; (v) an Fv fragment having the V_{L} and V_{H} domains of a single arm of an antibody; (vi) a dAb fragment (Ward et al., Nature 341, 544-546 (1989)) which consists of a V_{H} domain; (vii) F(ab′)₂ fragments, a bivalent fragment including two Fab′ fragments linked by a disulphide bridge at the hinge region; (viii) single chain antibody molecules (e.g., single chain Fv; scFv) (Bird et al., Science 242:423-426 (1988); and Huston et al., PNAS (USA) 85:5879-5883 (1988)); (ix) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); and (x) “linear antibodies” comprising a pair of tandem Fd segments (V_{H}-C_{H}1-V_{H}-C_{H}1) which, together with complementary light chain polypeptides, form a pair of antigen binding regions (Zapata et al. Protein Eng. 8 (10): 1057-1062 (1995); and U.S. Pat. No. 5,641,870). The molecules of Fv, scFv or diabody can be stabilized by incorporating disulfide bridges linking the VH and VL domains. Minibodies comprising a scFv fragment linked to a CH3 domain can also be obtained. Other examples of binding fragments are Fab′, which differs from Fab fragments by the addition of some residues at the carboxyl terminus of the CHI domain of the heavy chain, including one or more cysteines of the hinge region of the antibody, and Fab′-SH, which is a Fab′ fragment in which the cysteine residue(s) of the constant domains carries a free thiol group.

In some embodiments of any one of the aspects, the antibody or the antigen binding fragment is an isolated antibody or antigen binding fragment. An “isolated antibody” is intended to refer to an antibody that is substantially free of other antibodies having different antigenic specificities (e.g., an isolated antibody that specifically binds to CD21 is substantially free of antibodies that specifically bind antigens other than CD21). An isolated antibody that specifically binds to CD21 can, however, have cross-reactivity to other antigens, such as to CD21 molecules from other species. Moreover, an isolated antibody can be substantially free of other cellular material and/or chemicals.

As used herein, the term “specificity” refers to the number of different types of antigens or antigenic determinants to which an antibody or antibody fragment thereof as described herein can bind. The specificity of an antibody or antibody fragment thereof can be determined based on affinity and/or avidity. The affinity, represented by the equilibrium constant for the dissociation (K_{D}) of an antigen with an antigen-binding protein, is a measure of the binding strength between an antigenic determinant and an antigen-binding site on the antigen-binding protein, such as an antibody or antibody fragment thereof: the lesser the value of the K_{D}, the stronger the binding strength between an antigenic determinant and the antigen-binding molecule. Alternatively, the affinity can also be expressed as the affinity constant (K_{A}), which is 1/K_{D}). Accordingly, an antibody or antibody fragment thereof as defined herein is said to be “specific for” a first target or antigen compared to a second target or antigen when it binds to the first antigen with an affinity (as described above, and suitably expressed, for example as a K_{D} value) that is at least 10 times, such as at least 100 times, and preferably at least 1000 times, and up to 10000 times or more better than the affinity with which said amino acid sequence or polypeptide binds to another target or polypeptide. Antibody affinities can be determined, for example, by a surface plasmon resonance based assay (such as the BIACORE assay described in PCT Application Publication No. WO2005/012359); enzyme-linked immunosorbent assay (ELISA); and competition assays (e.g., RIA's), for example.

As used herein, “avidity” is a measure of the strength of binding between an antigen-binding molecule (such as an antibody or antibody fragment thereof described herein) and the pertinent antigen. Avidity is related to both the affinity between an antigenic determinant and its antigen binding site on the antigen-binding molecule, and the number of pertinent binding sites present on the antigen-binding molecule. Typically, antigen-binding proteins (such as an antibody or portion of an antibody as described herein) will bind to their cognate or specific antigen with a dissociation constant (K_{D})) of 10⁻⁵ to 10⁻¹² moles/liter or less, such as 10⁻⁷ to 10⁻¹² moles/liter or less, or 10⁻⁸ to 10⁻¹² moles/liter (i.e., with an association constant (K_{A}) of 10⁵ to 10¹² liter/moles or more, such as 10⁷ to 10¹² liter/moles or 10⁸ to 10¹² liter/moles). Any K_{D} value greater than 10⁻⁴ mol/liter (or any K_{A} value lower than 10⁴ M⁻¹) is generally considered to indicate non-specific binding. The K_{D} for biological interactions which are considered meaningful (e.g., specific) are typically in the range of 10⁻¹⁰ M (0.1 nM) to 10⁻⁵ M (10000 nM). The stronger an interaction, the lower is its K_{D}. For example, a binding site on an antibody or portion thereof described herein will bind to the desired antigen with an affinity less than 500 nM, such as less than 200 nM, or less than 10 nM, such as less than 500 pM. Specific binding of an antigen-binding protein to an antigen or antigenic determinant can be determined in any suitable manner, including, for example, Scatchard analysis and/or competitive binding assays, such as radioimmunoassays (RIA), enzyme immunoassays (EIA) and sandwich competition assays, and the different variants thereof known in the art; as well as other techniques as mentioned herein.

Accordingly, as used herein, “selectively binds” or “specifically binds” refers to the ability of an antibody or antigen-binding fragment thereof as described herein to bind to a target, such as CD21, with a K_{D} 10⁻⁵ M (10000 nM) or less, e.g., 10⁻⁶ M, 10⁻⁷ M, 10⁻⁸ M, 10⁹ M, 10⁻¹⁰ M, 10⁻¹¹ M, 10⁻¹² M, or less. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay.

As used herein, the term “selectively inhibits” means that an agent, such as a bispecific antibody agent, inhibits, as that term is used herein, the association of a first ligand-receptor pair (e.g., CD21 and its cognate ligand) but does not substantially inhibit the association of a relevant second ligand-receptor pair.

The term “universal framework” refers to a single antibody framework sequence corresponding to the regions of an antibody conserved in sequence as defined by Kabat (“Sequences of Proteins of Immunological Interest”, US Department of Health and Human Services) or corresponding to the human germline immunoglobulin repertoire or structure as defined by Chothia and Lesk, J. Mol. Biol. 196:910-917 (1987). The Kabat database is now also maintained on the world wide web. The compositions and methods described herein provide for the use of a single framework, or a set of such frameworks, which have been found to permit the derivation of virtually any binding specificity though variation in the hypervariable regions alone. The universal framework can be a V_{L} framework (V_{λ} or V_{κ}), such as a framework that comprises the framework amino acid sequences encoded by the human germline DPK1, DPK2, DPK3, DPK4, DPK5, DPK6, DPK7, DPK8, DPK9, DPKIO, DPK12, DPK13, DPK15, DPK16, DPK18, DPK19, DPK20, DPK21, DPK22, DPK23, DPK24, DPK25, DPK26 or DPK 28 immunoglobulin gene segment. If desired, the V_{L} framework can further comprise the framework amino acid sequence encoded by the human germline J_{κ}1, J_{κ}2, J_{κ}3, J_{κ}4, or J_{κ}5 immunoglobulin gene segments. In other embodiments the universal framework can be a V_{H} framework, such as a framework that comprises the framework amino acid sequences encoded by the human germline DP4, DP7, DP8, DP9, DP10, DP31, DP33, DP38, DP45, DP46, DP47, DP49, DP50, DP51, DP53, DP54, DP65, DP66, DP67, DP68 or DP69 immunoglobulin gene segments. In some embodiments, the V_{H} framework can further comprise the framework amino acid sequence encoded by the human germline J_{H}1, J_{H}2, J_{H}3, J_{H}4, J_{H}4b, J_{H}5 or J_{H}6 immunoglobulin gene segments.

An “Fv” fragment is an antibody fragment which contains a complete antigen recognition and binding site. This region consists of a dimer of one heavy and one light chain variable domain in tight association, which can be covalent in nature, for example in a single-chain Fv or scFv (see below). It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

As used herein, “antibody variable domain” refers to the portions of the light and heavy chains of antibody molecules that include amino acid sequences of Complementarity Determining Regions (CDRs; i.e., CDR1, CDR2, and CDR3), and Framework Regions (FRs). V_{H} refers to the variable domain of the heavy chain. V_{L} refers to the variable domain of the light chain. For the methods and compositions described herein, the amino acid positions assigned to CDRs and FRs can be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

A “Fab” of “Fab fragment” fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

“Single-chain Fv” or “scFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains, which permits the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The expression “linear antibodies” refers to the antibodies described in Zapata et al., Protein Eng., 8(10): 1057-1062 (1995). Briefly, these antibodies comprise a pair of tandem Fd segments (VH-CHI-VH-CH1) which, together with complementary light chain polypeptides, form a pair of antigen binding regions. Linear antibodies can be bispecific or monospecific.

An “affinity matured” antibody is one with one or more alterations in one or more CDRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by V_{H} and V_{L} domain shuffling. Random mutagenesis of CDR and/or framework residues is described by: Barbas et al. Proc Nat. Acad. Sci, USA 91:3809-3813 (1994); Schier et al. Gene 169:147155 (1995); Yelton et al. J. Immunol. 155:1994-2004 (1995); Jackson et al., J. Immunol. 154(7):3310-9 (1995); and Hawkins et al., J. Mol. Biol. 226:889-896 (1992).

As used herein in relation to antibody domains, “complementary” refers to when two immunoglobulin domains belong to families of structures which form cognate pairs or groups or are derived from such families and retain this feature. For example, a V_{H} domain and a V_{L} domain of a natural antibody are complementary; two V_{H} domains are not complementary, and two V_{L} domains are not complementary. Complementary domains can be found in other members of the immunoglobulin superfamily, such as the V_{α} and V_{β} (or γ and δ) domains of the T cell receptor. Domains which are artificial, such as domains based on protein scaffolds which do not bind epitopes unless engineered to do so, are non-complementary. Likewise, two domains based on, for example, an immunoglobulin domain and a fibronectin domain are not complementary.

As used herein, the term “humanized antibody” refers to forms of antibodies that contain sequences from non-human (e.g., murine) antibodies as well as human antibodies. Such antibodies are chimeric antibodies which contain minimal sequence derived from non-human immunoglobulin. In general, the humanized antibody will ideally comprise substantially all of at least one, and typically two, variable domains, in which all or substantially all of the hypervariable loops correspond to those of a non-human immunoglobulin and all or substantially all of the framework (FR) regions are those of a human immunoglobulin sequence. The humanized antibody optionally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol 2:593-596 (1992)). The constant region, can if desired, include one or more modifications that modify or disrupt interaction of the human or humanized antibody with an Fc receptor, as described herein. Humanization can be essentially performed following the method of Winter and co-workers (Jones et al., Nature 321:522-525 (1986); Riechmann et al., Nature 332:323-3′27 (1988); Verhoeyen et al., Science 239:1534-1536 (1988)), by substituting rodent CDRs or CDR sequences for the corresponding sequences of a human antibody.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of autoimmune disease, or cancer. A subject can be male or female. In addition, a subject can be of any age, including a neonate, a toddler, a child, a pre-adolescent, an adolescent, or an adult or geriatric subject.

A subject can be one who has been previously diagnosed with or identified as suffering from or having a condition in need of treatment (e.g., an autoimmune disease such as lupus) or one or more complications related to such an autoimmune condition. Alternatively, a subject can also be one who has not been previously diagnosed as having an autoimmune disease or disorder or one or more complications related thereto. For example, a subject can be one who exhibits one or more risk such diseases or disorders.

A “subject in need” of treatment for a particular condition can be a subject having that condition, diagnosed as having that condition, or at risk of developing that condition.

The term “therapeutically effective amount” refers to an amount of an inhibitor as described herein, that is effective to inhibit CD21 activity, thereby treating an autoimmune condition, or reducing or relieving a symptom thereof. Amounts will vary depending on the specific disease or disorder, its state of progression, age, weight and gender of a subject, among other variables. Thus, it is not possible to specify an exact “effective amount”. However, for any given case, an appropriate “effective amount” can be determined by one of ordinary skill in the art using only routine experimentation.

As used herein, the terms “treat,” “treatment,” “treating,” or “amelioration” refer to therapeutic treatments, wherein the object is to reverse, alleviate, ameliorate, inhibit, slow down or stop the progression or severity of at least one symptom of an autoimmune disease or disorder (e.g., inflammation, fatigue, rash, fever, pain, weight loss etc.). The term “treating” includes reducing or alleviating at least one adverse effect or symptom of a disease or disorder. Treatment is generally “effective” if one or more symptoms or clinical markers of the autoimmune disease being treated is reduced. Alternatively, treatment is “effective” if the progression of the autoimmune disease or disorder is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total). The term “treatment” of a disease also includes providing relief from the symptoms or side-effects of the disease (including palliative treatment).

The term “prophylactically effective amount” refers to an amount of a CD21 antibody or antigen-binding fragment thereof which is effective, at dosages and for periods of time necessary, to achieve the desired prophylactic result, e.g., the amount of a CD21 antibody or fragment thereof to delay the onset of or reduce the intensity of at least one symptom of an autoimmune disease. Typically, since a prophylactic dose of a CD21 antibody is administered to a subject prior to onset of an autoimmune disease or at an early stage of an autoimmune disease, the dose necessary for a prophylactically effective amount can be less than the therapeutically effective amount. A prophylactically effective amount of CD21 antibody or fragment thereof is also one in which any toxic or detrimental effects of the compound are outweighed by the beneficial effects.

As used herein, the terms “prevent,” “preventing” and “prevention” refer to the avoidance or delay in manifestation of one or more symptoms or measurable markers of a disease or disorder, e.g., of an autoimmune disease. A delay in the manifestation of a symptom or marker is a delay relative to the time at which such symptom or marker manifests in a control or untreated subject with a similar likelihood or susceptibility of developing the disease or disorder. The terms “prevent,” “preventing” and “prevention” include not only the avoidance or prevention of a symptom or marker of the disease, but also a reduced severity or degree of any one of the symptoms or markers of the disease, relative to those symptoms or markers in a control or non-treated individual with a similar likelihood or susceptibility of developing the disease or disorder, or relative to symptoms or markers likely to arise based on historical or statistical measures of populations affected by the disease or disorder. By “reduced severity” is meant at least a 10% reduction in the severity or degree of a symptom or measurable disease marker, relative to a control or reference, e.g., at least 15%, 20%, 30%, 40%, 50%, 60%, 70%, 80%, 90%, 95%, 99% or even 100% (i.e., no symptoms or measurable markers).

The terms “decrease”, “reduce”, “reduction”, or “inhibit” are all used herein to mean a decrease by a statistically significant amount. In some embodiments, “reduce,” “reduction”, “decrease” or “inhibit” means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass complete inhibition or reduction as compared to a reference level. “Complete inhibition” is a 100% inhibition as compared to a reference level.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The “percent identity” or “percent homology” of two polynucleotide or two polypeptide sequences can be determined by comparing the sequences using the GAP computer program (a part of the GCG Wisconsin Package, version 10.3 (Accelrys, San Diego, Calif.)) using its default parameters.

The term “nucleic acid” refers to a deoxyribonucleotide or ribonucleotide and polymers thereof in either single strand or double strand form. The term “nucleic acid” is used interchangeably with gene, nucleotide, polynucleotide, cDNA, DNA, and mRNA. The polynucleotides can be in the form of RNA or DNA. Polynucleotides in the form of DNA, cDNA, genomic DNA, nucleic acid analogs, and synthetic DNA are within the scope of the present invention. Unless specifically limited the term encompasses nucleic acids containing known analogues of natural nucleotides that have similar binding propertied as the natural nucleic acid. Unless specifically limited, a particular nucleotide sequence also encompasses conservatively modified variants thereof (for example, those containing degenerate codon substitutions) and complementary sequences as well as the as well as the sequences specifically described.

The polynucleotides can be composed of any polyribonucleotide or polydeoxyribonucleotide, which can be unmodified RNA or DNA or modified RNA or DNA. For example, polynucleotides can be composed of single or double stranded regions, mixed single or double stranded regions. In addition, the polynucleotides can be triple stranded regions containing RNA or DNA or both RNA and DNA. Modified polynucleotides include modified bases, such as tritylated bases or unusual bases such as inosine. A variety of modification can be made to RNA and DNA, thus polynucleotide includes chemically, enzymatically, or metabolically modified forms.

The DNA may be double-stranded or single-stranded, and if single stranded, may be the coding (sense) strand or non-coding (anti-sense) strand. The coding sequence that encodes the polypeptide may be identical to the coding sequence provided herein or may be a different coding sequence, which sequence, as a result of the redundancy or degeneracy of the genetic code, encodes the same polypeptides as the DNA provided herein.

A variant DNA or amino acid sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

In some embodiments of the various aspects described herein, the degree of complementarity, when optimally aligned using a suitable alignment algorithm, is about or more than about 50%, 60%, 75%, 80%, 85%, 90%, 95%, 97.5%, 99%), or more. Optimal alignment can be determined with the use of any suitable algorithm for aligning sequences. Exemplary algorithms for determining optimal alignment include, but are not limited to, the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean ±1%.

### Autoimmune Diseases or Disorders

As used herein, an “autoimmune disease” refers to a class of diseases or disorders in which a subject's own antibodies react with host tissue or in which immune effector T cells are autoreactive to endogenous self-peptides and cause destruction of tissue. Thus an immune response is mounted against a subject's own antigens, referred to as self-antigens. A “self-antigen” as used herein refers to an antigen of a normal host tissue. Normal host tissue does not include cancer cells.

Accordingly, in some embodiments of these methods and all such methods described herein, the autoimmune diseases to be treated or prevented using the methods described herein, include, but are not limited to: rheumatoid arthritis, Crohn's disease or colitis, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenia purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjogren's syndrome, insulin resistance, and autoimmune diabetes mellitus (type 1 diabetes mellitus; insulin-dependent diabetes mellitus). Autoimmune disease has been recognized also to encompass atherosclerosis and Alzheimer's disease. In some embodiments of the aspects described herein, the autoimmune disease is selected from the group consisting of multiple sclerosis, type-I diabetes, gastritis, autoimmune hepatitis, autoimmune hemophilia, autoimmune lymphoproliferative syndrome (ALPS), autoimmune uveoretinitis, Guillain-Barre syndrome, psoriasis and myasthenia gravis.

In one embodiment, the autoimmune disease is systemic lupus erythematosus.

Systemic lupus erythematosus (SLE) is an autoimmune disease characterized by the production of antibodies to a number of nuclear antigens, including double-stranded DNA (dsDNA). In addition, anti-P2GPI antibodies can also be found in individuals with SLE. Autoantibodies that react with DNA are believed to play a role in the pathology of SLE and are closely associated with lupus nephritis. See, for example, Morimoto et al. (1982) J. Immunol. 139:1960-1965; Foster et al. (1993) Lab. Invest. 69:494-507; ter Borg et al. (1990) Arthritis Rheum. 33:634-643; and Bootsma et al. (1995) Lancet 345:1595-1599. Other clinical symptoms associated with SLE include malar rash, discoid rash, butterfly rash, photosensitivity, oral ulcers, arthritis, serositis (pleuritis and/or pericarditis), renal disorders (e.g., proteinuria), neurological disorders (e.g., seizures or psychosis), hematological disorders (e.g., hemolytic anemia, leukopenia, lymphopenia, thrombocytopenia), and immunological disorders (e.g., positive lupus erythematosus cell preparation, anti-DNA antibody to native DNA in abnormal titer, anti-SM nuclear antigen antibodies). Coutran et al. Pathologic Basis of Disease Fourth Ed. (1989).

Thryroiditis (or Hashimoto's thyroiditis) and Graves' disease are other autoimmune diseases which involve the thyroid and are thought to be caused by autoantibodies to thyroid-stimulating hormone (TSH) receptor. Pathological findings in the thyroid include excessive infiltration with chronic inflammatory cells, follicular rupture, eosinophilia, varying degrees of hyperplasia, and fibrosis. The clinical manifestations of chronic thyroiditis are variable but major syndromes are painless goiter, hypothyroidism, and a combination of both. Scientific American Medicine Chapter 6 Section VI pp. 6-8 and Chapter 3 Section I p. 16 (2001).

Myasthenia gravis is an autoimmune disease thought to be caused by autoantibodies to acetylcholine receptors (AchR). Autoantibodies to AchR can be detected and measured in serum of patients with myasthenia gravis. Clinical manifestations of myasthenia gravis include skeletal muscle weakness and fatigability. Muscle weakness can present as asymmetric ptosis and diplopia caused by the impaired ability to elevate the eyelids and movement of the extraocular muscles. Other physical symptoms include weak neck extensors, drooping of the head, facial snarl when patient attempts to smile due to weakness of facial and bulbar muscles, nasal or dysarthric and low-volume dysphonic speech, dysphagia which can result in choking or regurgitation, and skeletal muscle weakness which can cause difficulties in walking, climbing stairs, or carrying objects. The disease can be transmitted to experimental animals with patient's pathogenic IgG. Scientific American Medicine Chapter 6 Section VI pp. 6-8 and Chapter 11 Section III pp. 12-13 (2001).

Systemic scleroderma is a rare, slowly progressive rheumatic disease that is thought to be caused by autoantibodies to nuclear proteins such as SS-A (Ro), SS-B (La), Scl-70, and centromere. Systemic scleroderma can be diffuse or limited. The limited form of systemic scleroderma, or CREST (calcinosis, Raynaud's phenomenon, esophageal involvement, sclerodactyly, and telangiesctasias) can be fatal and involves internal organs less often than diffuse scleroderma. Clinical features of systemic scleroderma include swelling and thickening of the fingers and hand with possible involvement of the face, thickening of the skin, involvement of the trunk and arms proximal to the elbows. As systemic scleroderma progresses, clinical features include skin atrophy with possible loss of hair, sebaceous glands, and sweat glands; loss of pliability of the skin; hidebound skin where the skin is tightly drawn and bound to underlying structures; and limited mobility, especially in the fingers. Scientific American Medicine Chapter 6 Section VI pp. 6-8 and Chapter 15 Section V pp. 1-4 (2001).

Idiopathic thrombocytopenia purpura (ITP) is an autoimmune disorder which is characterized by rapid destruction of the platelets. It is thought that autoantibodies to proteins on platelets are formed and bind to the platelets that are subsequently removed by the reticuloendothelial system. The autoantibodies are frequently directed against the platelet glycoprotein (GP) IIb-IIIa receptor complex. Another target for autoantibodies in ITP is the GPIb receptor complex. Some clinical features of ITP include: presence of petechiae in the lower extremities, mild clinical bleeding consisting of purpura, epistaxis, gingival bleeding, menorrhagia, unpalpable spleen, and in case of several thrombocytopenia, blood blisters in the mouth. Scientific American Medicine Chapter 5 Section XIII pp. 2 (2001).

Polymyositis is a rheumatic disease which involves weakening of primarily skeletal muscle. Polymyositis is thought to be caused by autoantibodies to nuclear proteins such as Jo-1, histadyl-tRNA synthetase, threonyl-tRNA synthetase, PM-1, and Mi-2. Clinical features of polymyositis are weakening of proximal muscles and can also include possible pulmonary involvement such as aspiration pneumonia, interstitial lung disease; soft tissue calcification (seen most commonly in children); and association with another rheumatic disease such as Raynaud phenomenon. Scientific American Medicine Chapter 6 Section VI pp. 6-8 and Chapter 15 Section VI pp. 1-4 (2001).

### CD21

Human CD21, or complement receptor 2 (CR2), is a membrane glycoprotein of approximately 145-150 kDa which is expressed predominantly on mature B lymphocytes. Tedder, T. F., et al. J. Immunol. (1984) 133:678. Human CD21 is a receptor for complement fragments C3d, C3dg, and iC3b as well as for Epstein-Barr virus (EBV). Weis, J. J., et al. Proc. Natl. Acad. Sci. USA (1984) 81:881 and Fingeroth, J. D., et al. Proc. Natl. Acad. Sci. USA (1984) 81:4150. CD21 consists of approximately 15 to 16 extracellular short consensus repeats (SCR) of about 60 to 70 amino acids each, a transmembrane region of about 24 amino acids, and a short cytoplasmic portion of about 34 amino acids. In mice, CR2 and CR1 are produced by alternative splicing from the same gene, unlike in humans where CR2 and CR1 are unique products of different genes.

CD21 forms a noncovalent receptor complex with CD81 and CD19 that is important in B cell activation. On mature B cells, CD21 transmits costimulatory signals after cross-linking by polymeric C3d. Melchers, F., et al., Nature (1985) 317:264. Short consensus repeats 1 and 2 (SCR1 and/or SCR2) are thought to be a portion of CD21 which specifically recognizes C3d. It is believed that binding of complement fragments, for example C3d, to CD21 plays an important role in B cell responses by providing a link between the B cell antigen receptor and its co-receptor, thus making the B cell 100-to 10,000-fold more sensitive to the antigen. Janeway and Travers Immunobiology 3rd edition (1997) 8:43.

CD21 interacts with “C3d,” a complement fragment that is generated as part of the complement pathway of the immune system. C3 is abundant in the plasma. As part of the classical complement pathway, C3 is converted to C3a and C3b by C3 convertase. iC3b is a derivative of C3b and can be further converted to C3dg, for example, when bound to a pathogen as part of an opsonization. As part of the alternative complement pathway, C3b is produced at a significant rate from C3 by spontaneous cleavage. C3b can interact with factor I, a serine protease that circulates in active form and cleaves C3b first into iC3b and then further to C3dg. C3dg can be further degraded to yield C3d. Both C3d and C3dg are capable of binding to CD21. It is understood that C3d and C3dg can be used interchangeably herein. Reference to C3d is understood to also include C3dg and iC3b. iC3b includes the amino acid sequence of C3dg and C3d and binds to CD21 with similar affinity. iC3b is cleaved by proteases to yield C3dg. C3dg has several additional amino acids at the carboxy terminal end which are cleaved by cellular proteases to yield C3d. The structure of C3d is well known in the art (see, for example, Nagar, B. et al. Science 1998. 280, 1277-1281). C3d can be made recombinantly (for example, using published sequences from sources such as Genbank) or obtained in purified form from commercial sources (Calbiochem-Novabiochem; San Diego, Calif., Catalog #204870).

An agent that “reduces or inhibits CD21 activity” or “reduces or inhibits CD21/C3d mediated B cell activation” is an antibody or antigen-binding fragment thereof as described herein that reduces interaction of CD21 with one or more of its cognate receptors or ligands (e.g., CD3d). For example, an anti-CD21 antibody or antigen-binding fragment as described herein reduces the extent of CD21/C3d mediated B cell activation as compared to the extent of CD21/C3d mediated B cell activation in the presence of C3d but without the anti-CD21 binding agent. The inhibition of B cell activation can be a partial reduction in activity of B cells such as reduction in the production of antibodies. The inhibition or suppression of CD21/C3d mediated B cell activation can be partial or total. Methods of indicating CD21/C3d mediated B cell activation are known in the art and/or are described herein. It is understood that “B cell activation” includes the activation of resting B cells, activation of non-antibody-secreting B cells, and sustained and/or enhancement of activation state of B cells which are already activated (e.g., plasma cells).

In one embodiment, the anti-CD21 antibody or antigen-binding fragment thereof as described herein can “interfere with C3d binding to CD21” or “reduce binding of C3d to CD21” (e.g., reduces the extent of interaction between C3d ligand and CD21 as compared with otherwise same conditions without the agent). Methods for determining C3d binding to CD21 are disclosed herein. An agent which “interferes with C3d binding to CD21” can reduce the levels of antibodies such as anti-dsDNA antibodies when appropriately administered.

### CD21 and Systemic Lupus Erythematosus

A major goal in SLE research is to identify a therapy that “shuts-off” or ablates the spontaneous B cell response to self-antigen, particularly those responses that occur in germinal centers (GC), preventing the formation of affinity-matured, pathogenic autoantibody. Within GC, activated B cells differentiate into high affinity isotype-switched plasma cells and form long-term memory B cells (Allen et al., 2007; Hauser et al., 2007a; Hauser et al., 2007b; MacLennan, 1994; Schwickert et al., 2007), events that correlate with poor prognostic outcomes when GC reactions are directed against auto-antigens. In humoral responses to infection, follicular dendritic cells (FDC) are important in retention of antigen and provide the necessary environment for GC development. FDC retain foreign antigen for extensive periods via complement receptor CD21. To limit expansion of auto-reactive B cell clones arising during somatic hypermutation (SHM), newly-formed GC B cells require contact with cognate antigen and the complement ligand C3d for their persistence (Fischer et al., 1998; Goodnow et al., 1995). Thus, blockade at this critical checkpoint provides a therapeutic target to prevent the development of auto-reactive effector B cells. Without wishing to be bound by theory, interrupting contact between the B cell co-receptor CD21 and its C3d ligand prevents the survival of GC B cells and dissolves autoimmune GC reactions. For example, in lupus patients where auto-reactive B cells are undergoing spontaneous expansion within their secondary lymphoid tissues, transient blockade of B cell co-receptor with a monoclonal antibody specific for the C3d binding site of CD21 leads to rapid elimination of the auto-reactive B cells and limits their further differentiation into mature, high affinity antibody producing cells. Thus, treatment with anti-CD21 therapy has two important effects: (i) it can block B cell co-receptor contact with C3d ligand; and (ii) strips auto-antigen immune complexes from FDC disrupting signaling of GC B cells. Significantly, blockade of the CD21 receptor has the potential to “shut-off” the source of pathogenic antibodies without eliminating the non-self-reactive B cell repertoire.

A major concern with blockade of the immune system is that it increases the susceptibility of the individual to infection. This is a particular concern with lupus patients where secondary infections with bacteria such as Streptococcus pneumoniae can be fatal. Therefore, a balance between a therapy that is sufficiently suppressive to reduce autoimmunity but one that permits a host response to infection is most desirable. One potential advantage of the anti-CD21 therapy is that it blocks access of self-reactive GC B cells to self-antigen and limits their differentiation into pathogenic effector cells, but does not alter early primary response to infection (Gonzalez et al., 2008). B cell depletion therapy by Rituximab in patients with SLE results in reductions in anti dsDNA and anti-nucleosome antibodies but not in anti-pathogen antibodies, indicating that B cells generating these autoantibodies experience a more rapid turnover and may be more dependent on ongoing GC reaction (Cambridge et al. 2006). In addition, autoantibody plasma cells may be not as long-lived in the bone marrow as non-self plasma cells. These differences confer to a GC-targeted therapy a more desirable therapeutic profile than that of other B-cell directed therapies. Therefore, relative to more global therapies such as steroids, anti-CD20 (Rituximab) or anti-BAFF (Belimumab) treatment, anti-CD21 therapy can achieve greater efficacy and be less suppressive to the immune system.

### Antibodies

Provided herein are exemplary antibodies and antigen-binding fragments that bind to CD21. In some embodiments, such antibodies or antigen-binding fragments can inhibit or reduce CD21 activity or interaction of CD21 with its cognate ligand (e.g., C3d). For example, the antibody or antigen-binding fragment binds to or sterically blocks the C3 ligand binding site on CD21.

The antibody or antigen-binding fragment described herein can be in form of a full antibody or a fragment thereof, e.g., a Fab, F(ab′)2, Fv, or a single chain Fv fragment (scFv). For example, the antibody or antigen-binding fragment can be selected from the group consisting of Fv, Fab, Fab′, F(ab)2, F(ab′)2, single-chain antibody, single-chain antibody (scFV), sc(Fv)2, monovalent antibody lacking hinge region, whole antibody, disulfide-stabilized Fv (dsFv), diabody dAb, a bivalent or bispecific antibody, a nanobody, a monoclonal antibody, a humanized antibody, a human antibody, a recombinant antibody, or a chimeric antibody.

In some embodiments of any one of the aspects, the antibody or antigen-binding fragment thereof comprises a heavy chain variable region (V_{H}) comprising a complementarity determining region 1 (CDR_H1) sequence that differs by no more than four, preferably no more than three, more preferably no more than two, most preferably no more than one amino acid from the amino acid sequence G-G-X₁-X₂-X₃-X₄-X₅-A-I-S (SEQ ID NO: 102), wherein X₁ is T or E; X₂is For L; X₃is S, or Q; X₄ is T, D, or S; and X₅ is Y or L. For example, the antibody or antigen-binding fragment thereof comprises a heavy chain variable region comprising a CDR_H1 sequence having an amino acid sequence that differs by no more than four, preferably no more than three, more preferably no more than two, most preferably no more than one amino acid from an amino acid sequence selected from the group consisting of: SEQ ID NOs: 2, 30, 36, 44, 81, 84, 87, 90, 93, 96, and 99.

In some embodiments of any one of the aspects, the antibody or antigen-binding fragment thereof comprises a heavy chain variable region comprising a complementarity determining region 2 (CDR_H2) sequence that differs by no more than four, preferably no more than three, more preferably no more than two, most preferably no more than one amino acid from the amino acid sequence RIIPILGIAN YAQKFQG (SEQ ID NO: 3).

In some embodiments of any one of the aspects, the antibody or antigen-binding fragment thereof comprises a heavy chain variable region comprising a complementarity determining region 3 (CDR_H3) sequence that differs by no more than four, preferably no more than three, more preferably no more than two, most preferably no more than one amino acid from the amino acid sequence E-X₆-D-X₇-S-G-Y-Y-Q-D (SEQ ID NO: 103), wherein X₆ is D or E; and X₇ is S or I. For example, the antibody or antigen-binding fragment thereof comprises a heavy chain variable region comprising a CDR_H3 sequence having an amino acid sequence that differs by no more than four, preferably no more than three, more preferably no more than two, most preferably no more than one amino acid from an amino acid sequence selected from the group consisting of: SEQ ID NOs: 4, 37, and 51.

In some embodiments of any one of the aspects, the antibody or antigen-binding fragment thereof comprises a light chain variable region (V_{L}) comprising a complementarity determining region 1 (CDR_L1) sequence that differs by no more than four, preferably no more than three, more preferably no more than two, most preferably no more than one amino acid from the amino acid sequence R-A-G-Q-X₈-I-X₉-X₁₀-Y-L-A (SEQ ID NO: 104), wherein X₈ is S or K; X₉ is N or S; and X₁₀ is S, H, or R. For example, the antibody or antigen-binding fragment thereof further comprises a light chain variable region comprising a CDR_L1 sequence that differs by no more than four, preferably no more than three, more preferably no more than two, most preferably no more than one amino acid of an amino acid sequence selected from the group consisting of: SEQ ID NOs: 15, 33, 54, 59, 62, and 65.

In some embodiments of any one of the aspects, the antibody or antigen-binding fragment thereof comprises a light chain variable region comprising a complementarity determining region 2 (CDR_L2) sequence that differs by no more than four, preferably no more than three, more preferably no more than two, most preferably no more than one amino acid from the amino acid sequence D-A-S-X₁₁-R-A-T (SEQ ID NO: 105), wherein X₁₁ is S or T. For example, the antibody or antigen-binding fragment thereof further comprises a light chain variable region comprising a CDR_L2 sequence that differs by no more than four, preferably no more than three, more preferably no more than two, most preferably no more than one amino acid of an amino acid sequence selected from the group consisting of: SEQ ID NOs: 16, and 68.

In some embodiments of any one of the aspects, the antibody or antigen-binding fragment thereof comprises a light chain variable region comprising a complementarity determining region 3 (CDR_L3) sequence that differs by no more than four, preferably no more than three, more preferably no more than two, most preferably no more than one amino acid from the amino acid sequence Q-Q-Y-E-Y-W-Y-X₁₂ (SEQ ID NO: 106), wherein X₁₂ is S or T. For example, the antibody or antigen-binding fragment thereof further comprises a light chain variable region comprising a CDR_L3 sequence that differs by no more than four, preferably no more than three, more preferably no more than two, most preferably no more than one amino acid of an amino acid sequence selected from the group consisting of SEQ ID NOs: 17, and 55.

In addition to the CDRs, the heavy chain variable region can also comprise one or more framework (FR) sequences. Accordingly, in some embodiments of any one of the aspects, the heavy chain variable region further comprises a framework region 1 (FR1, also referred to as FW_H1 herein) sequence that differs by no more than four, preferably no more than three, more preferably no more than two, most preferably no more than one amino acid from SEQ ID NO: 10.

In some embodiments of any one of the aspects, the heavy chain variable region further comprises a FR2 (also referred to as FW_H2 herein) sequence that differs by no more than four, preferably no more than three, more preferably no more than two, most preferably no more than one amino acid from SEQ ID NO: 11.

In some embodiments of any one of the aspects, the heavy chain variable region further comprises a FR3 (also referred to as FW_H3 herein) sequence that differs by no more than four, preferably no more than three, more preferably no more than two, most preferably no more than one amino acid from SEQ ID NO: 12.

In some embodiments of any one of the aspects, the heavy chain variable region further comprises a FR4 (also referred to as FW_H4 herein) sequence that differs by no more than four, preferably no more than three, more preferably no more than two, most preferably no more than one amino acid from SEQ ID NO: 13.

In some embodiments of any one of the aspects, the antibody or antigen-binding fragment thereof comprises a heavy chain variable region comprising an amino acid sequence having at least 85%, 86%, 87%, 88%, 89%, 90%, 91%, 92%, 93%, 94%, 95%, 96%, 97%, 98% or 99% identity to an amino acid sequence selected from the group consisting of SEQ ID NOs: 9, 31, 38, 45, 47, 49, 52, 77, 82, 85, 88, 91, 94, 97 and 100. For example, the V_{H} region comprises an amino acid sequence having at least 90%, 91%, 92%, 93%, 94%, 95%, 96%, 97%, 98% or 99% identity to an amino acid sequence selected from the group consisting of SEQ ID NOs: 9, 31, 38, 45, 47, 49, 52, 77, 82, 85, 88, 91, 94, 97 and 100. In some embodiments, the V_{H} region comprises an amino acid sequence having at least 95%, 96%, 97%, 98% or 99% identity to an amino acid sequence selected from the group consisting of SEQ ID NOs: 9, 31, 38, 45, 47, 49, 52, 77, 82, 85, 88, 91, 94, 97 and 100.

The antibody or antigen-binding fragment thereof described herein can comprise antibody constant regions or parts thereof. For example, the heavy chain variable region can be attached at its C-terminal to all or part of a heavy chain like IgA, IgD, IgE, IgG, and IgM, and any isotype subclass. Thus, the antibody or antigen-binding fragment thereof described herein can include the CH1, hinge, CH2, CH3, and/or CH4 regions of the heavy chain.

In some embodiments of any one of the aspects, the antibody or antigen-binding fragment thereof comprises a heavy chain constant region comprising a CHI region comprising an amino acid sequence having at least 85%, 86%, 87%, 88%, 89%, 90%, 91%, 92%, 93%, 94%, 95%, 96%, 97%, 98% or 99% identity to SEQ ID NO: 5. For example, the CHI region comprises an amino acid sequence having at least 90%, 91%, 92%, 93%, 94%, 95%, 96%, 97%, 98% or 99% identity to SEQ ID NO: 5. In some embodiments, the CHI region comprises an amino acid sequence having at least 95%, 96%, 97%, 98% or 99% identity to SEQ ID NO: 5.

In some embodiments of any one of the aspects, the antibody or antigen-binding fragment thereof comprises a heavy chain constant region comprising a CH2 region comprising an amino acid sequence having at least 85%, 86%, 87%, 88%, 89%, 90%, 91%, 92%, 93%, 94%, 95%, 96%, 97%, 98% or 99% identity to SEQ ID NO: 7. For example, the CH2 region comprises an amino acid sequence having at least 90%, 91%, 92%, 93%, 94%, 95%, 96%, 97%, 98% or 99% identity to SEQ ID NO: 7. In some embodiments, the CH2 region comprises an amino acid sequence having at least 95%, 96%, 97%, 98% or 99% identity to SEQ ID NO: 7.

In some embodiments of any one of the aspects, the antibody or antigen-binding fragment thereof comprises a heavy chain constant region comprising a CH3 region comprising an amino acid sequence having at least 85%, 86%, 87%, 88%, 89%, 90%, 91%, 92%, 93%, 94%, 95%, 96%, 97%, 98% or 99% identity to SEQ ID NO: 8 or 40. For example, the CH3 region comprises an amino acid sequence having at least 90%, 91%, 92%, 93%, 94%, 95%, 96%, 97%, 98% or 99% identity to SEQ ID NO: 8 or 40. In some embodiments, the CH3 region comprises an amino acid sequence having at least 95%, 96%, 97%, 98% or 99% identity to SEQ ID NO: 8 or 40.

In some embodiments of any one of the aspects, the antibody or antigen-binding fragment thereof comprises a heavy chain constant region comprising a hinge region (CH1_CH2_hinge) comprising an amino acid sequence having at least 85%, 86%, 87%, 88%, 89%, 90%, 91%, 92%, 93%, 94%, 95%, 96%, 97%, 98% or 99% identity to SEQ ID NO: 6. For example, the hinge region comprises an amino acid sequence having at least 90%, 91%, 92%, 93%, 94%, 95%, 96%, 97%, 98% or 99% identity to SEQ ID NO: 6. In some embodiments, the hinge region comprises an amino acid sequence having at least 95%, 96%, 97%, 98% or 99% identity to SEQ ID NO: 6.

In some embodiments, of any one of the aspects, the antibody or antigen-binding fragment thereof comprises a heavy chain amino acid sequence having at least 85%, 86%, 87%, 88%, 89%, 90%, 91%, 92%, 93%, 94%, 95%, 96%, 97%, 98% or 99% identity to an amino acid sequence selected from the group consisting of SEQ ID NOs: 1, 29, 35, 39, 41, 42, 43, 46, 48, 50, 76, 80, 83, 86, 89, 92, 95, 98 and 101. For example, the antibody or antigen-binding fragment thereof comprises a heavy chain amino acid sequence having at least 90%, 91%, 92%, 93%, 94%, 95%, 96%, 97%, 98% or 99% identity to an amino acid sequence selected from the group consisting of SEQ ID NOs: 1, 29, 35, 39, 41, 42, 43, 46, 48, 50, 76, 80, 83, 86, 89, 92, 95, 98 and 101. In some embodiments, the antibody or antigen-binding fragment thereof comprises a heavy chain amino acid sequence having at least 95%, 96%, 97%, 98% or 99% identity to an amino acid sequence selected from the group consisting of SEQ ID NOs: 1, 29, 35, 39, 41, 42, 43, 46, 48, 50, 76, 80, 83, 86, 89, 92, 95, 98 and 101.

Like the heavy chain variable region, the light chain variable region can also comprise one or more framework (FR) sequences. Accordingly, in some embodiments of any one of the aspects, the light chain variable region further comprises a FR1 (also referred to as FW_L1 herein) sequence that differs by no more than four, preferably no more than three, more preferably no more than two, most preferably no more than one amino acid from SEQ ID NO: 20, 26 or 57.

In some embodiments of any one of the aspects, the light chain variable region further comprises a FR2 (also referred to as FW_L2 herein) sequence that differs by no more than four, preferably no more than three, more preferably no more than two, most preferably no more than one amino acid from SEQ ID NO: 21 or 27.

In some embodiments of any one of the aspects, the light chain variable region further comprises a FR3 (also referred to as FW_L3 herein) sequence that differs by no more than four, preferably no more than three, more preferably no more than two, most preferably no more than one amino acid from SEQ ID NO: 23 or 29.

In some embodiments of any one of the aspects, the light chain variable region further comprises a FR4 (also referred to as FW_L4 herein) sequence that differs by no more than four, preferably no more than three, more preferably no more than two, most preferably no more than one amino acid from SEQ ID NO: 23.

In some embodiments of any one of the aspects, the antibody or antigen-binding fragment thereof comprises a light chain variable region comprising an amino acid sequence having at least 85%, 86%, 87%, 88%, 89%, 90%, 91%, 92%, 93%, 94%, 95%, 96%, 97%, 98% or 99% identity to an amino acid sequence selected from the group consisting of SEQ ID NOs: 19, 25, 34, 56, 60, 63, 66, 69, 71, 73, 75 and 79. For example, the V_{L} region comprises an amino acid sequence having at least 90%, 91%, 92%, 93%, 94%, 95%, 96%, 97%, 98% or 99% identity to an amino acid sequence selected from the group consisting of SEQ ID NOs: 19, 25, 34, 56, 60, 63, 66, 69, 71, 73, 75 and 79. In some embodiments of any one of the aspects, the V_{L} region comprises an amino acid sequence having at least 95%, 96%, 97%, 98% or 99% identity to an amino acid sequence selected from the group consisting of SEQ ID NOs: 19, 25, 34, 56, 60, 63, 66, 69, 71, 73, 75 and 79.

The antibody or antigen-binding fragment thereof described herein can comprise antibody constant regions of parts thereof. For example, the light chain variable region (V_{L}) can be attached at its C-terminal end to constant antibody light chain domains (C_{L}). Accordingly, in some embodiments of any one of the aspects, the antibody or antigen-binding fragment thereof comprises a light chain constant region (C_{L}) comprising an amino acid sequence having at least 85%, 86%, 87%, 88%, 89%, 90%, 91%, 92%, 93%, 94%, 95%, 96%, 97%, 98% or 99% identity to SEQ ID NO: 18. For example, the C_{L} region comprises an amino acid sequence having at least 90%, 91%, 92%, 93%, 94%, 95%, 96%, 97%, 98% or 99% identity to SEQ ID NO: 18. In some embodiments, the C_{L} region comprises an amino acid sequence having at least 95%, 96%, 97%, 98% or 99% identity to SEQ ID NO: 18.

In some embodiments, of any one of the aspects, the antibody or antigen-binding fragment thereof comprises a light chain amino acid sequence having at least 85%, 86%, 87%, 88%, 89%, 90%, 91%, 92%, 93%, 94%, 95%, 96%, 97%, 98% or 99% identity to an amino acid sequence selected from the group consisting of SEQ ID NOs: 24, 32, 53, 58, 61, 64, 67, 70, 72, 74 and 78. For example, the antibody or antigen-binding fragment thereof comprises a light chain amino acid sequence having at least 90%, 91%, 92%, 93%, 94%, 95%, 96%, 97%, 98% or 99% identity to an amino acid sequence selected from the group consisting of SEQ ID NOs: 24, 32, 53, 58, 61, 64, 67, 70, 72, 74 and 78. In some embodiments, the antibody or antigen-binding fragment thereof comprises a light chain amino acid sequence having at least 95%, 96%, 97%, 98% or 99% identity to an amino acid sequence selected from the group consisting of SEQ ID NOs: 24, 32, 53, 58, 61, 64, 67, 70, 72, 74 and 78.

In another embodiment of this aspect and all other aspects provided herein, the antibody or antigen binding fragment reduces CD21 activity and/or B cell activation by at least 20% (e.g., at least 30%, at least 40%, at least 50%, at least 60%, at least 70%, at least 80%, at least 90%, at least 95%, at least 98%, at least 99% or even 100% (i.e., complete inhibition or CD21 activity below detectable limits) as compared to CD21 activity and/or B cell activation in the absence of the antibody or antigen-binding fragment. In some embodiments, the antibody or antigen-binding fragment interferes with B lymphocyte co-receptor signaling; removes immune complexes from follicular dendritic cell (FDC), thereby removing availability of antigen and reducing cytokine secretion.

In one embodiment, an inhibitory antibody or antigen binding fragment thereof binds the CD21 (e.g., the CD21 extracellular domain) and/or inhibits or otherwise interferes with binding of natural ligands to the CD21 receptor molecule. Other mechanisms, such as interference with receptor interaction with other (co)regulatory molecules can also be effective; the key is that binding of the antibody reagent inhibits receptor signaling, and this can be verified in an appropriate cell culture assay.

A variety of suitable antibody reagent formats are known in the art, such as complete antibodies, e.g., an IgG, or modified forms or fragments of such antibodies, including, as non-limiting examples, single chain antibodies, heterodimers of antibody heavy chains and/or light chains, an Fv fragment (e.g., single chain Fv (scFv), a disulfide bonded Fv), a Fab fragment, a Fab′ fragment, a F(ab′)2 fragment), a single variable domain (e.g., VH, VL, VHH), a dAb, and modified versions of any of the foregoing (e.g., modified by the covalent attachment of polyalkylene glycol (e.g., polyethylene glycol, polypropylene glycol, polybutylene glycol) or other suitable polymer). Antibody reagents or constructs can, if desired, be linked to an antibody Fc region, comprising one or both of CH2 and CH3 domains, and optionally, a hinge region. Such linkage can provide benefits such as increased serum half-life or promotion of effector function(s). Alternatively, antibody reagents or constructs can be fused to a carrier such as serum albumin to promote increased serum half-life.

Antibodies suitable for practicing the methods described herein are preferably monoclonal, and can include, but are not limited to, human, humanized or chimeric antibodies, including single chain antibodies, Fab fragments, F(ab′) fragments, fragments produced by a Fab expression library, and/or binding fragments of any of the above. Antibody reagents also include immunoglobulin molecules and immunologically active portions of immunoglobulin molecules, i.e., molecules that contain at least one, at least two, at least three or more antigen binding sites that specifically bind TIM-3 and one or more myeloid cell markers. Such immunoglobulin molecules can be of any type (e.g., IgG, IgE, IgM, IgD, IgA and IgY), class (e.g., IgG1, IgG2, IgG3, IgG4, IgA1 and IgA2) or subclass of immunoglobulin molecule, as understood by one of skill in the art.

In one embodiment, the antibody that binds CD21 is a monoclonal antibody.

Additional types of antibodies include, but are not limited to, chimeric, humanized, and human antibodies. For application in man, it is often desirable to reduce immunogenicity of antibodies originally derived from other species, like mouse. This can be done by construction of chimeric antibodies, or by a process called “humanization”. In this context, a “chimeric antibody” is understood to be an antibody comprising a domain (e.g. a variable domain) derived from one species (e.g. mouse) fused to a domain (e.g. the constant domains) derived from a different species (e.g. human).

The term “monoclonal antibody” as used herein refers to a population of antibodies that comprise an identical antigen-binding domain. In some embodiments, a monoclonal antibody can be produced by a single B cell clone, B cell hybrodima or its equivalent. Such a cell produces only one antibody, such that all antibodies produced by such a clone have the same antigen-binding domain. In contrast to polyclonal antibody preparations that typically include different antibodies directed against different determinants (epitopes) on a given target antigen, each monoclonal antibody is directed against a single determinant on the antigen. The term “monoclonal antibody” as used herein is not limited to antibodies produced through hybridoma technology. It is to be understood that the term “monoclonal antibody” refers to an antibody that is derived from a single clone, including any eukaryotic, prokaryotic or phage clone, and not the method by which the antibody is produced. For example, the monoclonal antibodies to be used in accordance with the methods and compositions described herein can be made by the hybridoma method first described by Kohler et al., Nature 256:495 (1975), or can be made by recombinant DNA methods (see, e.g., U.S. Pat. No. 4,816,567). The “monoclonal antibodies” can also be isolated from phage antibody libraries using the techniques described in Clackson et al., Nature 352:624-628 (1991) or Marks et al., J. Mol. Biol. 222:581-597 (1991), for example. A wide variety of methods for producing constructs with the antigen-binding domain of monoclonal antibodies are known to those of ordinary skill in the art.

Antibodies useful in the present methods can be described or specified in terms of the particular CDRs they comprise (see e.g., Tables 1 and 2). The compositions and methods described herein encompass the use of an antibody or derivative thereof comprising a heavy or light chain variable domain, where the variable domain comprises (a) a set of three CDRs, and (b) a set of four framework regions, and in which the antibody or antibody derivative thereof specifically binds CD21.

Also provided herein are chimeric antibody derivatives of an anti-CD21 polypeptide agent, i.e., antibody molecules in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). Chimeric antibody molecules can include, for example, one or more antigen binding domains from an antibody of a mouse, rat, or other species, with human constant regions. A variety of approaches for making chimeric antibodies have been described and can be used to make chimeric antibodies containing the immunoglobulin variable region which recognizes the selected antigens, on the surface of differentiated cells or tumor-specific cells. See, for example, Takeda et al., 1985, Nature 314:452; Cabilly et al., U.S. Pat. No. 4,816,567; Boss et al.; Tanaguchi et al., European Patent Publication EP171496; European Patent Publication 0173494, United Kingdom patent GB 2177096B).

The CD21 antibodies described herein can also include humanized antibody derivatives. Humanized forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

In some embodiments, antibodies described herein include derivatives that are modified, i.e., by the covalent attachment of another type of molecule to the antibody that does not prevent the antibody from binding to its target. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of turicamycin, etc. Additionally, the derivative can contain one or more non-classical amino acids.

Antibodies or antigen-binding fragments thereof that bind CD21 as described herein can be generated by any suitable method known in the art. Antibodies can be produced in bacteria, yeast, fungi, protozoa, insect cells, plants, or mammalian cells (see e.g., Frenzel et al. (2013) Front Immunol. 4:217). A mammalian expression system is generally preferred for manufacturing most of therapeutic proteins, such as antibodies, as they require post-translational modifications. A variety of mammalian cell expression systems are now available for expression of antibodies, including but not limited to immortalized Chinese hamster ovary (CHO) cells, mouse myeloma (NSO), mouse L-cells, myeloma cell lines like J558L and Sp2/0, baby hamster kidney (BHK), or human embryo kidney (HEK-293).

In certain embodiments, the CD21 antibodies described herein are produced in antibody producing cells isolated and/or derived from a human or mammalian subject, which permits manipulation and serves as the basis for production of mammalian, including human, hybridoma cell lines. Typically, the host animal is inoculated intraperitoneally with an amount of immunogen, e.g., Raji cells or SCR1/SCR2 fusion protein, sufficient to generate an immunogenic response and then boosted with similar amounts of the immunogen. Lymphoid cells, preferably spleen lymphoid cells from the host, are collected a few days after the final boost and a cell suspension is prepared therefrom for use in the fusion.

In some embodiments, the CD21 antibody or antigen-binding fragment thereof is a humanized antibody. Methods for generating humanized antibodies are known to those of skill in the art and are not described in detail herein. The following four general steps for generating a humanized antibody include: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; and 6,331,415.

In yet another alternative, fully human antibodies can be obtained by using commercially available mice that have been engineered to express specific human immunoglobulin proteins. Transgenic animals that are designed to produce a more desirable (e.g., fully human antibodies) or more robust immune response can also be used for generation of humanized or human antibodies. Examples of such technology are Xenomouse™ from Abgenix, Inc. (Fremont, Calif.) and HuMAb-Mouse® and TC Mouse™ from Medarex, Inc. (Princeton, N.J.).

In an alternative, antibodies can be made recombinantly and expressed using any method known in the art. Antibodies can be made recombinantly by expressing the gene sequence of a given anti-CD21 antibody recombinantly in host cells (e.g., CHO cells). Another method which can be employed is to express the antibody sequence in plants (e.g., tobacco) or transgenic milk. Methods for expressing antibodies recombinantly in plants or milk have been disclosed. See, for example, Peeters, et al. (2001) Vaccine 19:2756; Lonberg, N. and D. Huszar (1995) Int.Rev.Immunol 13:65; and Pollock, et al. (1999) J Immunol Methods 231:147. Methods for making derivatives of antibodies, e.g., humanized, single chain, etc. are known in the art. In another alternative, antibodies can be made recombinantly by phage display technology. See, for example, U.S. Pat. Nos. 5,565,332; 5,580,717; 5,733,743; 6,265,150; and Winter et al., Annu. Rev. Immunol. (1994) 12:433-455. In addition, binding of CD21 binding proteins to cells expressing CD21 can be characterized in a number assays known in the art, including FACS (Fluorescence Activated Cell Sorting), immunofluorescence, and immunocytochemistry. CD21 binding protein is contacted with cells which express or contain CD21 (e.g., B cells), and binding is detected in accordance with the method being used.

In certain embodiments, the anti-CD21 antibodies (or antigen binding fragment thereof) described herein exhibits 1, 2, 3, or 4 of the following characteristics: (a) binds to CD21; (b) binds to one or more epitopes of CD21 to which C3d binds; (c) binds to CD21 to inhibit CD21/C3d-mediated B cell activation (d) binds to CD21 to inhibit CD21/C3d-mediated B cell activation and lower levels of antibody production.

Immunoassays and flow cytometry sorting techniques such as fluorescence activated cell sorting (FACS) can also be employed to isolate antibodies that are specific for CD21 and that bind the SCR1 and/or SCR2 epitopes of CD21. For example, ELISA with C3d-coated and soluble CD21-coated plates can be employed to determine which antibodies are specific for C3d binding portion of CD21 (i.e., SCRI and/or SCR2). Flow cytometry can be used to assess how well the antibody(-ies) bind to CD21-expressing cells, including but not limited to B cells or a cell line such as Raji. In the alternative, antibodies can be screened by combining with a population of B cells and then exposing the B cells to a source of C3d, either in isolated form (e.g. C3d coated plates) or in natural form (e.g., in serum). Flow cytometry and markers indicative of B cell activation, including but not limited to CD22, CD23, CD24, CD25, CD28, CD30, CD39, CD69, CD72, CD75, CD76, CD86, CD97, CD125, CD126, CD130, and CD153 can be used to detect how well the anti-CD21 antibody inhibits B cell activation.

As used herein, the term “Complementarity Determining Regions” (CDRs, i.e., CDR1, CDR2, and CDR3) refers to the amino acid residues of an antibody variable domain the presence of which are necessary for specific antigen binding. Each variable domain typically has three CDR regions identified as CDR1, CDR2 and CDR3. Each complementarity determining region can comprise amino acid residues from a “complementarity determining region” as defined by Kabat (i.e., about residues 24-34 (L1), 50-56 (L2) and 89-97 (L3) in the light chain variable domain and 31-35 (H1), 50-65 (H2) and 95-102 (H3) in the heavy chain variable domain). Likewise, “frameworks” (FWs) comprise amino acids 1-23 (FW1), 35-49 (FW2), 57-88 (FW3), and 98-107 (FW4) in the light chain variable domain and 1-30 (FW1), 36-49 (FW2), 66-94 (FW3), and 103-113 (FW4) in the heavy chain variable domain taking into account the Kabat numbering system (Kabat et al., Sequences of Proteins of Immunological Interest, 5th Ed. Public Health Service, National Institutes of Health, Bethesda, Md. (1987, 1991)).

The Kabat residue designations do not always correspond directly with the linear numbering of the amino acid residues. The actual linear amino acid sequence can contain fewer or additional amino acids than in the strict Kabat numbering corresponding to a shortening of, or insertion into, a structural component, whether framework or complementarity determining region (CDR), of the basic variable domain structure. The correct Kabat numbering of residues can be determined for a given antibody by alignment of residues of homology in the sequence of the antibody with a “standard” Kabat numbered sequence. Methods and computer programs for determining sequence similarity are publicly available, including, but not limited to, the GCG program package (Devereux et al., Nucleic Acids Research 12:387, 1984), BLASTP, BLASTN, FASTA (Altschul et al., J. Mol. Biol. 215:403 (1990), and the ALIGN program (version 2.0). The well-known Smith Waterman algorithm can also be used to determine similarity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). In comparing sequences, these methods account for various substitutions, deletions, and other modifications.

TABLE 1
Exemplary heavy chain (HC), light chain (LC) and CDR sequences
of exemplary CD21 antibodies as described herein.

| Antibody Chain | | SEQ ID |
|---|---|---|
| and/or CDR | Sequence | NO: |
| Ab15_H | QVQLVQSGAE VKKPGSSVKV SCKASGGTFS SYAISWVRQA | 1 |
| | PGQGLEWMGR IIPILGIANY AQKFQGRVTI TADKSTSTAY | |
| | MELSSLRSED TAVYYCARED DSSGYYQDWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPEAAGAP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSREEM | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPGK | |
| Ab15_H_CDR_H1 | GGTFSSYAIS | 2 |
| Ab15_H_CDR_H2 | RIIPILGIAN YAQKFQG | 3 |
| Ab15_H_CDR_H3 | EDDSSGYYQD | 4 |
| Ab15_H_CH1 | ASTKGPSVFP LAPSSKSTSG GTAALGCLVK DYFPEPVTVS | 5 |
| | WNSGALTSGV HTFPAVLQSS GLYSLSSVVT VPSSSLGTQT | |
| | ICNVNHKPS NTKVDKKV | |
| Ab15_H | EPKSCDKTHT CPPCP | 6 |
| CH1_CH2_HINGE | | |
| Ab15_H_CH2 | APEAAGAPSV FLFPPKPKDT LMISRTPEVT CVVVDVSHED | 7 |
| | PEVKFNWYVD GVEVHNAKTK PREEQYNSTY RVVSVLTVLH | |
| | QDWLNGKEYK CKVSNKALPA PIEKTISKAK | |
| Ab15_H_CH3 | GQPREPQVYT LPPSREEMTK NQVSLTCLVK GFYPSDIAVE | 8 |
| | WESNGQPENN YKTTPPVLDS DGSFFLYSKL TVDKSRWQQG | |
| | NVFSCSVMHE ALHNHYTQKS LSLSPGK | |
| Ab15_H_FV_H | QVQLVQSGAE VKKPGSSVKV SCKASGGTFS SYAISWVRQA | 9 |
| | PGQGLEWMGR IIPILGIANY AQKFQGRVTI TADKSTSTAY | |
| | MELSSLRSED TAVYYCARED DSSGYYQDWG QGTLVTVSS | |
| Ab15_H_FW_H1 | QVQLVQSGAE VKKPGSSVKV SCKAS | 10 |
| Ab15_H_FW_H2 | WVRQAPGQGL EWMG | 11 |
| Ab15_H_FW_H3 | RVTITADKST STAYMELSSL RSEDTAVYYC AR | 12 |
| Ab15_H_FW_H4 | WGQGTLVTVS S | 13 |
| Ab15_L | EIVMTQSPAT LSLSPGESAT LSCRAGQSIN SHLAWYQQKP | 14 |
| | GQSPSLLIYD ASTRATGVPA RFSGSGFGTE FTLTISSLQS | |
| | EDFAVYFCQQ YEYWYTFGQG TKLEIKRTVA APSVFIFPPS | |
| | DEQLKSGTAS VVCLLNNFYP REAKVQWKVD NALQSGNSQE | |
| | SVTEQDSKDS TYSLSSTLTL SKADYEKHKV YACEVTHQGL | |
| | SSPVTKSENR GEC | |
| Ab15_L_CDR_L1 | RAGQSINSHL A | |
| Ab15_L_CDR_L2 | DASTRAT | 16 |
| Ab15_L_CDR_L3 | QQYEYWYT | 17 |
| Ab15_L_CL | RTVAAPSVFI FPPSDEQLKS GTASVVCLLN NFYPREAKVQ | 18 |
| | WKVDNALQSG NSQESVTEQD SKDSTYSLSS TLTLSKADYE | |
| | KHKVYACEVT HQGLSSPVTK SFNRGEC | |
| Ab15_L_FV_L | EIVMTQSPAT LSLSPGESAT LSCRAGQSIN SHLAWYQQKP | 19 |
| | GQSPSLLIYD ASTRATGVPA RESGSGFGTE FTLTISSLQS | |
| | EDFAVYFCQQ YEYWYTFGQG TKLEIK | |
| Ab15_L_FW_LI | EIVMTQSPAT LSLSPGESAT LSC | 20 |
| Ab15_L_FW_L2 | WYQQKPGQSP SLLIY | 21 |
| Ab15_L_FW_L3 | GVPARFSGSG FGTEFTLTIS SLQSEDFAVY FC | 22 |
| Ab15_L_FW_L4 | FGQGTKLEIK | 23 |
| Ab89_L | EIVMTQSPAT LSVSPGERAT LSCRAGQSIN SHLAWYQQKP | 24 |
| | GQAPRLLIYD ASTRATGIPA RESGSGSGTE FTLTISSLQS | |
| | EDFAVYYCQQ YEYWYTFGQG TKLEIKRTVA APSVFIFPPS | |
| | DEQLKSGTAS VVCLLNNFYP REAKVQWKVD NALQSGNSQE | |
| | SVTEQDSKDS TYSLSSTLTL SKADYEKHKV YACEVTHQGL | |
| | SSPVTKSENR GEC | |
| Ab89_L_FV_L | EIVMTQSPAT LSVSPGERAT LSCRAGQSIN SHLAWYQQKP | 25 |
| | GQAPRLLIYD ASTRATGIPA RESGSGSGTE FTLTISSLQS | |
| | EDFAVYYCQQ YEYWYTFGQG TKLEIK | |
| Ab89_L_FW_LI | EIVMTQSPAT LSVSPGERAT LSC | 26 |
| Ab89_L_FW_L2 | WYQQKPGQAP RLLIY | 27 |
| Ab89_L_FW_L3 | GIPARFSGSG SGTEFTLTIS SLQSEDFAVY YC | 28 |
| Ab194_H | QVQLVQSGAE VKKPGSSVKV SCKASGGTFS TYAISWVRQA | 29 |
| | PGQGLEWMGR IIPILGIANY AQKFQGRVTI TADKSTSTAY | |
| | MELSSLRSED TAVYYCARED DSSGYYQDWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPEAAGAP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSREEM | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPGK | |
| Ab194_H_CDR_H1 | GGTESTYAIS | 30 |
| Ab194_H_FV_H | QVQLVQSGAE VKKPGSSVKV SCKASGGTES TYAISWVRQA | 31 |
| | PGQGLEWMGR IIPILGIANY AQKFQGRVTI TADKSTSTAY | |
| | MELSSLRSED TAVYYCARED DSSGYYQDWG QGTLVTVSS | |
| Ab194_L | EIVMTQSPAT LSVSPGERAT LSCRAGQSIN SYLAWYQQKP | 32 |
| | GQAPRLLIYD ASTRATGIPA RESGSGSGTE FTLTISSLQS | |
| | EDFAVYYCQQ YEYWYTFGQG TKLEIKRTVA APSVFIFPPS | |
| | DEQLKSGTAS VVCLLNNFYP REAKVQWKVD NALQSGNSQE | |
| | SVTEQDSKDS TYSLSSTLTL SKADYEKHKV YACEVTHQGL | |
| | SSPVTKSENR GEC | |
| Ab194_L_CDR_L1 | RAGQSINSYL A | 33 |
| Ab194_L_FV_L | EIVMTQSPAT LSVSPGERAT LSCRAGQSIN SYLAWYQQKP | 34 |
| | GQAPRLLIYD ASTRATGIPA RFSGSGSGTE FTLTISSLQS | |
| | EDFAVYYCQQ YEYWYTFGQG TKLEIK | |
| Ab204_H | QVQLVQSGAE VKKPGSSVKV SCKASGGELQ DLAISWVRQA | 35 |
| | PGQGLEWMGR IIPILGIANY AQKFQGRVTI TADKSTSTAY | |
| | MELSSLRSED TAVYYCARED DISGYYQDWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPEAAGAP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSREEM | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPGK | |
| Ab204_H_CDR_H1 | GGELQDLAIS | 36 |
| Ab204_H_CDR_H3 | EDDISGYYQD | 37 |
| Ab204_H_FV_H | QVQLVQSGAE VKKPGSSVKV SCKASGGELQ DLAISWVRQA | 38 |
| | PGQGLEWMGR IIPILGIANY AQKFQGRVTI TADKSTSTAY | |
| | MELSSLRSED TAVYYCARED DISGYYQDWG QGTLVTVSS | |
| Ab414_H | QVQLVQSGAE VKKPGSSVKV SCKASGGTES TYAISWVRQA | 39 |
| | PGQGLEWMGR IIPILGIANY AQKFQGRVTI TADKSTSTAY | |
| | MELSSLRSED TAVYYCARED DSSGYYQDWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPEAAGAP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSREEM | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPG | |
| Ab414_H_CH3 | GQPREPQVYT LPPSREEMTK NQVSLTCLVK GFYPSDIAVE | 40 |
| | WESNGQPENN YKTTPPVLDS DGSFFLYSKL TVDKSRWQQG | |
| | NVFSCSVMHE ALHNHYTQKS LSLSPG | |
| Ab416_H | QVQLVQSGAE VKKPGSSVKV SCKASGGTES SYAISWVRQA | 41 |
| | PGQGLEWMGR IIPILGIANY AQKFQGRVTI TADKSTSTAY | |
| | MELSSLRSED TAVYYCARED DSSGYYQDWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPEAAGAP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSREEM | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPG | |
| Ab417_H | QVQLVQSGAE VKKPGSSVKV SCKASGGELQ DLAISWVRQA | 42 |
| | PGQGLEWMGR IIPILGIANY AQKFQGRVTI TADKSTSTAY | |
| | MELSSLRSED TAVYYCARED DISGYYQDWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPEAAGAP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSREEM | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPG | |
| Ab419_H | QVQLVQSGAE VKKPGSSVKV SCKASGGELQ TLAISWVRQA | 43 |
| | PGQGLEWMGR IIPILGIANY AQKFQGRVTI TADKSTSTAY | |
| | MELSSLRSED TAVYYCARED DISGYYQDWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPEAAGAP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSREEM | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPG | |
| Ab419_H_CDR_H1 | GGELQTLAIS | 44 |
| Ab419_H_FV_H | QVQLVQSGAE VKKPGSSVKV SCKASGGELQ TLAISWVRQA | 45 |
| | PGQGLEWMGR IIPILGIANY AQKFQGRVTI TADKSTSTAY | |
| | MELSSLRSED TAVYYCARED DISGYYQDWG QGTLVTVSS | |
| Ab420_H | QVQLVQSGAE VKKPGSSVKV SCKASGGTFS SYAISWVRQA | 46 |
| | PGQGLEWMGR IIPILGIANY AQKFQGRVTI TADKSTSTAY | |
| | MELSSLRSED TAVYYCARED DISGYYQDWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPEAAGAP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSREEM | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPG | |
| Ab420_H_FV_H | QVQLVQSGAE VKKPGSSVKV SCKASGGTFS SYAISWVRQA | 47 |
| | PGQGLEWMGR IIPILGIANY AQKFQGRVTI TADKSTSTAY | |
| | MELSSLRSED TAVYYCARED DISGYYQDWG QGTLVTVSS | |
| Ab421_H | QVQLVQSGAE VKKPGSSVKV SCKASGGTES TYAISWVRQA | 48 |
| | PGQGLEWMGR IIPILGIANY AQKFQGRVTI TADKSTSTAY | |
| | MELSSLRSED TAVYYCARED DISGYYQDWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPEAAGAP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSREEM | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPG | |
| Ab421_H_FV_H | QVQLVQSGAE VKKPGSSVKV SCKASGGTFS TYAISWVRQA | 49 |
| | PGQGLEWMGR IIPILGIANY AQKFQGRVTI TADKSTSTAY | |
| | MELSSLRSED TAVYYCARED DISGYYQDWG QGTLVTVSS | |
| Ab626_H | QVQLVQSGAE VKKPGSSVKV SCKASGGTFS SYAISWVRQA | 50 |
| | PGQGLEWMGR IIPILGIANY AQKFQGRVTI TADKSTSTAY | |
| | MELSSLRSED TAVYYCAREE DISGYYQDWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPEAAGAP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSREEM | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPG | |
| Ab626_H_CDR_H3 | EEDISGYYQD | 51 |
| Ab626_H_FV_H | QVQLVQSGAE VKKPGSSVKV SCKASGGTFS SYAISWVRQA | 52 |
| | PGQGLEWMGR IIPILGIANY AQKFQGRVTI TADKSTSTAY | |
| | MELSSLRSED TAVYYCAREE DISGYYQDWG QGTLVTVSS | |
| Ab626_L | DIVMTQSPAT LSVSPGERAT LSCRAGQSIS SYLAWYQQKP | 53 |
| | GQAPRLLIYD ASTRATGIPA RESGSGSGTE FTLTISSLQS | |
| | EDFAVYYCQQ YEYWYSFGQG TKLEIKRTVA APSVFIFPPS | |
| | DEQLKSGTAS VVCLLNNFYP REAKVQWKVD NALQSGNSQE | |
| | SVTEQDSKDS TYSLSSTLTL SKADYEKHKV YACEVTHQGL | |
| | SSPVTKSENR GEC | |
| Ab626_L_CDR_L1 | RAGQSISSYL A | 54 |
| Ab626_L_CDR_L3 | QQYEYWYS | 55 |
| Ab626_L_FV_L | DIVMTQSPAT LSVSPGERAT LSCRAGQSIS SYLAWYQQKP | 56 |
| | GQAPRLLIYD ASTRATGIPA RFSGSGSGTE FTLTISSLQS | |
| | EDFAVYYCQQ YEYWYSFGQG TKLEIK | |
| Ab626_L_FW_LI | DIVMTQSPAT LSVSPGERAT LSC | 57 |
| Ab627_L | EIVMTQSPAT LSVSPGERAT LSCRAGQKIS SYLAWYQQKP | 58 |
| | GQAPRLLIYD ASTRATGIPA RESGSGSGTE FTLTISSLQS | |
| | EDFAVYYCQQ YEYWYSFGQG TKLEIKRTVA APSVFIFPPS | |
| | DEQLKSGTAS VVCLLNNFYP REAKVQWKVD NALQSGNSQE | |
| | SVTEQDSKDS TYSLSSTLTL SKADYEKHKV YACEVTHQGL | |
| | SSPVTKSENR GEC | |
| Ab627_L_CDR_L1 | RAGQKISSYL A | 59 |
| Ab627_L_FV_L | EIVMTQSPAT LSVSPGERAT LSCRAGQKIS SYLAWYQQKP | 60 |
| | GQAPRLLIYD ASTRATGIPA RESGSGSGTE FTLTISSLQS | |
| | EDFAVYYCQQ YEYWYSFGQG TKLEIK | |
| Ab628_L | EIVMTQSPAT LSVSPGERAT LSCRAGQSIS HYLAWYQQKP | 61 |
| | GQAPRLLIYD ASTRATGIPA RFSGSGSGTE FTLTISSLQS | |
| | EDFAVYYCQQ YEYWYSFGQG TKLEIKRTVA APSVFIFPPS | |
| | DEQLKSGTAS VVCLLNNFYP REAKVQWKVD NALQSGNSQE | |
| | SVTEQDSKDS TYSLSSTLTL SKADYEKHKV YACEVTHQGL | |
| | SSPVTKSENR GEC | |
| Ab628_L_CDR_L1 | RAGQSISHYL A | 62 |
| Ab628_L_FV_L | EIVMTQSPAT LSVSPGERAT LSCRAGQSIS HYLAWYQQKP | 63 |
| | GQAPRLLIYD ASTRATGIPA RFSGSGSGTE FTLTISSLQS | |
| | EDFAVYYCQQ YEYWYSFGQG TKLEIK | |
| Ab629_L | EIVMTQSPAT LSVSPGERAT LSCRAGQSIS RYLAWYQQKP | 64 |
| | GQAPRLLIYD ASTRATGIPA RFSGSGSGTE FTLTISSLQS | |
| | EDFAVYYCQQ YEYWYSFGQG TKLEIKRTVA APSVFIFPPS | |
| | DEQLKSGTAS VVCLLNNFYP REAKVQWKVD NALQSGNSQE | |
| | SVTEQDSKDS TYSLSSTLTL SKADYEKHKV YACEVTHQGL | |
| | SSPVTKSENR GEC | |
| Ab629_L_CDR_L1 | RAGQSISRYL A | 65 |
| Ab629_L_FV_L | EIVMTQSPAT LSVSPGERAT LSCRAGQSIS RYLAWYQQKP | 66 |
| | GQAPRLLIYD ASTRATGIPA RESGSGSGTE FTLTISSLQS | |
| | EDFAVYYCQQ YEYWYSFGQG TKLEIK | |
| Ab630_L | DIVMTQSPAT LSVSPGERAT LSCRAGQSIS SYLAWYQQKP | 67 |
| | GQAPRLLIYD ASSRATGIPA RFSGSGSGTE FTLTISSLQS | |
| | EDFAVYYCQQ YEYWYSFGQG TKLEIKRTVA APSVFIFPPS | |
| | DEQLKSGTAS VVCLLNNFYP REAKVQWKVD NALQSGNSQE | |
| | SVTEQDSKDS TYSLSSTLTL SKADYEKHKV YACEVTHQGL | |
| | SSPVTKSENR GEC | |
| Ab630_L_CDR_L2 | DASSRAT | 68 |
| Ab630_L_FV_L | DIVMTQSPAT LSVSPGERAT LSCRAGQSIS SYLAWYQQKP | 69 |
| | GQAPRLLIYD ASSRATGIPA RFSGSGSGTE FTLTISSLQS | |
| | EDFAVYYCQQ YEYWYSFGQG TKLEIK | |
| Ab631_L | EIVMTQSPAT LSVSPGERAT LSCRAGQKIS SYLAWYQQKP | 70 |
| | GQAPRLLIYD ASSRATGIPA RFSGSGSGTE FTLTISSLQS | |
| | EDFAVYYCQQ YEYWYSFGQG TKLEIKRTVA APSVFIFPPS | |
| | DEQLKSGTAS VVCLLNNFYP REAKVQWKVD NALQSGNSQE | |
| | SVTEQDSKDS TYSLSSTLTL SKADYEKHKV YACEVTHQGL | |
| | SSPVTKSFNR GEC | |
| Ab631_L_FV_L | EIVMTQSPAT LSVSPGERAT LSCRAGQKIS SYLAWYQQKP | 71 |
| | GQAPRLLIYD ASSRATGIPA RESGSGSGTE FTLTISSLQS | |
| | EDFAVYYCQQ YEYWYSFGQG TKLEIK | |
| Ab632_L | EIVMTQSPAT LSVSPGERAT LSCRAGQSIS HYLAWYQQKP | 72 |
| | GQAPRLLIYD ASSRATGIPA RESGSGSGTE FTLTISSLQS | |
| | EDFAVYYCQQ YEYWYSFGQG TKLEIKRTVA APSVFIFPPS | |
| | DEQLKSGTAS VVCLLNNFYP REAKVQWKVD NALQSGNSQE | |
| | SVTEQDSKDS TYSLSSTLTL SKADYEKHKV YACEVTHQGL | |
| | SSPVTKSENR GEC | |
| Ab632_L_FV_L | EIVMTQSPAT LSVSPGERAT LSCRAGQSIS HYLAWYQQKP | 73 |
| | GQAPRLLIYD ASSRATGIPA RESGSGSGTE FTLTISSLQS | |
| | EDFAVYYCQQ YEYWYSFGQG TKLEIK | |
| Ab633_L | EIVMTQSPAT LSVSPGERAT LSCRAGQSIS RYLAWYQQKP | 74 |
| | GQAPRLLIYD ASSRATGIPA RFSGSGSGTE FTLTISSLQS | |
| | EDFAVYYCQQ YEYWYSFGQG TKLEIKRTVA APSVFIFPPS | |
| | DEQLKSGTAS VVCLLNNFYP REAKVQWKVD NALQSGNSQE | |
| | SVTEQDSKDS TYSLSSTLTL SKADYEKHKV YACEVTHQGL | |
| | SSPVTKSENR GEC | |
| Ab633_L_FV_L | EIVMTQSPAT LSVSPGERAT LSCRAGQSIS RYLAWYQQKP | 75 |
| | GQAPRLLIYD ASSRATGIPA RFSGSGSGTE FTLTISSLQS | |
| | EDFAVYYCQQ YEYWYSFGQG TKLEIK | |
| Ab646_H | QVQLVQSGAE VKKPGSSVKV SCKASGGELQ TLAISWVRQA | 76 |
| | PGQGLEWMGR IIPILGIANY AQKFQGRVTI TADKSTSTAY | |
| | MELSSLRSED TAVYYCAREE DISGYYQDWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPEAAGAP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSREEM | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPG | |
| Ab646_H_FV_H | QVQLVQSGAE VKKPGSSVKV SCKASGGELQ TLAISWVRQA | 77 |
| | PGQGLEWMGR IIPILGIANY AQKFQGRVTI TADKSTSTAY | |
| | MELSSLRSED TAVYYCAREE DISGYYQDWG QGTLVTVSS | |
| Ab646_L | EIVMTQSPAT LSVSPGERAT LSCRAGQSIS SYLAWYQQKP | 78 |
| | GQAPRLLIYD ASSRATGIPA RESGSGSGTE FTLTISSLQS | |
| | EDFAVYYCQQ YEYWYSFGQG TKLEIKRTVA APSVFIFPPS | |
| | DEQLKSGTAS VVCLLNNFYP REAKVQWKVD NALQSGNSQE | |
| | SVTEQDSKDS TYSLSSTLTL SKADYEKHKV YACEVTHQGL | |
| | SSPVTKSENR GEC | |
| Ab646_L_FV_L | EIVMTQSPAT LSVSPGERAT LSCRAGQSIS SYLAWYQQKP | 79 |
| | GQAPRLLIYD ASSRATGIPA RFSGSGSGTE FTLTISSLQS | |
| | EDFAVYYCQQ YEYWYSFGQG TKLEIK | |
| Ab647_H | QVQLVQSGAE VKKPGSSVKV SCKASGGELQ TYAISWVRQA | 80 |
| | PGQGLEWMGR IIPILGIANY AQKFQGRVTI TADKSTSTAY | |
| | MELSSLRSED TAVYYCAREE DISGYYQDWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPEAAGAP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSREEM | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPG | |
| Ab647_H_CDR_H1 | GGELQTYAIS | 81 |
| Ab647_H_FV_H | QVQLVQSGAE VKKPGSSVKV SCKASGGELQ TYAISWVRQA | 82 |
| | PGQGLEWMGR IIPILGIANY AQKFQGRVTI TADKSTSTAY | |
| | MELSSLRSED TAVYYCAREE DISGYYQDWG QGTLVTVSS | |
| Ab648_H | QVQLVQSGAE VKKPGSSVKV SCKASGGELS TYAISWVRQA | 83 |
| | PGQGLEWMGR IIPILGIANY AQKFQGRVTI TADKSTSTAY | |
| | MELSSLRSED TAVYYCAREE DISGYYQDWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPEAAGAP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSREEM | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPG | |
| Ab648_H_CDR_H1 | GGELSTYAIS | 84 |
| Ab648_H_FV_H | QVQLVQSGAE VKKPGSSVKV SCKASGGELS TYAISWVRQA | 85 |
| | PGQGLEWMGR IIPILGIANY AQKFQGRVTI TADKSTSTAY | |
| | MELSSLRSED TAVYYCAREE DISGYYQDWG QGTLVTVSS | |
| Ab649_H | QVQLVQSGAE VKKPGSSVKV SCKASGGEFQ SYAISWVRQA | 86 |
| | PGQGLEWMGR IIPILGIANY AQKFQGRVTI TADKSTSTAY | |
| | MELSSLRSED TAVYYCAREE DISGYYQDWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPEAAGAP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSREEM | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPG | |
| Ab649_H_CDR_H1 | GGEFQSYAIS | 87 |
| Ab649_H_FV_H | QVQLVQSGAE VKKPGSSVKV SCKASGGEFQ SYAISWVRQA | 88 |
| | PGQGLEWMGR IIPILGIANY AQKFQGRVTI TADKSTSTAY | |
| | MELSSLRSED TAVYYCAREE DISGYYQDWG QGTLVTVSS | |
| Ab650_H | QVQLVQSGAE VKKPGSSVKV SCKASGGTFQ TYAISWVRQA | 89 |
| | PGQGLEWMGR IIPILGIANY AQKFQGRVTI TADKSTSTAY | |
| | MELSSLRSED TAVYYCAREE DISGYYQDWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPEAAGAP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSREEM | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPG | |
| Ab650_H_CDR_H1 | GGTFQTYAIS | 90 |
| Ab650_H_FV_H | QVQLVQSGAE VKKPGSSVKV SCKASGGTFQ TYAISWVRQA | 91 |
| | PGQGLEWMGR IIPILGIANY AQKFQGRVTI TADKSTSTAY | |
| | MELSSLRSED TAVYYCAREE DISGYYQDWG QGTLVTVSS | |
| Ab651_H | QVQLVQSGAE VKKPGSSVKV SCKASGGTLS TYAISWVRQA | 92 |
| | PGQGLEWMGR IIPILGIANY AQKFQGRVTI TADKSTSTAY | |
| | MELSSLRSED TAVYYCAREE DISGYYQDWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPEAAGAP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSREEM | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPG | |
| Ab651_H_CDR_H1 | GGTLSTYAIS | 93 |
| Ab651_H_FV_H | QVQLVQSGAE VKKPGSSVKV SCKASGGTLS TYAISWVRQA | 94 |
| | PGQGLEWMGR IIPILGIANY AQKFQGRVTI TADKSTSTAY | |
| | MELSSLRSED TAVYYCAREE DISGYYQDWG QGTLVTVSS | |
| Ab652_H | QVQLVQSGAE VKKPGSSVKV SCKASGGELS SYAISWVRQA | 95 |
| | PGQGLEWMGR IIPILGIANY AQKFQGRVTI TADKSTSTAY | |
| | MELSSLRSED TAVYYCAREE DISGYYQDWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPEAAGAP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSREEM | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPG | |
| Ab652_H_CDR_H1 | GGELSSYAIS | 96 |
| Ab652_H_FV_H | QVQLVQSGAE VKKPGSSVKV SCKASGGELS SYAISWVRQA | 97 |
| | PGQGLEWMGR IIPILGIANY AQKFQGRVTI TADKSTSTAY | |
| | MELSSLRSED TAVYYCAREE DISGYYQDWG QGTLVTVSS | |
| Ab653_H | QVQLVQSGAE VKKPGSSVKV SCKASGGEFS TYAISWVRQA | 98 |
| | PGQGLEWMGR IIPILGIANY AQKFQGRVTI TADKSTSTAY | |
| | MELSSLRSED TAVYYCAREE DISGYYQDWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPEAAGAP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSREEM | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPG | |
| Ab653_H_CDR_H1 | GGEFSTYAIS | 99 |
| Ab653_H_FV_H | QVQLVQSGAE VKKPGSSVKV SCKASGGEFS TYAISWVRQA | 100 |
| | PGQGLEWMGR IIPILGIANY AQKFQGRVTI TADKSTSTAY | |
| | MELSSLRSED TAVYYCAREE DISGYYQDWG QGTLVTVSS | |
| Ab658_H | QVQLVQSGAE VKKPGSSVKV SCKASGGELS SYAISWVRQA | 101 |
| | PGQGLEWMGR IIPILGIANY AQKFQGRVTI TADKSTSTAY | |
| | MELSSLRSED TAVYYCAREE DISGYYQDWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPEAAGAP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSREEM | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPGK | |

TABLE 2
Heavy Chain and Light Chain CDR Consensus
Sequences for CD21 binding proteins

| | Consensus Sequence | SEQ |
|---|---|---|
| | | ID |
| | | NO: |
| CDR1 | G-G-X₁-X₂-X₃-X₄-X₅-A-I-S | 102 |
| (Heavy Chain) | wherein X₁ is T or E; | |
| | X₂ is F or L; X₃ is S, | |
| | or Q; X₄ is T, | |
| | D, or S; | |
| | X₅ is Y or L | |
| CDR3 | E-X₆-D-X₇-S-G-Y-Y-Q-D | 103 |
| (Heavy Chain) | wherein X₆ is D or E; | |
| | X₇ is S or I | |
| CDR1 | R-A-G-Q-X₈-I-X₉- | 104 |
| (Light Chain) | X₁₀-Y-L-A | |
| | wherein X8 is S or K; | |
| | X₉ is N or S; | |
| | X₁₀ is S, H, or R | |
| CDR2 | D-A-S-X₁₁-R-A-T | 105 |
| (Light Chain) | wherein X₁₁ is S or T | |
| CDR3 | Q-Q-Y-E-Y-W-Y-X₁₂ | 106 |
| (Light Chain) | wherein X₁₂ is S or T | |

Immunoglobin CD21 binding proteins (e.g., IgG or Fab CD21 binding proteins) can be modified to reduce immunogenicity. Reduced immunogenicity is desirable in CD21 binding proteins intended for use as therapeutics, as it reduces the chance that the subject will develop an immune response against the therapeutic molecule. Techniques useful for reducing immunogenicity of CD21 binding proteins include deletion/modification of potential human T cell epitopes and ‘germlining’ of sequences outside of the CDRs (e.g., framework and Fc). Such methods are known to those of skill in the art and are not described in detail herein.

Standard recombinant nucleic acid methods can be used to express a protein that binds to CD21. Generally, a nucleic acid sequence encoding the protein is cloned into a nucleic acid expression vector. Of course, if the protein includes multiple polypeptide chains, each chain can be cloned into an expression vector, e.g., the same or different vectors, that are expressed in the same or different cells.

In certain embodiments, the antibodies described herein are bound to one or more carriers. Carriers can be active and/or inert. Examples of well-known carriers include polypropylene, polystyrene, polyethylene, dextran, nylon, amylases, glass, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

The antibodies can also be conjugated to a detectable agent. The complex is useful to detect the antigens to which the antibody specifically binds in a sample, using standard immunochemical techniques such as flow cytometry or immunohistochemistry as described by Harlow and Lane (1988) supra. Detectable markers can also be used to ascertain binding specificity for a type of cell (e.g., B cell) by using the detectable marker with another marker which is definitive for B cells (e.g., CD19, CD20, CD22, etc.) and analyzing the staining patterns by FACS. There are many different labels and methods of labeling known to those of ordinary skill in the art. Examples of the types of labels which can be used with the antibodies described herein include radioisotopes, enzymes, colloidal metals, fluorescent compounds (e.g., FITC, PE, PECy5, APC, etc.), bioluminescent compounds, and chemiluminescent compounds. Those of ordinary skill in the art will know of other suitable labels for binding to the antibody, or will be able to ascertain such, using routine experimentation. Furthermore, the binding of these labels to an antibody as described herein can be done using standard techniques common to those of ordinary skill in the art.

### Polynucleotides

The disclosure also provides a polynucleotide encoding an antibody or antigen-binding fragment thereof described herein. The skilled person will understand that, due to the degeneracy of the genetic code, a given antibody or antigen-binding fragment thereof can be encoded by different polynucleotides. These “variants” are encompassed herein.

In some embodiments, a nucleic acid encoding an antibody or antigen-binding fragment thereof described herein is comprised in a vector. In some embodiments, a nucleic acid sequence encoding an antibody or antigen-binding fragment thereof described herein is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments, the expression system is an organism other than the source of the native/wild-type sequence (or a cell obtained from such organism). In some embodiments, the vector and/or nucleic acid sequence described herein is codon-optimized for expression in a mammal or mammalian cell, e.g., a mouse, a murine cell, or a human cell. In some embodiments, the vector and/or nucleic acid sequence described herein is codon-optimized for expression in a human cell. In some embodiments, the vector and/or nucleic acid sequence described herein is codon-optimized for expression in a yeast or yeast cell. In some embodiments, the vector and/or nucleic acid sequence described herein is codon-optimized for expression in a bacterial cell. In some embodiments, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding an antibody or antigen-binding fragment thereof as described herein in place of non-essential viral genes. The vector and/or particle may be utilized for the purpose of transferring any nucleic acids into cells either in vitro or in vivo. Numerous forms of viral vectors are known in the art.

### Cells

The disclosure also provides a cell comprising an antibody or an antigen binding fragment described herein. The disclosure further provides a host cell comprising a polynucleotide described herein or a plasmid or vector described herein. As used herein, the term “cell” refers to a single cell as well as to a population of (i.e., more than one) cells.

A host cell can be a prokaryotic or eukaryotic host cell. Exemplary host cells include, but are not limited to, bacterial cells, yeast cells, plant cell, animal (including insect) or human cells. The host cells can be employed in a method of producing an antibody or antigen-binding fragment thereof described herein. Generally, the method comprises: culturing a host cell comprising a polynucleotide described herein or a plasmid or vector described herein under conditions such that the antibody or antigen-binding fragment thereof is expressed; and optionally recovering the antibody or antigen-binding fragment thereof from the culture medium. The antibody or antigen-binding fragment thereof can be concentrated and purified by a variety of biochemical and chromatographic methods, including methods utilizing differences in size, charge, hydrophobicity, solubility, specific affinity, etc. between the antibody or antigen-binding fragment thereof and other substances in the cell culture medium. In some embodiments, the antibody or antigen-binding fragment thereof is secreted from the host cells.

The antibody or antigen-binding fragment thereof described herein can be produced as recombinant molecules in prokaryotic or eukaryotic host cells, such as bacteria, yeast, plant, animal (including insect) or human cell lines or in transgenic animals. Recombinant methods of producing a polypeptide through the introduction of a vector including nucleic acid encoding the polypeptide into a suitable host cell is well known in the art, such as is described in Sambrook et al., Molecular Cloning: A Laboratory Manual, 2d Ed, Vols 1 to 8, Cold Spring Harbor, NY (1989); M. W. Pennington and B. M. Dunn, Methods in Molecular Biology: Peptide Synthesis Protocols, Vol 35, Humana Press, Totawa, NJ (1994), contents of both of which are herein incorporated by reference.

### Administering to a Cell

The antibody of antigen binding fragment described herein can inhibit or reduce CD21 activity or CD21/C3d mediated B cell activation. Accordingly, an antibody or antigen binding fragment described herein, or a polynucleotide encoding the same, can be administered to a cell, e.g., a B cell for inhibiting or reducing CD21 activity or CD21/C3d mediated B cell activation.

It is noted that administering to the cell can be in vitro or in-vivo. Methods for administering antibodies, antigen binding fragments and polynucleotides to a cell are well known and available to one of skill in the art. As used herein, administering to the cell means contacting the cell with the antibody or antigen binding fragment described herein, or a polynucleotide encoding the same, so that the antibody, the antigen binding fragment or the polynucleotide is taken up by the cell. Generally, the cell can be contacted with the antibody, the antigen binding fragment or the polynucleotide in a cell culture e.g., in vitro or ex vivo, or the antibody, the antigen binding fragment or the polynucleotide can be administrated to a subject, e.g., in vivo. The term “contacting” or “contact” as used herein in connection with contacting a cell includes subjecting the cells to an appropriate culture media, which comprises the antibody, the antigen binding fragment or the polynucleotide. Where the cell is in vivo, “contacting” or “contact” includes administering the compound, e.g., in a pharmaceutical composition to a subject via an appropriate administration route such that the compound contacts the cell in vivo.

For example, when the cell is in vitro, said administering to the cell can include subjecting the cell to an appropriate culture media which comprises the antibody, the antigen binding fragment or the polynucleotide. Where the cell is in vivo, said administering to the cell includes administering the antibody, the antigen binding fragment or the polynucleotide to a subject via an appropriate administration route such that the the antibody, the antigen binding fragment or the polynucleotide is administered to the cell in vivo.

### Compositions, Formulations and Packaging

Also provided herein are compositions, including pharmaceutical compositions, comprising an antibody or antigen-binding fragment that binds CD21 as described herein or a polynucleotide encoding the antibody or antigen-binding fragment. In one embodiment, the compositions are pharmaceutical compositions. Pharmaceutical compositions for use with the methods described herein can be formulated in a conventional manner using one or more physiologically acceptable carriers or excipients. Thus, the compounds and their physiologically acceptable salts and solvates can be formulated for administration by, for example, by aerosol, intravenous, oral or topical route. The compositions can be formulated for intralesional, intratumoral, intraperitoneal, subcutaneous, intramuscular or intravenous injection; infusion; liposome-mediated delivery; topical, intrathecal, gingival pocket, per rectum, intrabronchial, nasal, transmucosal, intestinal, oral, ocular or otic delivery.

Techniques and formulations generally can be found in Remmington's Pharmaceutical Sciences, Meade Publishing Co., Easton, PA. For systemic administration, injection is preferred, including intramuscular, intravenous, intraperitoneal, and subcutaneous. For injection, the anti-CD21 antibodies described herein can be formulated in liquid solutions, preferably in physiologically compatible buffers such as Hank's solution or Ringer's solution. In addition, the antibodies can be formulated in solid form and redissolved or suspended immediately prior to use. Lyophilized forms are also included.

For oral administration, the pharmaceutical composition can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they may be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., pharmaceutically acceptable oils, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound. For buccal administration the compositions can take the form of tablets or lozenges formulated in conventional manner. For administration by inhalation, the compounds for use as described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The anti-CD21 antibodies can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

In addition to the formulations described previously, the CD21 antibodies or antigen-binding fragments thereof (or the polynucleotide encoding the antibody or antigen-binding fragment) can also be formulated as a depot preparation. Such long acting formulations can be administered by implantation (for example subcutaneously or intramuscularly) or by intramuscular injection. Thus, for example, the antibodies can be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents can be used to facilitate permeation. Transmucosal administration can be through nasal sprays or using suppositories. For topical administration, the CD21 antibodies can be formulated into ointments, salves, gels, or creams as generally known in the art. A wash solution can be used locally to treat an injury or inflammation to accelerate healing.

The compositions can, if desired, be presented in a pack or dispenser device which can contain one or more-unit dosage forms containing the active ingredient. The pack can for example comprise metal or plastic foil, such as a blister pack. The pack or dispenser device can be accompanied by instructions for administration.

### Dosage and Administration

The method and compositions provided herein can be used to treat an autoimmune disease in a subject by administering a therapeutically effective amount of an antibody or antigen-binding fragment thereof that binds CD21. In one embodiment, the subject can be a mammal. In another embodiment, the mammal can be a human, although the approach is effective with respect to all mammals.

The appropriate dosage range for a CD21 antibody (or antigen-binding fragment thereof) depends upon the potency of the agent, and includes amounts large enough to produce the desired effect, e.g., reduction in at least one sign or symptom of an autoimmune disease or disorder. The dosage should not be so large as to cause unacceptable or life-threatening adverse side effects. Generally, the dosage will vary with the type of antibody or antigen-binding fragment, and with the age, condition, and sex of the patient. The dosage can be determined by one of skill in the art and can also be adjusted by the individual physician in the event of any complication.

The effective amount can be based upon, among other things, the specificity of the antibody, the size of the antibody, the biodegradability of the antibody, the bioactivity and/or the bioavailability of the antibody or antigen binding fragment. For example, if the antibody does not degrade quickly, is bioavailable and highly active, a smaller amount will be required to be effective. One of skill in the art can routinely perform empirical activity tests to determine the bioactivity in bioassays and thus determine the effective amount.

Typically, the dosage ranges for a CD21 antibody or fragment thereof are in the range of 0.001 mg/kg body weight to 5 g/kg body weight. In some embodiments, the dosage range is from 0.001 mg/kg body weight to 1g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 0.5 μg/mL and 30 μg/mL. Dosages for monoclonal antibodies can also be based on rational dosage predictions such as those described in Bai et al. “A Guide to Rational Dosing of Monoclonal Antibodies” Clinical Pharmacokinetics 51:119-135 (2012).

As one of skill in the art will appreciate, the dosage of an anti-CD21 antibody or antigen-binding fragment can vary depending upon the dosage form employed and the route of administration utilized. Compositions, methods, and uses that exhibit large therapeutic indices (i.e., the dose ration between toxic and therapeutic effects) are preferred. A therapeutically effective dose can be estimated initially from cell culture assays. Also, a dose can be formulated in animal models to achieve a circulating plasma concentration range that includes the IC50, which achieves a half-maximal inhibition of measured function or activity as determined in cell culture, or in an appropriate animal model. The effects of any particular dosage can be monitored by a suitable bioassay. A therapeutically effective amount is an amount of an anti-CD21 agent that is sufficient to produce a statistically significant, measurable change of a given symptom of an autoimmune disease or disorder (see “Efficacy Measurement” below). Such effective amounts can also be gauged in clinical trials as well as animal studies for a given agent.

An appropriate therapeutic amount or dose for treating a human subject can be informed by data collected in cell cultures or animal models. In some embodiments, the therapeutic efficacy can be estimated by the ED50 in an animal model (the dose therapeutically effective in 50% of the population). Murine models of autoimmune diseases (e.g., SLE) can be used, in part, to inform one of skill in the art with respect to potency of a given antibody, the dosage range needed for efficacy, and toxic doses. An exemplary spontaneous lupus murine model comprises female New Zealand Black×New Zealand White (NZB×NZW) F1 mice. Another model which can be utilized is the MRL 1pr/1pr spontaneous lupus model. MRL 1pr/1pr mice develop symptoms which are similar to human with SLE. These symptoms include but are not limited to high titer anti-dsDNA antibodies, hypocomplementemia, lymphadenopathy, and fatal immune complex-mediated glomerulonephritis.

Therapeutic compositions can be conventionally administered in a unit dose. The term “unit dose” when used in reference to a therapeutic composition refers to physically discrete units suitable as unitary dosage for the subject, each unit containing a predetermined quantity of an anti-cancer agent calculated to produce the desired therapeutic effect in association with the required physiologically acceptable diluent, i.e., carrier, or vehicle.

Administration of the doses recited above or as employed by a skilled clinician can be repeated for a limited and defined period of time. In some embodiments, the doses are given once a day, or multiple times a day (e.g., at least two times a day, at least three times a day etc). In a preferred embodiment, the doses recited above are administered daily for several weeks or months. The duration of treatment depends upon the subject's clinical progress and continued responsiveness to therapy. Continuous, relatively low maintenance doses are contemplated after an initial higher therapeutic dose. In one embodiment of the methods described herein, the anti-CD21 agent is administered at least once per day. In one embodiment, the anti-CD21 agent is administered daily. In one embodiment, the CD21 antibody or antigen-binding fragment thereof is administered every other day. In one embodiment, the anti-CD21 agent is administered every 6 to 8 days. In one embodiment, the agent is administered weekly.

Administration of an antibody or fragment thereof that binds CD21 can be constant for a certain period of time or periodic and at specific intervals. The anti-CD21 agent can be delivered hourly, daily, weekly, monthly, yearly (e.g. in a time release form) or as a one-time delivery. The delivery can be continuous delivery for a period of time, e.g. intravenous delivery.

The agents described herein can be administered to a subject in need thereof by any appropriate route which results in an effective treatment in the subject. For example, agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. The agent can be administered systemically, if so desired.

In some embodiments, the CD21 antibody or antigen-binding fragment thereof can be administered to a subject by any mode of administration that delivers the agent systemically or locally to a desired surface or target, and can include, but is not limited to, injection, infusion, instillation, and inhalation administration. To the extent that polypeptide agents can be protected from inactivation in the gut, oral administration forms are also contemplated. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of the agents described herein, other than directly into a target site, tissue, or organ, such that it enters the subject's circulatory system and, thus, is subject to metabolism and other like processes.

It is noted that the terms “administered” and “subjected” are used interchangeably in the context of treatment of a disease or disorder. In jurisdictions that forbid the patenting of methods that are practiced on the human body, the meaning of “administering” of a composition to a human subject shall be restricted to prescribing a controlled substance that a human subject will be administer to the subject by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology, if so desired.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

### Efficacy

The efficacy of a given treatment for an autoimmune disease or disorder (e.g., lupus) can be determined by the skilled clinician. However, a treatment is considered “effective treatment,” as the term is used herein, if any one or all of the signs or symptoms of the autoimmune disease/disorder is/are altered in a beneficial manner, or other clinically accepted symptoms or markers of disease are improved, or ameliorated, e.g., by at least 10% following treatment with a CD21 antibody or antigen binding fragment thereof. Efficacy can also be measured by failure of an individual to worsen as assessed by stabilization of the disease, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the autoimmune disease or disorder; or (2) relieving the disease, e.g., causing regression of symptoms; and (3) preventing or reducing the likelihood of the development of the disease, or preventing secondary diseases/disorders associated with the autoimmune disease or disorder.

An effective amount for the treatment of a disease means that amount which, when administered to a mammal in need thereof, is sufficient to result in effective treatment as that term is defined herein, for that disease. Efficacy of an agent can be determined by assessing physical indicators of the disease, such as e.g., pain, inflammation, fever, fatigue etc.

Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example animal models of systemic lupus erythematosis, e.g. a murine model. When using an experimental animal model, efficacy of treatment is evidenced when a statistically significant change in a marker is observed.

Treatment of SLE or a symptom thereof includes any aspect of SLE, including, but not limited to, immunological disorders (e.g., positive lupus erythematosus cell preparation, anti-DNA antibody to native DNA in abnormal titer, anti-SM nuclear antigen antibodies, anti-β2GPI antibodies), rashes, photosensitivity, oral ulcers, arthritis, serositis (pleuritis and/or pericarditis), renal disorders (e.g., proteinuria), neurological disorders (e.g., seizures or psychosis), hematological disorders (e.g., hemolytic anemia, leukopenia, lymphopenia, thrombocytopenia, secondary thrombocytopenia purpura), or lupus nephritis, which is a chronic inflammatory kidney disease. During lupus, nephritis “flares” can occur. “Flares” refer to an increase in activity, generally inflammatory activity. If the activity is in the kidneys, then the flare is referred to as a “renal flare”. “Renal flares” can be identified by evaluating factors including, but not limited to, proteinuria levels, hematuria levels, and serum creatinine levels. In one embodiment, treatment of SLE includes prevention or SLE or a symptom thereof (e.g., lupus nephritis) by administration to a subject when no symptoms of SLE are present. Also encompassed by “treatment of lupus” or “treatment of SLE” is a reduction of pathological consequences of any aspect of lupus, such as lupus nephritis. In some embodiments, treatment of SLE can comprise reduction of autoreactive B cells, which in turn reduces the production of autoantibodies.

Treatment or prevention of thyroiditis includes any aspect of thyroiditis or a symptom thereof including, but not limited to, excessive infiltration with chronic inflammatory cells, follicular rupture, eosinophilia, varying degrees of hyperplasia, fibrosis, painless goiter, and hypothyroidism. Treatment of graft-vs-host disease includes treatment of any aspect of graft vs. host disease including, but not limited to, reduction or elimination of foreign tissue rejection, lowering of antibody titers to the transplanted tissue, and reduction or elimination of graft-versus-host responses. Treatment of myasthenia gravis includes any aspect of myasthenia gravis including, but not limited to skeletal muscle weakness, fatigability, asymmetric ptosis, diplopia, weak neck extensors, drooping of the head, facial snarl when patient attempts to smile due to weakness of facial and bulbar muscles, nasal or dysarthric and low-volume dysphonic speech, dysphagia which can result in choking or regurgitation, and skeletal muscle weakness which can cause difficulties in walking, climbing stairs, or carrying objects. Treatment of systemic scleroderma includes any aspect of systemic scleroderma including, but not limited to, swelling and thickening of the fingers and hand with possible involvement of the face, thickening of the skin, involvement of the trunk and arms proximal to the elbows, skin atrophy with possible loss of hair, sebaceous glands, and sweat glands; loss of pliability of the skin; hidebound skin where the skin is tightly drawn and bound to underlying structures; and limited mobility, especially in the fingers. Treatment of ITP includes any aspect of ITP including, but not limited to presence of petechiae in the lower extremities, mild clinical bleeding consisting of purpura, epistaxis, gingival bleeding, menorrhagia, unpalpable spleen, and in case of several thrombocytopenia, blood blisters in the mouth. Treatment of polyrnyositis includes any aspect of polymyositis including, but not limited to, weakening of primarily skeletal muscle, weakening of proximal muscles, aspiration pneumonia, interstitial lung disease, soft tissue calcification, and Raynaud phenomenon. Treatment of APS (which can also include antibody-mediated thrombosis) includes any aspect of APS including, but not limited to, arterial occlusion, extremity gangrene, stroke, myocardial infarct, other visceral infarct, venous occlusion, peripheral venous occlusion, visceral venous occlusion (e.g., Budd-Chiari syndrome, portal vein occlusion), recurrent fetal loss, thrombocytopenia, Coombs'-positive hemolytic anemia, livedo reticularis, neurological abnormalities (e.g., chorea, transient ischemic attacks), valvular heart disease, and sudden multisystem occlusion.

### Kits

A CD21 binding protein described herein can be provided in a kit, e.g., as a component of a kit. For example, the kit includes (a) an antibody, an antigen fragment or a polynucleotide described herein, and, optionally (b) informational material. The informational material can be descriptive, instructional, marketing or other material that relates to the methods described herein and/or the use of an antibody, an antigen fragment or a polynucleotide described herein for the methods described herein.

The informational material of the kits is not limited in its form. In one embodiment, the informational material can include information about production of the antibody, antigen binding fragment or the polynucleotide encoding the antibody or the antigen binding fragment, their molecular weight, concentration, date of expiration, batch or production site information, and so forth. In one embodiment, the informational material relates to using the antibody, antigen binding fragment or the polynucleotide to treat, prevent, or diagnosis of disorders and conditions, e.g., autoimmune diseases or disorders.

In one embodiment, the informational material can include instructions to administer the antibody, the antigen binding fragment or the polynucleotide in a suitable manner to perform the methods described herein, e.g., in a suitable dose, dosage form, or mode of administration (e.g., a dose, dosage form, or mode of administration described herein). In another embodiment, the informational material can include instructions to administer the antibody, the antigen binding fragment or the polynucleotide to a suitable subject, e.g., a human, e.g., a human having, or at risk for, a disorder or condition described herein, e.g., systemic lupus erythematosis.

The informational material of the kits is not limited in its form. In many cases, the informational material, e.g., instructions, is provided in print but can also be in other formats, such as computer readable material.

The the antibody, antigen binding fragment or the polynucleotide can be provided in any form, e.g., liquid, dried or lyophilized form. It is preferred that the antibody, antigen binding fragment or the polynucleotide be substantially pure and/or sterile. When the antibody, antigen binding fragment or the polynucleotide is provided in a liquid solution, the liquid solution preferably is an aqueous solution, with a sterile aqueous solution being preferred. When the antibody, antigen binding fragment or the polynucleotide is provided as a dried form, reconstitution generally is by the addition of a suitable solvent. The solvent, e.g., sterile water or buffer, can optionally be provided in the kit.

The kit can include one or more containers for the composition containing the antibody, antigen binding fragment or the polynucleotide. In some embodiments, the kit contains separate containers, dividers or compartments for the composition and informational material. For example, the composition can be contained in a bottle, vial, or syringe, and the informational material can be contained association with the container. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of an antibody or antigen binding fragment or a polynyucleotide. For example, the kit includes a plurality of syringes, ampules, foil packets, or blister packs, each containing a single unit dose of an antibody or antigen binding fragment or a polynyucleotide. The containers of the kits can be air tight, waterproof (e.g., impermeable to changes in moisture or evaporation), and/or light-tight.

The kit optionally includes a device suitable for administration of the composition, e.g., a syringe, inhalant, dropper (e.g., eye dropper), swab (e.g., a cotton swab or wooden swab), or any such delivery device. In one embodiment, the device is an implantable device that dispenses metered doses of the binding protein. The disclosure also features a method of providing a kit, e.g., by combining components described herein.

The invention may be as described in any one of the following numbered paragraphs:
1. An antibody or antigen binding fragment that binds to CD21 and comprises:
a. a heavy chain variable region (V_{H}) comprising:
   i. a complementarity determining region 1 (CDR_H1) sequence comprising an amino acid sequence that differs by no more than four amino acids from the amino acid sequence G-G-X₁-X₂-X₃-X₄-X₅-A-I-S (SEQ ID NO: 102), wherein X₁ is T or E; X₂ is F or L; X₃ is S, or Q; X₄ is T, D, or S; X₅ is Y or L;
   ii. a complementarity determining region 2 (CDR_H2) sequence comprising an amino acid sequence that differs by no more than four amino acids from the amino acid sequence RIIPILGIANYAQKFQG (SEQ ID NO: 3); or
   iii. a complementarity determining region 3 (CDR_H3) sequence comprising an amino acid sequence that differs by no more than four amino acids from the amino acid sequence E-X₆-D-X₇-S-G-Y-Y-Q-D (SEQ ID NO: 103), wherein X₆ is D or E; X₇ is S or I; or
b. a light chain variable region (V_{L}) comprising:
   i. a complementarity determining region 1 (CDR_L1) sequence comprising an amino acid sequence that differs by no more than four amino acids from the amino acid sequence R-A-G-Q-X₈-I-X₉-X₁₀-Y-L-A (SEQ ID NO: 104), wherein X₈ is S or K; X₉ is N or S; X₁₀ is S, H, or R;
   ii. a complementarity determining region 2 (CDR_L2) sequence comprising an amino acid sequence that differs by no more than four amino acids from the amino acid sequence D-A-S-X₁₁-R-A-T (SEQ ID NO: 105), wherein X₁₁ is S or T; or
   iii. a complementarity determining region 3 (CDR_L3) sequence comprising an amino acid sequence that differs by no more than four amino acids from the amino acid sequence Q-Q-Y-E-Y-W-Y-X₁₂ (SEQ ID NO: 106), wherein X₁₂ is S or T.
2. The antibody or antigen binding fragment of paragraph 1, wherein the antibody or antigen-binding fragment comprises a nanobody, an scFv, a monoclonal antibody, a humanized antibody, a human antibody, a recombinant antibody, a chimeric antibody, or a Fab fragment.
3. The antibody or antigen binding fragment of paragraph 1 or 2, wherein the CDR_H1 comprises an amino acid sequence that differs by no more than four amino acids from an amino acid sequence selected from the group consisting of: SEQ ID NOs.: 2, 30, 36, 44, 81, 84, 87, 90, 93, 96, and 99.
4. The antibody or antigen binding fragment of any one of paragraphs 1-3, wherein the CDR_H2 comprises an amino acid sequence that differs by no more than four amino acids from SEQ ID NO: 3.
5. The antibody or antigen binding fragment of any one of paragraphs 1-4, wherein the CDR_H3 comprises an amino acid sequence that differs by no more than four amino acids from an amino acid sequence selected from the group consisting of: SEQ ID NOs.: 4, 37, and 51.
6. The antibody or antigen binding fragment of any one of paragraphs 1-5, wherein the CDR_LI comprises an amino acid sequence that differs by no more than four amino acids from an amino acid sequence selected from the group consisting of: SEQ ID NOs.: SEQ ID NOs: 15, 33, 54, 59, 62 and 65.
7. The antibody or antigen binding fragment of any one of paragraphs 1-6, wherein the CDR_L2 comprises an amino acid sequence that differs by no more than four amino acids from an amino acid sequence selected from the group consisting of: SEQ ID NOs.: 16 and 68.
8. The antibody or antigen binding fragment of any one of paragraphs 1-7, wherein the CDR_L3 comprises an amino acid sequence that differs by no more than four amino acids from an amino acid sequence selected from the group consisting of: SEQ ID NOs.: 17 and 55.
9. The antibody or antigen binding fragment of any one of paragraphs 1-8, wherein CDR_H1 comprises an amino acid sequence that differs by no more than four amino acids from SEQ ID NO: 2, CDR_H2 comprises an amino acid sequence that differs by no more than four amino acids from SEQ ID NO: 3, and CDR_H3 comprises an amino acid sequence that differs by no more than four amino acids from SEQ ID NO: 4; or
10. The antibody or antigen binding fragment of any one of paragraphs 1-8, wherein CDR_H1 comprises an amino acid sequence that differs by no more than four amino acids from SEQ ID NO: 30, CDR_H2 comprises an amino acid sequence that differs by no more than four amino acids from SEQ ID NO: 3, and CDR_H3 comprises an amino acid sequence that differs by no more than four amino acids from SEQ ID NO: 4; or
11. The antibody or antigen binding fragment of any one of paragraphs 1-8, wherein CDR_H1 comprises an amino acid sequence that differs by no more than four amino acids from SEQ ID NO: 36, CDR_H2 comprises an amino acid sequence that differs by no more than four amino acids from SEQ ID NO: 3, and CDR_H3 comprises an amino acid sequence that differs by no more than four amino acids from SEQ ID NO: 37; or
12. The antibody or antigen binding fragment of any one of paragraphs 1-8, wherein CDR_H1 comprises an amino acid sequence that differs by no more than four amino acids from SEQ ID NO: 44, CDR_H2 comprises an amino acid sequence that differs by no more than four amino acids from SEQ ID NO: 3, and CDR_H3 comprises an amino acid sequence that differs by no more than four amino acids from SEQ ID NO: 37; or
13. The antibody or antigen binding fragment of any one of paragraphs 1-8, wherein CDR_H1 comprises an amino acid sequence that differs by no more than four amino acids from SEQ ID NO: 2, CDR_H2 comprises an amino acid sequence that differs by no more than four amino acids from SEQ ID NO: 3, and CDR_H3 comprises an amino acid sequence that differs by no more than four amino acids from SEQ ID NO: 37; or
14. The antibody or antigen binding fragment of any one of paragraphs 1-8, wherein CDR_H1 comprises an amino acid sequence that differs by no more than four amino acids from SEQ ID NO: 30, CDR_H2 comprises an amino acid sequence that differs by no more than four amino acids from SEQ ID NO: 3, and CDR_H3 comprises an amino acid sequence that differs by no more than four amino acids from SEQ ID NO: 37; or
15. The antibody or antigen binding fragment of any one of paragraphs 1-8, wherein CDR_H1 comprises an amino acid sequence that differs by no more than four amino acids from SEQ ID NO: 2, CDR_H2 comprises an amino acid sequence that differs by no more than four amino acids from SEQ ID NO: 3, and CDR_H3 comprises an amino acid sequence that differs by no more than four amino acids from SEQ ID NO: 51; or
16. The antibody or antigen binding fragment of any one of paragraphs 1-8, wherein CDR_H1 comprises an amino acid sequence that differs by no more than four amino acids from SEQ ID NO: 44, CDR_H2 comprises an amino acid sequence that differs by no more than four amino acids from SEQ ID NO: 3, and CDR_H3 comprises an amino acid sequence that differs by no more than four amino acids from SEQ ID NO: 51; or
17. The antibody or antigen binding fragment of any one of paragraphs 1-8, wherein CDR_H1 comprises an amino acid sequence that differs by no more than four amino acids from SEQ ID NO: 81, CDR_H2 comprises an amino acid sequence that differs by no more than four amino acids from SEQ ID NO: 3, and CDR_H3 comprises an amino acid sequence that differs by no more than four amino acids from SEQ ID NO: 51; or
18. The antibody or antigen binding fragment of any one of paragraphs 1-8, wherein CDR_H1 comprises an amino acid sequence that differs by no more than four amino acids from SEQ ID NO: 84, CDR_H2 comprises an amino acid sequence that differs by no more than four amino acids from SEQ ID NO: 3, and CDR_H3 comprises an amino acid sequence that differs by no more than four amino acids from SEQ ID NO: 51; or
19. The antibody or antigen binding fragment of any one of paragraphs 1-8, wherein CDR_H1 comprises an amino acid sequence that differs by no more than four amino acids from SEQ ID NO: 87, CDR_H2 comprises an amino acid sequence that differs by no more than four amino acids from SEQ ID NO: 3, and CDR_H3 comprises an amino acid sequence that differs by no more than four amino acids from SEQ ID NO: 51; or
20. The antibody or antigen binding fragment of any one of paragraphs 1-8, wherein CDR_H1 comprises an amino acid sequence that differs by no more than four amino acids from SEQ ID NO: 90, CDR_H2 comprises an amino acid sequence that differs by no more than four amino acids from SEQ ID NO: 3, and CDR_H3 comprises an amino acid sequence that differs by no more than four amino acids from SEQ ID NO: 51; or
21. The antibody or antigen binding fragment of any one of paragraphs 1-8, wherein CDR_H1 comprises an amino acid sequence that differs by no more than four amino acids from SEQ ID NO: 93, CDR_H2 comprises an amino acid sequence that differs by no more than four amino acids from SEQ ID NO: 3, and CDR_H3 comprises an amino acid sequence that differs by no more than four amino acids from SEQ ID NO: 51; or
22. The antibody or antigen binding fragment of any one of paragraphs 1-8, wherein CDR_H1 comprises an amino acid sequence that differs by no more than four amino acids from SEQ ID NO: 96, CDR_H2 comprises an amino acid sequence that differs by no more than four amino acids from SEQ ID NO: 3, and CDR_H3 comprises an amino acid sequence that differs by no more than four amino acids from SEQ ID NO: 51; or
23. The antibody or antigen binding fragment of any one of paragraphs 1-8, wherein CDR_H1 comprises an amino acid sequence that differs by no more than four amino acids from SEQ ID NO: 99, CDR_H2 comprises an amino acid sequence that differs by no more than four amino acids from SEQ ID NO: 3, and CDR_H3 comprises an amino acid sequence that differs by no more than four amino acids from SEQ ID NO: 51.
24. The antibody or antigen binding fragment of any one of paragraphs 1-23, wherein CDR_LI comprises an amino acid sequence that differs by no more than four amino acids from SEQ ID NO: 15, CDR_L2 comprises an amino acid sequence that differs by no more than four amino acids from SEQ ID NO: 16, and CDR_L3 comprises an amino acid sequence that differs by no more than four amino acids from SEQ ID NO: 17.
25. The antibody or antigen binding fragment of any one of paragraphs 1-23, wherein CDR_L1 comprises an amino acid sequence that differs by no more than four amino acids from SEQ ID NO: 33, CDR_L2 comprises an amino acid sequence that differs by no more than four amino acids from SEQ ID NO: 16, and CDR_L3 comprises an amino acid sequence that differs by no more than four amino acids from SEQ ID NO: 17.
26. The antibody or antigen binding fragment of any one of paragraphs 23, wherein CDR_L1 comprises an amino acid sequence that differs by no more than four amino acids from SEQ ID NO: 54, CDR_L2 comprises an amino acid sequence that differs by no more than four amino acids from SEQ ID NO: 16, and CDR_L3 comprises an amino acid sequence that differs by no more than four amino acids from SEQ ID NO: 55.
27. The antibody or antigen binding fragment of any one of paragraphs 23, wherein CDR_L1 comprises an amino acid sequence that differs by no more than four amino acids from SEQ ID NO: 59, CDR_L2 comprises an amino acid sequence that differs by no more than four amino acids from SEQ ID NO: 16, and CDR_L3 comprises an amino acid sequence that differs by no more than four amino acids from SEQ ID NO: 55.
28. The antibody or antigen binding fragment of any one of paragraphs 23, wherein CDR_L1 comprises an amino acid sequence that differs by no more than four amino acids from SEQ ID NO: 62, CDR_L2 comprises an amino acid sequence that differs by no more than four amino acids from SEQ ID NO: 16, and CDR_L3 comprises an amino acid sequence that differs by no more than four amino acids from SEQ ID NO: 55.
29. The antibody or antigen binding fragment of any one of paragraphs 23, wherein CDR_L1 comprises an amino acid sequence that differs by no more than four amino acids from SEQ ID NO: 65, CDR_L2 comprises an amino acid sequence that differs by no more than four amino acids from SEQ ID NO: 16, and CDR_L3 comprises an amino acid sequence that differs by no more than four amino acids from SEQ ID NO: 55.
30. The antibody or antigen binding fragment of any one of paragraphs 23, wherein CDR_L1 comprises an amino acid sequence that differs by no more than four amino acids from SEQ ID NO: 54, CDR_L2 comprises an amino acid sequence that differs by no more than four amino acids from SEQ ID NO: 68, and CDR_L3 comprises an amino acid sequence that differs by no more than four amino acids from SEQ ID NO: 55.
31. The antibody or antigen binding fragment of any one of paragraphs 23, wherein CDR_L1 comprises an amino acid sequence that differs by no more than four amino acids from SEQ ID NO: 59, CDR_L2 comprises an amino acid sequence that differs by no more than four amino acids from SEQ ID NO: 68, and CDR_L3 comprises an amino acid sequence that differs by no more than four amino acids from SEQ ID NO: 55.
32. The antibody or antigen binding fragment of any one of paragraphs 23, wherein CDR_L1 comprises an amino acid sequence that differs by no more than four amino acids from SEQ ID NO: 62, CDR_L2 comprises an amino acid sequence that differs by no more than four amino acids from SEQ ID NO: 68, and CDR_L3 comprises an amino acid sequence that differs by no more than four amino acids from SEQ ID NO: 55.
33. The antibody or antigen binding fragment of any one of paragraphs 23, wherein CDR_L1 comprises an amino acid sequence that differs by no more than four amino acids from SEQ ID NO: 65, CDR_L2 comprises an amino acid sequence that differs by no more than four amino acids from SEQ ID NO: 68, and CDR_L3 comprises an amino acid sequence that differs by no more than four amino acids from SEQ ID NO: 55.
34. The antibody or antigen binding fragment of any one of paragraphs 1-33, wherein the heavy chain variable region further comprises a Framework region 1 (FW_H1) sequence that differs by no more than four, preferably no more than three, more preferably no more than two, most preferably no more than one amino acid from SEQ ID NO: 10.
35. The antibody or antigen binding fragment of any one of paragraphs 1-34, wherein the heavy chain variable region further comprises a Framework region 2 (FW_H2) sequence that differs by no more than four, preferably no more than three, more preferably no more than two, most preferably no more than one amino acid from SEQ ID NO: 11.
36. The antibody or antigen binding fragment of any one of paragraphs 1-35, wherein the heavy chain variable region further comprises a Framework region 3 (FW_H3) sequence that differs by no more than four, preferably no more than three, more preferably no more than two, most preferably no more than one amino acid from SEQ ID NO: 12.
37. The antibody or antigen binding fragment of any one of paragraphs 1-36, wherein the heavy chain variable region further comprises a Framework region 4 (FW_H4) sequence that differs by no more than four, preferably no more than three, more preferably no more than two, most preferably no more than one amino acid from SEQ ID NO: 13.
38. The antibody or antigen binding fragment of any one of paragraphs 1-37, wherein the heavy chain variable region comprises an amino acid sequence having at least 85%, identity to an amino acid sequence selected from the group consisting of SEQ ID NOs: 9, 31, 38, 45, 47, 49, 52, 77, 82, 85, 88, 91, 94, 97 and 100.
39. The antibody or antigen binding fragment of any one of paragraphs 1-38, wherein the antibody or antigen binding fragment comprises a heavy chain constant region 1 (CH1) comprising an amino acid sequence having at least 85% identity to SEQ ID NO: 5.
40. The antibody or antigen binding fragment of any one of paragraphs 1-39, wherein the antibody or antigen binding fragment comprises a heavy chain constant region 2 (CH2) comprising an amino acid sequence having at least 85% identity to SEQ ID NO: 7.
41. The antibody or antigen binding fragment of any one of paragraphs 1-40, wherein the antibody or antigen binding fragment comprises a heavy chain constant region 1 (CH3) comprising an amino acid sequence having at least 85% identity to SEQ ID NO: 8 or 40.
42. The antibody or antigen binding fragment of any one of paragraphs 1-41, wherein the antibody or antigen binding fragment comprises a heavy chain hinge region (CH1_CH2_hinge) comprising an amino acid sequence having at least 85% identity to SEQ ID NO: 6.
43. The antibody or antigen binding fragment of any one of paragraphs 1-42, wherein the antibody or antigen-binding fragment comprises a heavy chain amino acid sequence having at least 85% identity to an amino acid sequence selected from the group consisting of SEQ ID NOs: 1, 29, 35, 39, 41, 42, 43, 46, 48, 50, 76, 80, 83, 86, 89, 92, 95, 98 and 101.
44. The antibody or antigen binding fragment of any one of paragraphs 1-43, wherein the light chain variable region further comprises a Framework region 1 (FW_L1) sequence that differs by no more than four amino acids from SEQ ID NO: 20, 26 or 57.
45. The antibody or antigen binding fragment of any one of paragraphs 1-44, wherein the light chain variable region further comprises a Framework region 2 (FW_L2) sequence that differs by no more than four amino acids from SEQ ID NO: 21 or 27.
46. The antibody or antigen binding fragment of any one of paragraphs 1-45, wherein the light chain variable region further comprises a Framework region 3 (FW_L3) sequence that differs by no more than four amino acids from SEQ ID NO: 23 or 29.
47. The antibody or antigen binding fragment of any one of paragraphs 1-46, wherein the light chain variable region further comprises a Framework region 4 (FW_L4) sequence that differs by no more than four amino acids from SEQ ID NO: 23.
48. The antibody or antigen binding fragment of any one of paragraphs 1-47, wherein the light chain variable region comprises an amino acid sequence having at least 85% identity to an amino acid sequence selected from the group consisting of SEQ ID NOs: 19, 25, 34, 56, 60, 63, 66, 69, 71, 73, 75 and 79.
49. The antibody or antigen binding fragment of any one of paragraphs 1-48, wherein the antibody or antigen binding fragment comprises a light chain constant region (C_{L}) comprising an amino acid sequence having at least 85% identity to SEQ ID NO: 18.
50. The antibody or antigen binding fragment of any one of paragraphs 1-49, wherein the antibody or antigen binding fragment a light chain amino acid sequence having at least 85% identity to an amino acid sequence selected from the group consisting of SEQ ID NOs: 24, 32, 53, 58, 61, 64, 67, 70, 72, 74 and 78.
51. The antibody or antigen binding fragment of any one of paragraphs 1-50, wherein the antibody or antigen binding fragment is a CD21 inhibitor or a CD21 neutralizing antibody.
52. The antibody or antigen binding fragment of any one of paragraphs 1-51, wherein the antibody or antigen binding fragment reduces CD21 activity and/or B cell activation by at least 20% as compared to CD21 activity and/or B cell activation in the absence of the antibody or antigen-binding fragment.
53. The antibody or antigen binding fragment of any one of paragraphs 1-52, wherein the antibody or antigen binding fragment disrupts binding of CD21 to its cognate ligand C3d.
54. A composition comprising an antibody or antigen binding fragment of any one of paragraphs 1-53.
55. The composition of paragraph 54, wherein the composition comprises a pharmaceutically acceptable excipient or carrier.
56. A cell comprising an antibody or antigen binding fragment of any one of paragraphs 1-53.
57. A polynucleotide comprising a nucleotide sequence encoding an antibody or antigen binding fragment of any one of paragraphs 1-53.
58. The polynucleotide of paragraph 57, wherein the polynucleotide is comprised in a vector.
59. A composition comprising a polynucleotide paragraph 57 or 58.
60. A cell comprising a polynucleotide of paragraph 57 or 58.
61. A kit comprising:
   a. an antibody or antigen binding fragment of any one of paragraphs 1-53;
   b. a polynucleotide of paragraph 57 or 58;
   c. a composition of paragraph 54, 55, 58 or 59; or
   d. a cell of paragraph 56 or 60.
62. The kit of paragraph 61, further comprising instructions for use thereof.
63. A method for reducing CD21 activity or CD21/C3d mediated B cell activation, the method comprising: administering an antibody or antigen-binding fragment of any one of paragraphs 1-53 or a polynucleotide of paragraph 57 or 58 to a B cell.
64. The method of paragraph 63, wherein said administering to the B cell is in vitro.
65. The method of paragraph 63, wherein said administering to the B cell is in vivo.
66. The method of paragraph 65, wherein said administering to the cell is in a subject diagnosed with or in need of treatment for an autoimmune disease or disorder.
67. A method for reducing at least one symptom of an autoimmune disease or disorder, the method comprising administering a therapeutically effective amount of an antibody or antigen-binding fragment of any one of paragraphs 1-53 or a polynucleotide of paragraph 57 or 58 to a subject in need thereof.
68. The method of paragraph 67, wherein the autoimmune disease or disorder comprises systemic lupus erythematosus (SLE).
69. The method of paragraph 68, wherein the at least one symptom of SLE comprises lupus nephritis, malar rash, discoid rash, butterfly rash, photosensitivity, oral ulcers, arthritis, serositis, pleuritis, pericarditis, proteinuria, seizures, psychosis, hemolytic anemia, leukopenia, lymphopenia, or thrombocytopenia.
70. The method of paragraph 68 or 69, wherein the at least one symptom of SLE comprises a clinical measure of SLE selected from the group consisting of: positive lupus erythematosus cell preparation, anti-DNA antibody to native DNA ratio, and anti-SM nuclear antigen antibodies.
71. The method of any one of paragraphs 65-70 further comprising a step of diagnosing the subject with the autoimmune disease or disorder.
72. The method of any one of paragraphs 65-71, wherein the subject is a mammal.
73. The method of any one of paragraphs 65-71, wherein the subject is human.
74. A composition of any one of paragraphs 1-60 for use in the treatment of an autoimmune disease or disorder, or at least one symptom thereof in a subject.

### EXAMPLES

Provided herein, in part, is a fully human IgG1 (effector-less 3A mutant) antibody (Ab658) against CD21 protein that blocks binding of C3d protein to CD21. The Ab658 anti-CD21 antibody is derived from a phage display scFv antibody clone, SC38-36C01, following multiple rounds of affinity maturation and sequence optimization to improve biophysical properties and reduce sequence liabilities. Also provided herein is the IgG1 version of the initial phage hit 36C01, called Ab15, which was characterized for its binding and neutralization activity. The affinity and potency of the Ab15 antibody was inferior to Ab658 so no additional affinity optimization was performed.

The affinity maturation and optimization process that resulted in the antibody Ab658 was guided by several of co-crystal structures of clones related to Ab15 with human CD21 (huCD21). During the affinity maturation process, human CD21 affinity was improved from approximately 16 nM to 0.04 nM. Final optimization focused on removing several sequence liabilities and minimizing predicted T-cell epitope content. The final lead antibody Ab658 (same as Ab652, except having C-terminal Lys on HC) has high affinity to human and cynomolgus (cyno) monkey CD21 and good biophysical properties. It neutralizes CD21-mediated functions by blocking C3d binding to CD21.

### Example 1: Generation of Anti-Cd21 Antibodies and Analysis of Early Hits

### Selection of Anti-CD21 Antibodies by Phage Display

Anti-CD21 single-chain Fv (scFv) hits were selected from the human phage display antibody library WyNH5 by panning on the extracellular domain (ECD) of human CD21 and cyno CD21 fused to human Fc, as follows.

Human and cyno CD21-hFc proteins were biotinylated with sulfo-NHS-LC-Biotin (Pierce) according to the manufacturer's protocol. Biotinylated CD21-Fc proteins were first captured on streptavidin-coated magnetic Dynabeads™ M-280 (Invitrogen). The beads with immobilized CD21-Fc were then used to select binders from WyNH5 library using standard procedures. Four rounds of selection were performed with decreasing concentrations of the target (human or cyno CD21-hFc), as follows: 200 nM huCD21-hFc (1^{st} round), 50 nM cyCD21-hFc (2^{nd} round), 10 nM huCD21-hFc (3^{rd} round), and 2 nM cyCD21-hFc (4^{th} round). To obtain antibodies specific to CD21, which do not substantially bind the human Fc portion, all selections were performed in the presence of 500 nM unrelated human IgG1. In each selection round, CD21-specific binders were eluted with 100 mM triethylamine.

ScFv can be expressed either on the surface of a phage particle or in solution in the bacterial periplasmic space, depending upon the growth conditions used. To induce release of scFv into the periplasm, 96-deep well plates containing 2×YT media with 0.1% glucose and 100 μg/ml ampicillin were inoculated from 2×YT agar plates (one clone per well) and grown at 37° C. (850rpm) for ˜4 hours. Cultures were induced with IPTG at a final concentration of 0.02 mM and grown overnight at 30° C. (850 rpm). The contents of the bacterial periplasm (peripreps) were released by osmotic shock. Briefly, plates were centrifuged and pellets were resuspended in 150 μl TES periplasmic buffer (50 mM Tris, 1 mM EDTA, 20% Sucrose, pH7.4), followed by the addition of 150 μl 1:5 TES: water and incubated on ice for 30 minutes. Plates were centrifuged for 20 minutes at 4000 rpm and the scFv-containing supernatants were harvested.

For binding assay with scFv peripreps, high-binding 96-well ELISA plates (Costar 3590) were coated with 0.1 ug per well (100 ul of 1 ug/ml) of human or cyno CD21-Fc in PBS buffer overnight at 4° C. After discarding coating solution, plates were blocked at room temperature for 2 hrs with PBS+3% BSA. After discarding blocking solution, 100 ul of peripreps was added. After 1 hr incubation at room temperature with slow shaking, plates were washed 5 times with 300 ul/well of washing buffer (Perkin Elmer 1244-114) and incubated for 1 hr with 100 ul of secondary HRP-labeled anti-His tag Ab. After washing 5× with 300 microliters per well of washing buffer, plates were developed with TMB substrate solution for 10 min, then 0.18M H₂SO₄ was added to stop the reaction. Absorbance at 450 nM was measured and data were plotted and analyzed with Microsoft Excel. From this screen, 78 scFv hits were identified as binding to human or cyno CD21 by periprep ELISA, and subsequently converted to IgGs using standard molecular biology methods.

### Functional Properties of Anti-CD21 Antibodies

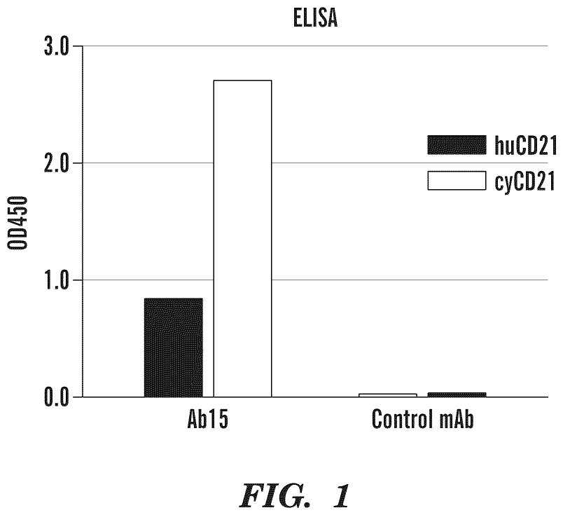

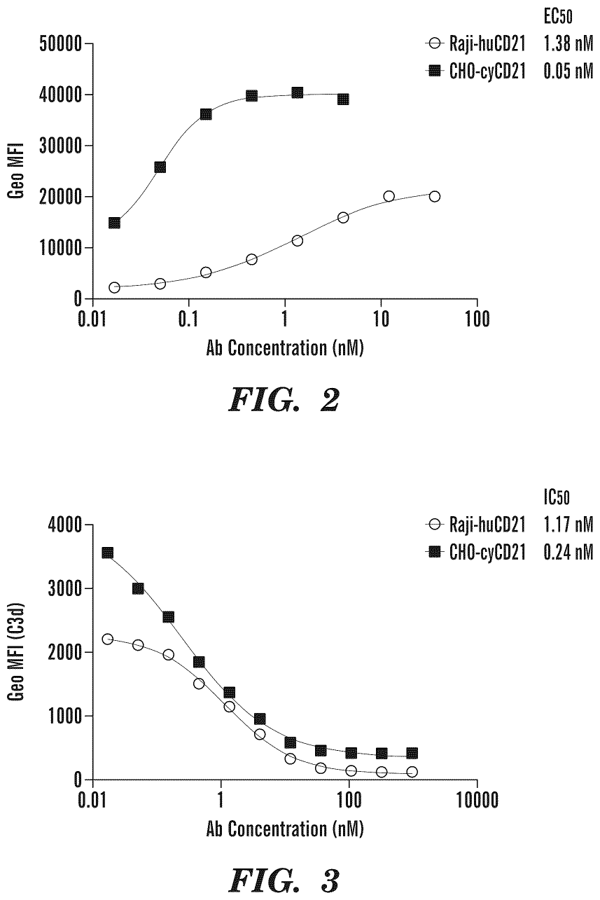

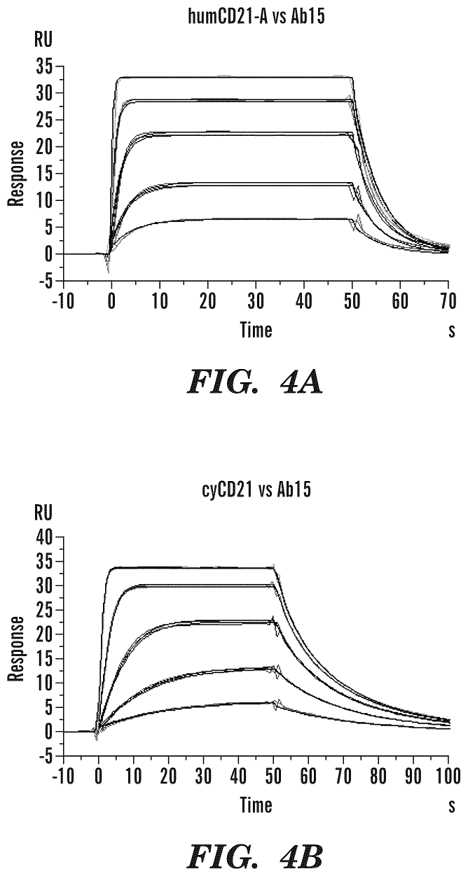

After reformatting from scFv to IgG1, antibody clone 36C01 became known as Ab15. Binding activity of Ab15 to human and cyno CD21 was measured using ELISA. The results are shown in FIG. 1. Ab15 also showed binding to Raji cells (i.e., a human B-cell line) that express CD21 endogenously, and to CHO cells over-expressing cyno CD21, as shown in FIG. 2. Furthermore, Ab15 can inhibit binding of human C3d protein (a CD21 ligand) to Raji cells and CHO-cyno CD21 cells as shown in FIG. 3. Measurements using SPR (Biacore) indicate that Ab15 binds to human and cyno CD21 with an affinity of 15.59 nM and 5.77 nM, respectively, as illustrated in FIG. 4.

### Binding of Ab15 to Human and Cyno CD21 Protein by ELISA.

Purified human and cynomolgus CD21 ECD proteins were coated on Nunc-Maxisorb™ 96-well ELISA plates at 2 ug/ml in 100 μl in PBS-CMF (calcium and magnesium free) at 4° C. Plates were washed 3 times with PBS+0.05% Tween-20 and blocked with PBS+3% milk for 1 hr at RT with shaking. Blocking solution was removed and 1 ug/ml Ab 15 was added and incubated for 1 hr at RT with shaking. Plates were washed as before and a secondary antibody was added (goat anti-human IgG Fc-HRP from Southern Biotech, 1:5000), followed by 1 hr incubation at RT. Plates were washed as before, signal was developed using TMB substrate and the reaction was stopped with 0.18M H₂SO₄. Absorbance was read at 450 nm on an Envision plate reader (Perkin Elmer). The result is shown in FIG. 1.

### Binding of Ab15 to Cells Expressing Human and Cyno CD21 Measured by Flow Cytometry.

Ab15 binding to cell-surface human and cyno CD21 was determined using human Raji B cells (ATCC, Manassas, VA) and CHO cells over expressing cyno CD21. Cells expressing cyno CD21 were generated by transfecting CHO cells with a mammalian expression construct encoding full-length cyno CD21. Stable cell clones were selected on hygromycin-B, and their expression of cyno CD21 confirmed by flow cytometry.

For the Ab15 binding experiment, cells were dissociated with cell dissociation buffer (Sigma) and blocked on ice with PBS+3% bovine serum (BS)+human Fc blocker. Cells were stained on ice for 1 hr with 3-fold serially diluted Ab15 (starting from 50 nM), then washed with cold PBS. Secondary antibody (anti-human IgG-PE, 1:500 dilution, Invitrogen, cat. #H10104) was added on ice for 1 hr. Cells were washed with and resuspended in PBS, and analyzed for Ab15 binding on a BD Fortessa instrument. The result is shown in FIG. 2.

### Neutralization of C3d Binding to Cells Expressing Human and Cyno CD21.

To measure neutralization of C3d binding to cell-surface CD21 by flow cytometry, human Raji cells and CHO-cyno CD21 cells were used. Cells were dissociated with cell dissociation buffer (Sigma) and blocked on ice with PBS+3% bovine serum albumin (BSA)+human Fc blocker. In a 96-well plate, 5×10+cells were incubated for 0.5 hr on ice with 3-fold serially diluted Ab15 (starting from 100 nM), then with 4.5 ug/ml biotinylated human C3d. SA-BV451 conjugate (BD Bioscience, cat. #563529) was then added at 0.5 ug/ml for 1 hr on ice. Cells were washed again with cold PBS, resuspended in PBS and analyzed for human C3d binding on a BD Fortessa™. The result is shown in FIG. 3. The Ab15's IC50 of neutralization was measured as 1.17 nM and 0.24 nM on Raji and CHO-cynoCD21 cells, respectively.

### Determination of Ab15 Affinity to Human and Cyno CD21 Protein by Surface Plasmon Resonance.

To determine the affinity of Ab15 to human and cyno CD21 protein, Biacore was used.

### Affinity Maturation and Optimization of Ab15.

In order to optimize Ab15, its 6 CDRs were first grafted into the closest preferred germline frameworks (IGHV1-69 for VH, IGKV3-15 for VL), to generate antibody Ab89. Biacore analysis indicated Ab89 affinity of 71.12 nM and 21.45 nM to human and cyno CD21, respectively. Ab89 also showed binding to human Raji B cells and was able to inhibit human C3d binding to Raji B cells, albeit with slightly lower potency than Ab15 (Table 3).

In order to improve affinity and potency of Ab89, two approaches were applied to generate focused phage display libraries of Ab89 by splice overlap extension PCR. The first approach was soft randomization using mutagenic primers containing NNK codon (where N=A/C/G/T, K=G/T) that aimed to mutate H-CDR3 and L-CDR3. Five scFv libraries with a total combined diversity of 3.2×10⁶ were generated and used for selection on human and cyno CD21. All 5 libraries were combined for selection. In the 1st round selection, 0.25 nM biotinylated huCD21-hFc was used in solution to bind phage for 1 hr at RT followed by streptavidin magnetic bead capture. To obtain CD21-specific scFvs with a slow off-rate, washed beads were incubated with 250 nM non-biotinylated huCD21-hFc overnight. Then the beads were washed 10 times with PBST and 10 times with PBS to remove non-specific binding phages. The specific binding phages were eluted with 100 mM triethylamine and rescued for next round of selection. In the 2nd round, 1 nM biotinylated cyCD21-hFc was incubated with the output phage from the 1st round in solution for 1 hr followed by streptavidin magnetic bead capture. In the 3rd round, 0.1 nM huCD21-hFc was used for selection. In the 4th round, 0.25 nM cyCD21-hFc was used. To obtain antibodies specific to CD21, each selection was performed in the presence of a 1000-fold excess of unrelated human IgG1.

A second affinity maturation approach was taken based on the obtained crystal structure of Fab of Ab25 (related to Ab15; described above) complexed to human CD21 D1-D4 protein. Primers containing mutations aimed to mutate H-CDR1, H-CDR2, H-CDR3, L-CDR1, L-CDR2 and L-CDR3 were designed. Nine scFv libraries with a total combined diversity of 8.6×10⁹ were displayed on phage and selected for human and cyno CD21 binding. After rescuing, the 9 libraries were combined and underwent 4 rounds of selection as described above for the soft randomization approach.

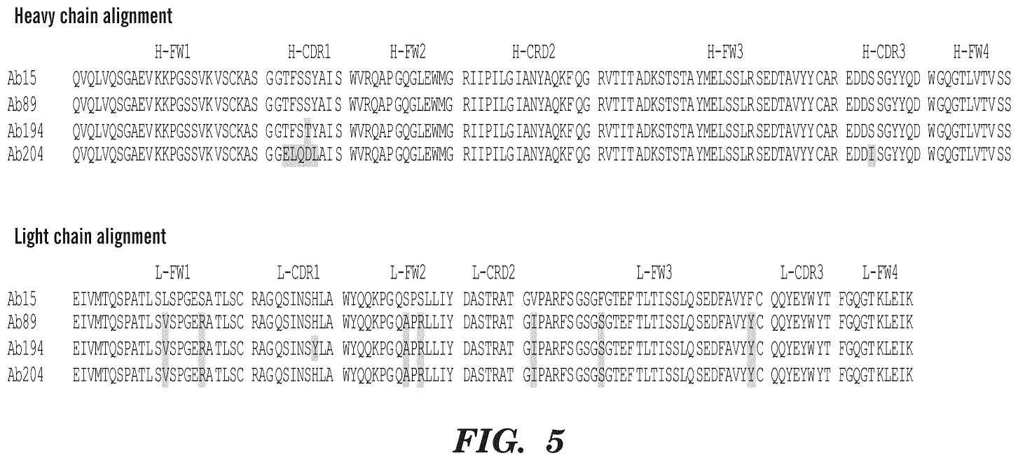

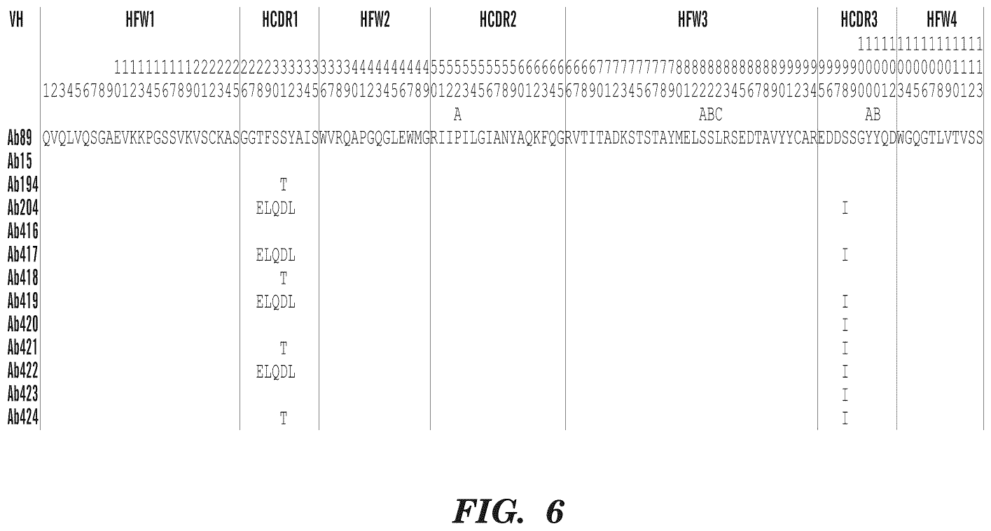

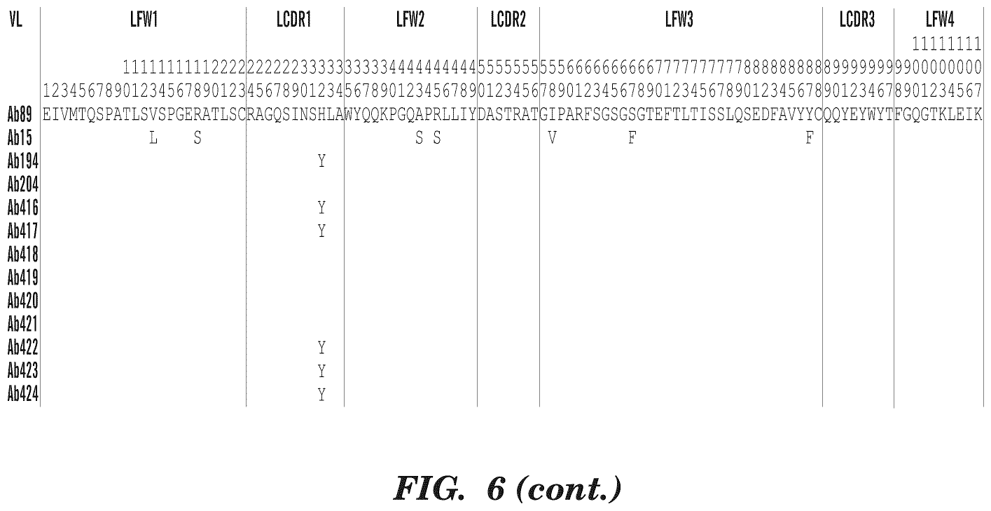

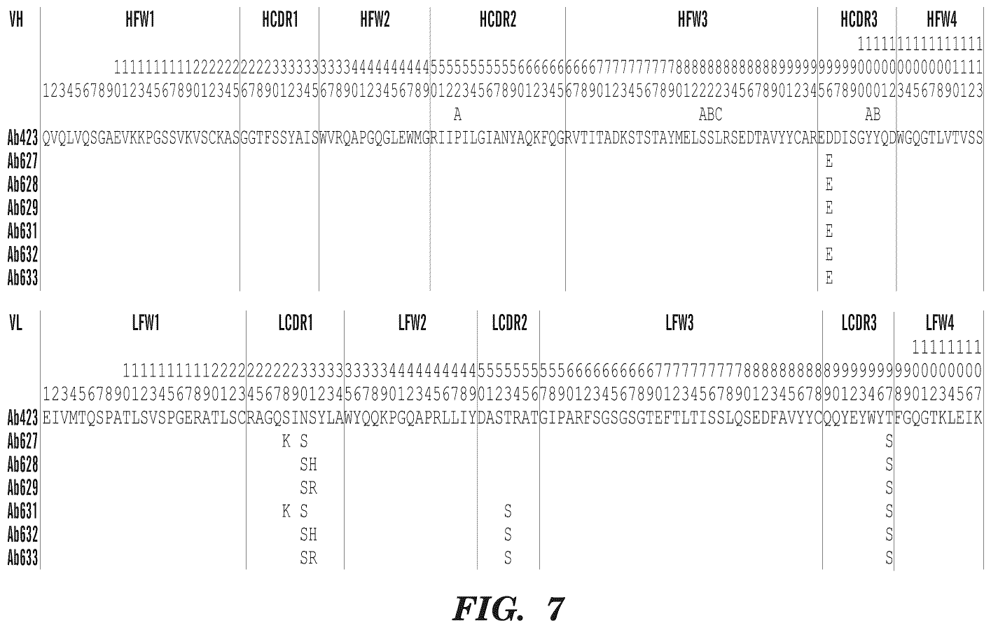

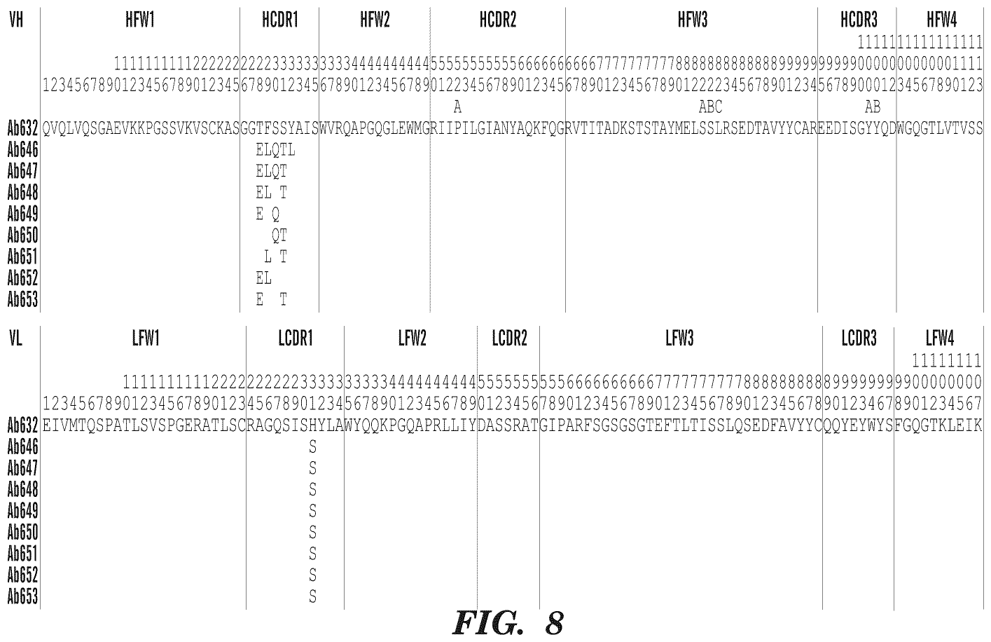

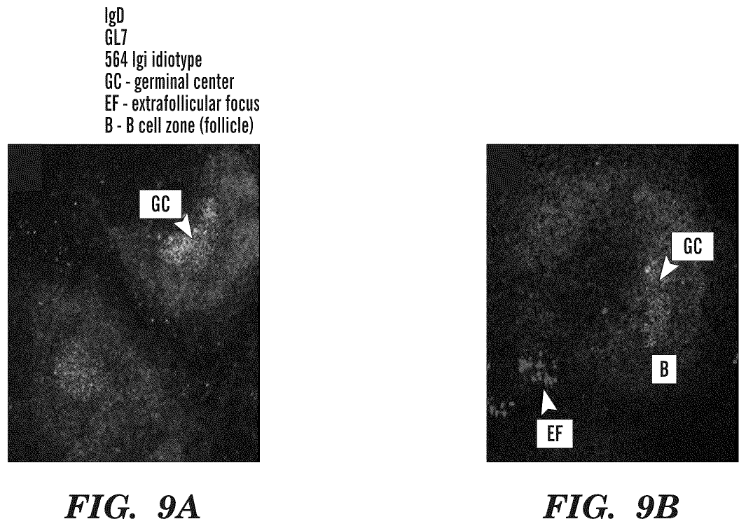

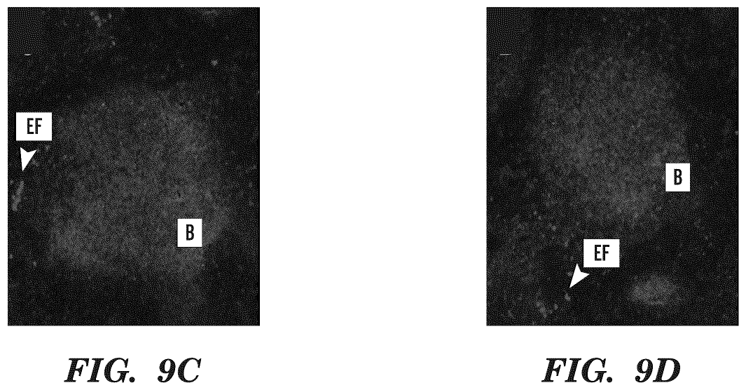

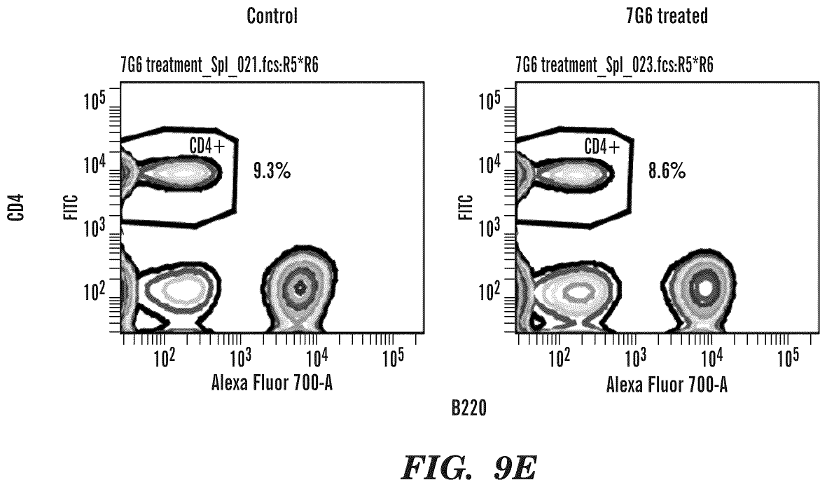

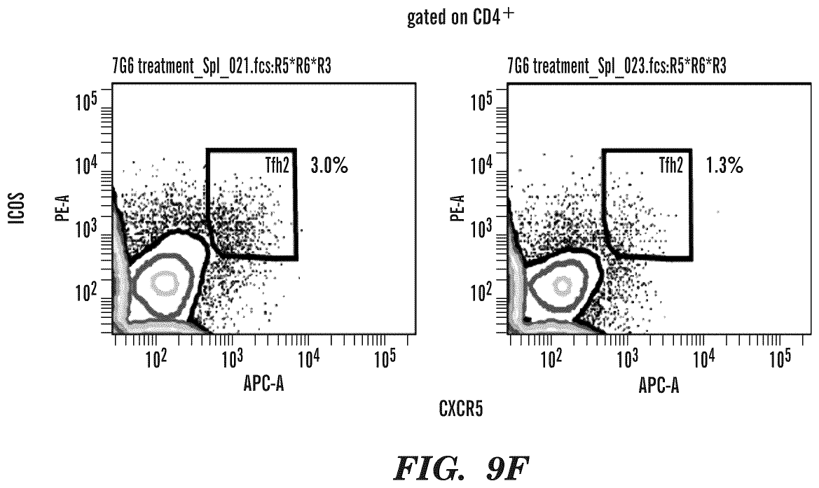

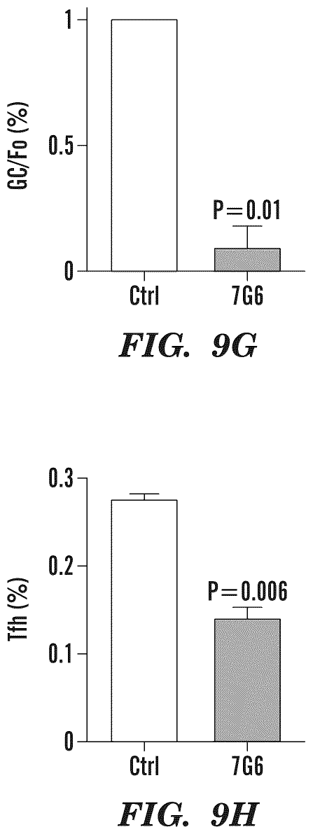

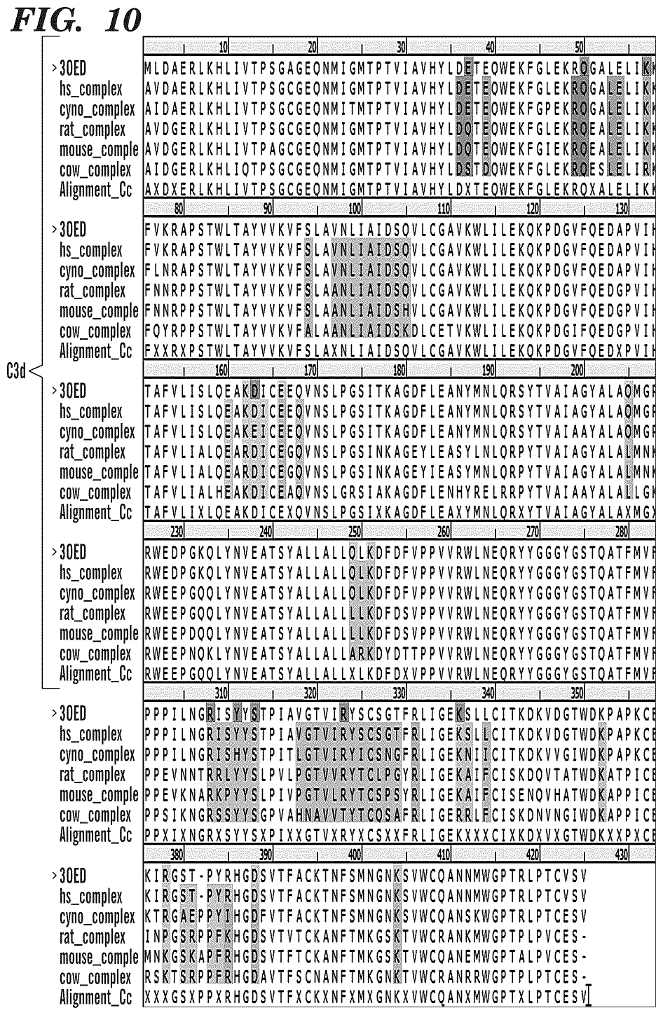

From the two approaches, a total of 2208 clones from the output phage pools were screened in scFv periprep format in a homogenous time resolved fluorescence (HTRF) assay. The HTRF assay was performed as follows. 0.7 nM terbium (Tb)-labeled parental Ab15 was incubated with 100 nM biotinylated human or cyno CD21-hFc protein and 50 nM streptavidin-d2, in the presence of anti-CD21 phage scFv for 1 hr. After incubation, fluorescence at 665 nm and 620 nm was measured on an Envision multi-label plate reader. The HTRF Ratio was calculated as fluorescence at 665 nm/620 nm×10,000. Maximal signal was defined as the HTRF ratio of Tb-labeled Ab15 with d2-labeled CD21 in the absence of antibody, and the minimal signal was defined as the HTRF ratio with d2-labeled CD21 only. The parental Ab15 scFv was used as a control. If the tested Ab gave lower signal than parental Ab15, it indicated that this antibody had higher affinity than parental Ab15. Fifty-three unique clones that showed stronger HTRF competition than the parental Ab15 were identified and reformatted into full-length human IgG1. Two antibodies, Ab194 and Ab204, were prioritized further. The sequence alignments of Ab194 and Ab204 VH and VL domains with those of Ab15 and Ab89 are shown in FIG. 5.

By Biacore, both Ab194 and Ab204 showed binding to human CD21 with relative K_{D} value of 0.81 nM, exhibiting ˜81-fold improvement over the affinity of Ab89 (71 nM). They also showed binding to cyno CD21 with K_{D} value of 9.16 nM and 0.06 nM, respectively, exhibiting 2- and 357-fold improvement, respectively, over the K_{D} of Ab89 on cyCD21 (21.4 nM). Ab194 and Ab204 also showed dramatic improvement in the ability to inhibit human C3d binding to human Raji B cells or primary B cells (Table 3). Additional techniques were used to further characterize physicochemical properties of anti-CD21 antibodies, such as polyspecificity and propensity to self-associate (Table 3). These methods are DNA and Insulin polyreactivity ELISA and AC-SINS assay, described in detail below.

### DNA and Insulin Polyreactivity ELISA Assay

384-well ELISA plates (Nunc Maxisorp) were coated overnight at 4° C. with DNA (10 mg/ml) (Sigma-Aldrich, D1626) and insulin (5 mg/ml) (Sigma-Aldrich, I9278-5 mL) in PBS pH 7.5. The ELISA was carried out on a PerkinElmer Janus Automated Workstation liquid handling robot. Wells were washed with water, blocked with 50 ml of Polyreactivity ELISA Buffer (PEB; PBS containing 0.05% Tween-20, 1 mM EDTA) for 1 hour at room temperature, and rinsed three times with water. Serially diluted mAbs in 25 ml were added in quadruplicate to the wells and incubated for 1 h at room temperature. Plates were washed three times with water, and 25 ml of 10 ng/ml goat anti-human IgG (Fc specific) conjugated to horseradish peroxidase (Jackson ImmunoResearch, 109-035-008) were added to each well. Plates were incubated for 1 h at room temperature, washed three times with 80 ml of water, and 25 ml of TMB substrate (Sigma-Aldrich, T-0440) added to each well. Reactions were stopped after approximately 7 minutes by adding 25 ml of 0.18 M ortho-phosphoric acid to each well, and absorbance was read at 450 nm. DNA-and insulin-binding scores were calculated as the ratio of the ELISA signal of the antibody at 10 mg/ml to the signal of a well containing buffer instead of the primary antibody.

### AC-SINS Assay

The affinity-capture self-interaction nanoparticle spectroscopy (AC-SINS) method was standardized in a 384-well format on a Perkin-Elmer Janus liquid handling robot. 20 nm gold nanoparticles (Ted Pella, Inc., #15705) were coated with a mixture of 80% goat anti-human Fc (Jackson ImmunoResearch Laboratories, cat #109-005-098) and 20% non-specific goat polyclonal antibodies (Jackson ImmunoResearch Laboratories, cat #005-000-003) that were buffer-exchanged into 20 mM sodium acetate pH 4.3 and diluted to 0.4 mg/ml. After one-hour incubation at room temperature, sites unoccupied on the gold nanoparticles were blocked with thiolated polyethylene glycol (2 kD). The coated nanoparticles were then concentrated 10-fold using a syringe filter and 10 ml were added to 100 ml of mAb at 0.05 mg/ml in PBS pH 7.2. The coated nanoparticles were incubated with the antibody of interest for 2 hrs in a 96-well polypropylene plate and then transferred to a 384-well polystyrene plate and read on a Tecan M1000 spectrophotometer. The absorbance was read from 450-650 nm in 2 nm increments, and a Microsoft Excel macro was used to identify the max absorbance, smooth the data, and fit the data using a second-order polynomial. The smoothed max absorbance of the average blank (PBS alone) was subtracted from the smoothed max absorbance of the antibody sample to determine the antibody AC-SINS score.

Additional in silico analysis of anti-CD21 antibodies included calculation of immunogenicity (Epivax) score, as well as number of predicted T-cell epitopes (Table 3).

TABLE 3
Characterization of early anti-CD21 antibodies

| | In silico | | | | | | |
|---|---|---|---|---|---|---|---|
| | immuno- | | | | | | |
| | genicity | | | | | C3d | |
| | assessment | | | | Raji | neutralization | |
| | | # of non- | | | B cell | IC50 (nM) | |
| | | germline | Affinity, | | binding, | Raji | |
| Ab | Epivax | CDR | KD (nM) | | EC50 | B | Primary |
| name | score | epitopes | huCD21 | cyCD21 | (nM) | cells | B cells |
| Ab15 | −33.8 | 4 | 15.59 | 5.77 | 1.38 | 1.17 | Nd |
| Ab89 | −38.89 | 4 | 71.12 | 21.45 | 6.37 | 2.133 | 1.224 |
| Ab194 | −38.03 | 5 | 0.81 | 9.16 | nd | 0.009 | 0.006 |
| Ab204 | −40.98 | 5 | 0.81 | 0.06 | nd | 0.005 | 0.007 |

nd: not determined

Ab194 and Ab204 underwent additional heavy and light chain sequence optimization to improve affinity. Specifically, the sequences of H-CDR1, H-CDR3 and L-CDR1 of Ab89, Ab194 and Ab204 were combined in various combinations to make a series of new Abs (Ab416-Ab424).

The data obtained with this panel of Abs are summarized in Table 4. From these Abs, Ab423 was chosen as candidate for further optimization, based on its properties such as poly-reactivity, predicted immunogenicity and affinity. Ab423 binds to human and cyno CD21 with Biacore K_{D} of 39.9 pM and 233 pM, respectively, indicating 1782- and 92-fold affinity improvement over parental Ab89 (human and cyno K_{D} of 71 nM and 21 nM). Ab423 also inhibits C3d or immune complex binding to Raji B cells or primary B cells. It also showed inhibition in pAKT assay.

TABLE 4
Properties of optimized Ab varients derived from Ab194 and Ab204.

| | | In silico immuno- | | | | | |
|---|---|---|---|---|---|---|---|
| | Poly- | gencicity | | | | pAKT | |
| | reactivity | assessment | | | | inhibition, | |
| | AC- | | # of non- | | | IC50, nM | |
| | SINS, | | germline | Affinity, | | Raji | |
| Ab | DNA, | Epivax | CDR | K_{D}, nM | | B | Primary |
| name | Insulin | score | epitopes | huCD21 | cyCD21 | cells | B cells |
| Ab416 | 4, 1, 2 | −36.97 | 5 | 0.78 | 9.13 | 8.59 | 14.57 |
| Ab417 | 3, 1, 2 | −39.06 | 6 | 0.02 | 0.08 | 8.91 | 3.94 |
| Ab419 | 3, 1, 2 | −39.91 | 6 | 2.38 | 0.21 | 11.20 | 47.77 |
| Ab420 | 15, 2, 2 | −38.89 | 4 | 2.26 | 0.22 | 26.20 | 61.15 |
| Ab421 | 16, 2, 2 | −39.95 | 4 | 2.26 | 0.25 | 8.59 | 56.16 |
| Ab422 | 3, 1, 2 | −37.99 | 7 | 0.04 | 0.24 | 15.81 | 9.98 |
| Ab423 | 8, 2, 2 | −36.97 | 5 | 0.039 | 0.23 | 14.89 | 7.94 |
| Ab424 | 14, 2, 2 | −38.03 | 5 | 0.05 | 0.03 | 9.38 | 11.59 |

While Ab423 has much higher affinity than parental Ab15/Ab89, it has several predicted T-cell epitopes. Based on a crystal structure of an antibody/CD21 complex, a variety of point mutations were designed to remove T-cell epitopes, Ab626-Ab633 (data not shown).

One of those mutants (Ab652) had lower AC-SINS score and maintained high affinity to human and cyno CD21, without having potential T-cell epitopes. Ab652 inhibits immune complex binding to Raji and primary B-cells, as well as in a pAKT assay. Thus, Ab652 was chosen as our final candidate. It is also known as Ab658 (with C-terminal K in HC).

TABLE 5
Characterization of optimized anti-CD21
antibodies to alleviate AC-SINS score.

| | Poly- | Immunogenicity | | | | pAKT | |
|---|---|---|---|---|---|---|---|
| | reactivity | | # of | | | inhibition, | |
| | AC- | | non- | | | IC50, nM | |
| | SINS, | | germline | Affinity, | | Raji | |
| | DNA, | Epivax | CDR | K_{D}, nM | | B | Primary |
| Ab name | Insulin | score | epitopes | huCD21 | cyCD21 | cells | B cells |
| Ab646 | 4, 1, 1 | −40.91 | 2 | 0.01 | 0.05 | nd | nd |
| Ab647 | 12, 2, 1 | −40.94 | 0 | nd | nd | nd | nd |
| Ab648 | 8, 2, 1 | −40.94 | 0 | 0.01 | 0.15 | nd | nd |
| Ab649 | 14, 2, 1 | −38.63 | 0 | nd | nd | nd | nd |
| Ab650 | 18, 2, 1 | −39.89 | 0 | nd | nd | nd | nd |
| Ab651 | 13, 2, 1 | −40.94 | 0 | nd | nd | nd | nd |
| Ab652 | 6, 2, 1 | −39.86 | 0 | 0.013 | 0.14 | nd | 6.31 |
| Ab653 | 18, 2, 1 | −40.94 | 0 | nd | nd | nd | nd |
| Ab658 = | 8, 2, 3 | −39.86 | 0 | 0.011 | 0.12 | nd | 1.66 |
| Ab652 + | | | | | | | |
| CTK | | | | | | | |

*nd: not determined

TABLE 6
Characteristics of ALL optimized anti-CD21 antibodies.

| | Poly- | Immunogenicity | | | | pAKT | |
|---|---|---|---|---|---|---|---|
| | reactivity | | # of | | | inhibition, | |
| | AC- | | non- | | | IC50, nM | |
| | SINS, | | germline | Affinity, | | Raji | |
| | DNA, | Epivax | CDR | K_{D}, nM | | B | Primary |
| Ab name | Insulin | score | epitopes | huCD21 | cyCD21 | cells | B cells |
| Ab416 | 4, 1, 2 | −36.97 | 5 | 0.78 | 9.73 | 8.59 | 14.57 |
| Ab417 | 3, 1, 2 | −39.06 | 6 | 0.02 | 0.08 | 8.91 | 3.94 |
| Ab419 | 3, 1, 2 | −39.91 | 6 | 2.38 | 0.21 | 11.20 | 47.77 |
| Ab420 | 15, 2, 2 | −38.89 | 4 | 2.26 | 0.22 | 26.20 | 61.15 |
| Ab421 | 16, 2, 2 | −39.95 | 4 | 2.26 | 0.25 | 8.59 | 56.16 |
| Ab422 | 3, 1, 2 | −37.99 | 7 | 0.04 | 0.24 | 15.81 | 9.98 |
| Ab423 | 8, 2, 2 | −36.97 | 5 | 0.039 | 0.23 | 14.89 | 7.94 |
| Ab424 | 14, 2, 2 | −38.03 | 5 | 0.05 | 0.03 | 9.38 | 11.59 |
| Ab626 | 18, nd, nd | −40.18 | 2 | nd | nd | nd | nd |
| Ab627 | 20, nd, nd | −40.21 | 2 | 0.02 | 0.15 | nd | nd |
| Ab628 | 16, nd, nd | −40.19 | 2 | 0.01 | 0.11 | nd | 11.65 |
| Ab629 | 20, nd, nd | −40.21 | 2 | 0.02 | 0.1 | nd | nd |
| Ab630 | 20, nd, nd | −39.88 | 0 | 0.01 | 0.09 | nd | nd |
| Ab631 | 22, nd, nd | −39.91 | 0 | 0.03 | 0.19 | nd | nd |
| Ab632 | 16, nd, nd | −39.89 | 0 | 0.01 | 0.08 | nd | 4.43 |
| Ab633 | 21, nd, nd | −39.91 | 0 | 0.02 | 0.12 | nd | nd |
| Ab646 | 4, 1, 1 | −40.91 | 2 | 0.01 | 0.05 | nd | nd |
| Ab647 | 12, 2, 1 | −40.94 | 0 | nd | nd | nd | nd |
| Ab648 | 8, 2, 1 | −40.94 | 0 | 0.01 | 0.15 | nd | nd |
| Ab649 | 14, 2, 1 | −38.63 | 0 | nd | nd | nd | nd |
| Ab650 | 18, 2, 1 | −39.89 | 0 | nd | nd | nd | nd |
| Ab651 | 13, 2, 1 | −40.94 | 0 | nd | nd | nd | nd |
| Ab652 | 6, 2, 1 | −39.86 | 0 | 0.013 | 0.14 | nd | 6.31 |
| Ab653 | 18, 2, 1 | −40.94 | 0 | nd | nd | nd | nd |
| Ab658 | 8, 2, 3 | −39.86 | 0 | 0.011 | 0.12 | nd | 1.66 |

*nd: not determined

TABLE 7
Antibody CDR sequences for exemplary optimized anti-CD21 antibodies

| | SEQ ID NO: | | | | | |
|---|---|---|---|---|---|---|
| Antibody | CDR_H1 | CDR_H2 | CDR_H3 | CDR_L1 | CDR_L2 | CDR_L3 |
| Ab15 | 2 | 3 | 4 | 15 | 16 | 17 |
| Ab89 | 2 | 3 | 4 | 15 | 16 | 17 |
| Ab194 | 30 | 3 | 4 | 33 | 16 | 17 |
| Ab204 | 36 | 3 | 37 | 15 | 16 | 17 |
| Ab414 | 30 | 3 | 4 | 33 | 16 | 17 |
| Ab416 | 2 | 3 | 4 | 33 | 16 | 17 |
| Ab417 | 36 | 3 | 37 | 33 | 16 | 17 |
| Ab419 | 44 | 3 | 37 | 15 | 16 | 17 |
| Ab420 | 2 | 3 | 37 | 15 | 16 | 17 |
| Ab421 | 30 | 3 | 37 | 15 | 16 | 17 |
| Ab422 | 44 | 3 | 37 | 33 | 16 | 17 |
| Ab423 | 2 | 3 | 37 | 33 | 16 | 17 |
| Ab424 | 30 | 3 | 37 | 33 | 16 | 17 |
| Ab626 | 2 | 3 | 51 | 54 | 16 | 55 |
| Ab627 | 2 | 3 | 51 | 59 | 16 | 55 |
| Ab628 | 2 | 3 | 51 | 62 | 16 | 55 |
| Ab629 | 2 | 3 | 51 | 65 | 16 | 55 |
| Ab630 | 2 | 3 | 51 | 54 | 68 | 55 |
| Ab631 | 2 | 3 | 51 | 59 | 68 | 55 |
| Ab632 | 2 | 3 | 51 | 62 | 68 | 55 |
| Ab633 | 2 | 3 | 51 | 65 | 68 | 55 |
| Ab646 | 44 | 3 | 51 | 54 | 68 | 55 |
| Ab647 | 81 | 3 | 51 | 54 | 68 | 55 |
| Ab648 | 84 | 3 | 51 | 54 | 68 | 55 |
| Ab649 | 87 | 3 | 51 | 54 | 68 | 55 |
| Ab650 | 90 | 3 | 51 | 54 | 68 | 55 |
| Ab651 | 93 | 3 | 51 | 54 | 68 | 55 |
| Ab652 | 96 | 3 | 51 | 54 | 68 | 55 |
| Ab653 | 99 | 3 | 51 | 54 | 68 | 55 |
| Ab658 | 96 | 3 | 51 | 54 | 68 | 55 |

TABLE 8
Antibody CH1, CH2, CH3, Linker and CL Sequences
for optimized anti-CD21 antibodies

| | SEQ ID NO: | | | | |
|---|---|---|---|---|---|
| Antibody | CH1 | CH1_CH2_HINGE | CH2 | CH3 | CL |
| Ab15 | 5 | 6 | 7 | 8 | 18 |
| Ab89 | 5 | 6 | 7 | 8 | 18 |
| Ab194 | 5 | 6 | 7 | 8 | 18 |
| Ab204 | 5 | 6 | 7 | 8 | 18 |
| Ab414 | 5 | 6 | 7 | 40 | 18 |
| Ab416 | 5 | 6 | 7 | 40 | 18 |
| Ab417 | 5 | 6 | 7 | 40 | 18 |
| Ab419 | 5 | 6 | 7 | 40 | 18 |
| Ab420 | 5 | 6 | 7 | 40 | 18 |
| Ab421 | 5 | 6 | 7 | 40 | 18 |
| Ab422 | 5 | 6 | 7 | 40 | 18 |
| Ab423 | 5 | 6 | 7 | 40 | 18 |
| Ab424 | 5 | 6 | 7 | 40 | 18 |
| Ab626 | 5 | 6 | 7 | 40 | 18 |
| Ab627 | 5 | 6 | 7 | 40 | 18 |
| Ab628 | 5 | 6 | 7 | 40 | 18 |
| Ab629 | 5 | 6 | 7 | 40 | 18 |
| Ab630 | 5 | 6 | 7 | 40 | 18 |
| Ab631 | 5 | 6 | 7 | 40 | 18 |
| Ab632 | 5 | 6 | 7 | 40 | 18 |
| Ab633 | 5 | 6 | 7 | 40 | 18 |
| Ab646 | 5 | 6 | 7 | 40 | 18 |
| Ab647 | 5 | 6 | 7 | 40 | 18 |
| Ab648 | 5 | 6 | 7 | 40 | 18 |
| Ab649 | 5 | 6 | 7 | 40 | 18 |
| Ab650 | 5 | 6 | 7 | 40 | 18 |
| Ab651 | 5 | 6 | 7 | 40 | 18 |
| Ab652 | 5 | 6 | 7 | 40 | 18 |
| Ab653 | 5 | 6 | 7 | 40 | 18 |
| Ab658 | 5 | 6 | 7 | 8 | 18 |

TABLE 9
Antibody HC and LC FV and Framework Sequences for exemplary anti-CD21 antibodies

| | SEQ ID NO: | | | | | | | | | |
|---|---|---|---|---|---|---|---|---|---|---|
| Antibody | FV_H | FV_L | FW_H1 | FW_H2 | FW_H3 | FW_H4 | FW_L1 | FW_L2 | FW_L3 | FW_L4 |
| Ab15 | 9 | 19 | 10 | 11 | 12 | 13 | 20 | 21 | 23 | 23 |
| Ab89 | 9 | 25 | 10 | 11 | 12 | 13 | 26 | 27 | 29 | 23 |
| Ab194 | 31 | 34 | 10 | 11 | 12 | 13 | 26 | 27 | 29 | 23 |
| Ab204 | 38 | 25 | 10 | 11 | 12 | 13 | 26 | 27 | 29 | 23 |
| Ab414 | 31 | 34 | 10 | 11 | 12 | 13 | 26 | 27 | 29 | 23 |
| Ab416 | 9 | 34 | 10 | 11 | 12 | 13 | 26 | 27 | 29 | 23 |
| Ab417 | 38 | 34 | 10 | 11 | 12 | 13 | 26 | 27 | 29 | 23 |
| Ab419 | 45 | 25 | 10 | 11 | 12 | 13 | 26 | 27 | 29 | 23 |
| Ab420 | 47 | 25 | 10 | 11 | 12 | 13 | 26 | 27 | 29 | 23 |
| Ab421 | 49 | 25 | 10 | 11 | 12 | 13 | 26 | 27 | 29 | 23 |
| Ab422 | 45 | 34 | 10 | 11 | 12 | 13 | 26 | 27 | 29 | 23 |
| Ab423 | 47 | 34 | 10 | 11 | 12 | 13 | 26 | 27 | 29 | 23 |
| Ab424 | 49 | 34 | 10 | 11 | 12 | 13 | 26 | 27 | 29 | 23 |
| Ab626 | 52 | 56 | 10 | 11 | 12 | 13 | 57 | 27 | 29 | 23 |
| Ab627 | 52 | 60 | 10 | 11 | 12 | 13 | 26 | 27 | 29 | 23 |
| Ab628 | 52 | 63 | 10 | 11 | 12 | 13 | 26 | 27 | 29 | 23 |
| Ab629 | 52 | 66 | 10 | 11 | 12 | 13 | 26 | 27 | 29 | 23 |
| Ab630 | 52 | 69 | 10 | 11 | 12 | 13 | 57 | 27 | 29 | 23 |
| Ab631 | 52 | 71 | 10 | 11 | 12 | 13 | 26 | 27 | 29 | 23 |
| Ab632 | 52 | 73 | 10 | 11 | 12 | 13 | 26 | 27 | 29 | 23 |
| Ab633 | 52 | 75 | 10 | 11 | 12 | 13 | 26 | 27 | 29 | 23 |
| Ab646 | 77 | 79 | 10 | 11 | 12 | 13 | 26 | 27 | 29 | 23 |
| Ab647 | 82 | 79 | 10 | 11 | 12 | 13 | 26 | 27 | 29 | 23 |
| Ab648 | 85 | 79 | 10 | 11 | 12 | 13 | 26 | 27 | 29 | 23 |
| Ab649 | 88 | 79 | 10 | 11 | 12 | 13 | 26 | 27 | 29 | 23 |
| Ab650 | 91 | 79 | 10 | 11 | 12 | 13 | 26 | 27 | 29 | 23 |
| Ab651 | 94 | 79 | 10 | 11 | 12 | 13 | 26 | 27 | 29 | 23 |
| Ab652 | 97 | 79 | 10 | 11 | 12 | 13 | 26 | 27 | 29 | 23 |
| Ab653 | 100 | 79 | 10 | 11 | 12 | 13 | 26 | 27 | 29 | 23 |
| Ab658 | 97 | 79 | 10 | 11 | 12 | 13 | 26 | 27 | 29 | 23 |

TABLE 10
Antibody heavy chain and light chain sequences
for exemplary anti-CD21 antibodies

SEQ ID NOS:

| Antibody | H | L |
|---|---|---|
| Ab15 | 1 | 24 |
| Ab89 | 1 | 32 |
| Ab194 | 29 | 32 |
| Ab204 | 35 | 24 |
| Ab414 | 39 | 32 |
| Ab416 | 41 | 32 |
| Ab417 | 42 | 32 |
| Ab419 | 43 | 24 |
| Ab420 | 46 | 24 |
| Ab421 | 48 | 24 |
| Ab422 | 43 | 32 |
| Ab423 | 46 | 32 |
| Ab424 | 48 | 32 |
| Ab626 | 50 | 53 |
| Ab627 | 50 | 58 |
| Ab628 | 50 | 61 |
| Ab629 | 50 | 64 |
| Ab630 | 50 | 67 |
| Ab631 | 50 | 70 |
| Ab632 | 50 | 72 |
| Ab633 | 50 | 74 |
| Ab646 | 76 | 78 |
| Ab647 | 80 | 78 |
| Ab648 | 83 | 78 |
| Ab649 | 86 | 78 |
| Ab650 | 89 | 78 |
| Ab651 | 92 | 78 |
| Ab652 | 95 | 78 |
| Ab653 | 98 | 78 |
| Ab658 | 101 | 78 |

### Development of CD21 Antibodies

The anti-CD21 IgG1 antibodies described herein were designed to block the binding of C3d-coated immune complexes to B cells and follicular dendritic cells (FDCs) and block the CD21/C3d-mediated functions in these cells to ablate GC, decrease pathogenic auto-antibodies and reduce the autoimmune response in SLE. The antibodies were designed to be an IgG1 with three mutations in the Fc domain to abrogate effector function. Antibodies against CD21 were developed utilizing Pfizer's phage display libraries and/or by hybridoma technology. The C3d binding domain of CD21 resides in the 2 most N-terminal short consensus repeats (SCR) of CD21 and has been well characterized. In addition, antibodies against human or murine SCR1-2 or a Ig-Fc fusion protein of human SCR1-2 have been shown to block binding to C3d ex-vivo and in-vivo. Therefore, the CD21 (SCR1-2) (I1-Val128) domain protein was initially utilized for panning and library screening.

scFVs were screened for binding cross-reactivity to mouse and cynomolgus monkey SCR1-2 domains. Identity with the corresponding human and mouse SCR1-2 proteins is 82% and 60% respectively (identity for full length CD21 of cynomolgus monkey and mouse are 90 and 62% respectively). Crossreactivity to cynomolgus monkey is required and crossreactivity to mouse is highly desirable for target engagement and PK/PD studies in mice.

CD21 in humans is expressed on B cells and FDCs. Binding to endogenous CD21 can be demonstrated by flow cytometry: 1) to Raji cells, a human B cell line that expresses CD21; 2) peripheral blood human B cells, and 3) cultures of differentiated or isolated human follicular dendritic cells (e.g. human FDCs isolated from human tonsil). In addition, specificity of binding can be demonstrated by lack of binding to other leukocyte subsets (neutrophils, monocytes, T cells) as B cells and PDCs are the only two cell types that express CD21.

Early on during primary screening efforts, specificity of binding can be demonstrated by lack of crossreactivity to CR1. Binding to CR1 can be measured by binding to the recombinant SCR1-2 domain of CR1. Binding reactivity to the endogenous CR1 protein can be evaluated by flow cytometry as the CR1 receptor is expressed on the surface of erythrocytes, neutrophils and monocytes, cells which do not express CD21.

Blocking activity can be determined in the following assays: 1) binding of recombinant SCR1-2 to recombinant C3d and the 2 other C3 ligands of CR2, the intermediate degradation products iC3b and C3dg, reported to bind with similar affinity as C3d. 2) binding of immune complex of PE and anti PE antibody in the presence of human serum to Raji cells, a human B cell line that expresses CR2 3) binding of immune complex of PE and anti PE antibody in the presence of human serum to human B cells and 4) removal of immune complex bound to circulating B cells from lupus patients. The complexes on B cells can be detected with antibodies against C3d. Given that a main function of CD21 is the uptake and retention of immune complexes, removal of immune complexes from the surface of B cells is considered a surrogate ex-vivo functional assay. In addition, candidate antibodies can be evaluated for their ability to decrease BCR hyperactivity by increasing the threshold of activation in B cell activation assays (e.g. Ca2+ flux or proliferation assays)

Physiological Kds can be determined from binding and blocking studies in whole human blood. Ex vivo experiments can determine trough blood concentrations of antibody required to achieve saturable target coverage on B cells. This data will provide an understanding of the required dosing regimen for a given antibody.

The fusion protein SCR1-2-IgG1(Fc) is recombinantly produced and used as the benchmark standard for all these assays. Antibodies with the same or greater potency as this protein were selected. The reported affinity of (SCR1-2)2 for multimeric C3d is in the range of 6-10 nM.

### Human Clinical Trials: SLE

### Biomarker Characterization for Patient Stratification

Anti-dsDNA antibodies in SLE patients likely mediate kidney tissue injury in lupus nephritis patients. In addition, although the correlation of serum complement levels to disease severity or to flare or quiescent disease is controversial, C3d in urine is reported to correlate to kidney tissue injury. Therefore, lupus nephritis patients will be studied first, to determine which patient populations are eligible for CD21 therapies. Other manifestations of lupus strongly linked to autoantibodies such as vascular complications characterized by cytopenias mediated by antibodies against leukocyte targets will be studied.

Lupus patients are screened for the presence of auto-reactive antibodies, immune complexes and other serum (or urine) biomarkers including complement proteins, or CR2 levels on B cells, and can also be evaluated for the presence of immune complexes bound to circulating B cells in an effort to understand the feasibility of performing the POM study described below and for selection of potential patients.

First, the efficacy of a CD21 antibody can be determined using a standard Phase 1 safety, tolerability pharmacokinetics (PK) pharmacodynamics (PD) study in lupus nephritis patients. The CD21 role in FDC retention of self-antigen is important for maintenance of auto-reactive germinal centers. Without wishing to be bound by theory, the inventors hypothesize that disrupting this pathway will severely limit antibody responses, and that this will be reflected in a decrease in circulating auto-reactive antibodies. Patients are monitored for the ability of the therapeutic antibody to detach complement-mediated immune complex from the surface of circulating B cells, for reductions in auto-antibodies titers, particularly anti-dsDNA antibodies, and for kidney function.

Inclusion criteria: Patients are screened for the presence of complement-IC on their B cells and for the ability of the therapeutic antibody to detach them in ex vivo assays. SLE patients with elevated levels of anti-dsDNA autoantibodies (or other relevant pathogenic antibodies) presence of circulating immune complexes and immune complexes bound to the surface of circulating B cells will be prioritized for entry into the study. Urine C3d levels will also be measured. In addition, fulfillment of the following two clinical and one of the following two laboratory criteria are proposed for inclusion in a lupus nephritis trial (Weening, 2004):
[1] Diagnosis of SLE according to the American College of Rheumatology (ACR) criteria;
[2] Kidney biopsy within the 6 months of enrollment with a histologic diagnosis of lupus nephritis;
[3A] Proteinuria≥1000 mg/24 h; or
[3B] Serum creatinine above 1.3 mg/dL (115 μmol/L).

Primary mechanistic endpoints of POM study: Removal of immune complexes from the surface of circulating B cells.

Secondary endpoints of POM study: Reduction in anti-ds-DNA

Other biomarkers associated with immune activation and SLE will be monitored including:
a. Gene expression signatures in peripheral blood cells (type I interferon (IFN-1) gene signatures, neutrophil-granule genes)
b. MicroRNAs (miRNAs) found to be differentially expressed on PBMCs of patients with SLE as compared to controls as well as miRNAs found to be differentially expressed on T and B cells from patients with SLE.
c. Protein biomarkers including IP-10, vWF, CRP, TNFRII, which have been shown to be associated with SLE flares, as well as other typical laboratory measures that are part of the SLE Disease Activity Index (SLEDAI) including complement, dsDNA antibodies etc.

For further proof of concept studies to demonstrate clinically meaningful improvement in disease signs and symptoms a significant clinical response is defined as:
a. Decrease in proteinuria, defined as decrease in the urine protein/creatinine ratio to <3 in subjects with baseline nephrotic range proteinuria (≥3 urine protein/creatinine ratio) or decrease in the urine protein/creatinine ratio by ≥50% in subjects with sub-nephrotic proteinuria (<3 urine protein/creatinine ratio). This ratio is based on the 24 hour urine collection.
b. Stabilization of serum creatinine (i.e., serum creatinine level ±25% of baseline)

Complete Renal remission is defined as:
I. Return to normal serum creatinine,
II. Proteinuria ≤500 mg/24 hours
III. Inactive urinary sediment.

What is claimed is:

1. An antibody or antigen binding fragment that binds to CD21 and comprises:
a) a heavy chain variable region (V_{H}) comprising:
i. a complementarity determining region 1 (CDR_H1) sequence comprising an amino acid sequence that differs by no more than four amino acids from the amino acid sequence G-G-X₁-X₂-X₃-X₄-X₅-A-I-S (SEQ ID NO: 102), wherein X₁ is T or E; X₂ is F or L; X₃ is S, or Q; X₄ is T, D, or S; X₅ is Y or L;
ii. a complementarity determining region 2 (CDR_H2) sequence comprising an amino acid sequence that differs by no more than four amino acids from the amino acid sequence RIIPILGIANYAQKFQG (SEQ ID NO: 3); or
iii. a complementarity determining region 3 (CDR_H3) sequence comprising an amino acid sequence that differs by no more than four amino acids from the amino acid sequence E-X₆-D-X₇-S-G-Y-Y-Q-D (SEQ ID NO: 103), wherein X₆ is D or E; X₇ is S or I; or
b) a light chain variable region (V_{L}) comprising:
i. a complementarity determining region 1 (CDR_L1) sequence comprising an amino acid sequence that differs by no more than four amino acids from the amino acid sequence R-A-G-Q-X₈-I-X₉-X₁₀-Y-L-A (SEQ ID NO: 104), wherein X₈ is S or K; X₉ is N or S; X₁₀ is S, H, or R;
ii. a complementarity determining region 2 (CDR_L2) sequence comprising an amino acid sequence that differs by no more than four amino acids from the amino acid sequence D-A-S-X₁₁-R-A-T (SEQ ID NO: 105), wherein X₁₁ is S or T; or
iii. a complementarity determining region 3 (CDR_L3) sequence comprising an amino acid sequence that differs by no more than four amino acids from the amino acid sequence Q-Q-Y-E-Y-W-Y-X₁₂ (SEQ ID NO: 106), wherein X₁₂ is S or T.

2. The antibody or antigen binding fragment of claim 1, wherein the antibody or antigen-binding fragment comprises a nanobody, an scFv, a monoclonal antibody, a humanized antibody, a human antibody, a recombinant antibody, a chimeric antibody, or a Fab fragment.

3. The antibody or antigen binding fragment of claim 1 or 2, wherein the CDR_H1 comprises an amino acid sequence that differs by no more than four amino acids from an amino acid sequence selected from the group consisting of: SEQ ID NOs.: 2, 30, 36, 44, 81, 84, 87, 90, 93, 96, and 99.

4. The antibody or antigen binding fragment of any one of claims 1-3, wherein the CDR_H2 comprises an amino acid sequence that differs by no more than four amino acids from SEQ ID NO: 3.

5. The antibody or antigen binding fragment of any one of claims 1-4, wherein the CDR_H3 comprises an amino acid sequence that differs by no more than four amino acids from an amino acid sequence selected from the group consisting of: SEQ ID NOs.: 4, 37, and 51.

6. The antibody or antigen binding fragment of any one of claims 1-5, wherein the CDR_L1 comprises an amino acid sequence that differs by no more than four amino acids from an amino acid sequence selected from the group consisting of: SEQ ID NOs.: SEQ ID NOS: 15, 33, 54, 59, 62 and 65.

7. The antibody or antigen binding fragment of any one of claims 1-6, wherein the CDR_L2 comprises an amino acid sequence that differs by no more than four amino acids from an amino acid sequence selected from the group consisting of: SEQ ID NOs.: 16 and 68.

8. The antibody or antigen binding fragment of any one of claims 1-7, wherein the CDR_L3 comprises an amino acid sequence that differs by no more than four amino acids from an amino acid sequence selected from the group consisting of: SEQ ID NOs.: 17 and 55.

9. The antibody or antigen binding fragment of any one of claims 1-8, wherein CDR_H1 comprises an amino acid sequence that differs by no more than four amino acids from SEQ ID NO: 2, CDR_H2 comprises an amino acid sequence that differs by no more than four amino acids from SEQ ID NO: 3, and CDR_H3 comprises an amino acid sequence that differs by no more than four amino acids from SEQ ID NO: 4; or

10. The antibody or antigen binding fragment of any one of claims 1-8, wherein CDR_H1 comprises an amino acid sequence that differs by no more than four amino acids from SEQ ID NO: 30, CDR_H2 comprises an amino acid sequence that differs by no more than four amino acids from SEQ ID NO: 3, and CDR_H3 comprises an amino acid sequence that differs by no more than four amino acids from SEQ ID NO: 4; or

11. The antibody or antigen binding fragment of any one of claims 1-8, wherein CDR_H1 comprises an amino acid sequence that differs by no more than four amino acids from SEQ ID NO: 36, CDR_H2 comprises an amino acid sequence that differs by no more than four amino acids from SEQ ID NO: 3, and CDR_H3 comprises an amino acid sequence that differs by no more than four amino acids from SEQ ID NO: 37; or

12. The antibody or antigen binding fragment of any one of claims 1-8, wherein CDR_H1 comprises an amino acid sequence that differs by no more than four amino acids from SEQ ID NO: 44, CDR_H2 comprises an amino acid sequence that differs by no more than four amino acids from SEQ ID NO: 3, and CDR_H3 comprises an amino acid sequence that differs by no more than four amino acids from SEQ ID NO: 37; or

13. The antibody or antigen binding fragment of any one of claims 1-8, wherein CDR_H1 comprises an amino acid sequence that differs by no more than four amino acids from SEQ ID NO: 2, CDR_H2 comprises an amino acid sequence that differs by no more than four amino acids from SEQ ID NO: 3, and CDR_H3 comprises an amino acid sequence that differs by no more than four amino acids from SEQ ID NO: 37; or

14. The antibody or antigen binding fragment of any one of claims 1-8, wherein CDR_H1 comprises an amino acid sequence that differs by no more than four amino acids from SEQ ID NO: 30, CDR_H2 comprises an amino acid sequence that differs by no more than four amino acids from SEQ ID NO: 3, and CDR_H3 comprises an amino acid sequence that differs by no more than four amino acids from SEQ ID NO: 37; or

15. The antibody or antigen binding fragment of any one of claims 1-8, wherein CDR_H1 comprises an amino acid sequence that differs by no more than four amino acids from SEQ ID NO: 2, CDR_H2 comprises an amino acid sequence that differs by no more than four amino acids from SEQ ID NO: 3, and CDR_H3 comprises an amino acid sequence that differs by no more than four amino acids from SEQ ID NO: 51; or

16. The antibody or antigen binding fragment of any one of claims 1-8, wherein CDR_H1 comprises an amino acid sequence that differs by no more than four amino acids from SEQ ID NO: 44, CDR_H2 comprises an amino acid sequence that differs by no more than four amino acids from SEQ ID NO: 3, and CDR_H3 comprises an amino acid sequence that differs by no more than four amino acids from SEQ ID NO: 51; or

17. The antibody or antigen binding fragment of any one of claims 1-8, wherein CDR_H1 comprises an amino acid sequence that differs by no more than four amino acids from SEQ ID NO: 81, CDR_H2 comprises an amino acid sequence that differs by no more than four amino acids from SEQ ID NO: 3, and CDR_H3 comprises an amino acid sequence that differs by no more than four amino acids from SEQ ID NO: 51; or

18. The antibody or antigen binding fragment of any one of claims 1-8, wherein CDR_H1 comprises an amino acid sequence that differs by no more than four amino acids from SEQ ID NO: 84, CDR_H2 comprises an amino acid sequence that differs by no more than four amino acids from SEQ ID NO: 3, and CDR_H3 comprises an amino acid sequence that differs by no more than four amino acids from SEQ ID NO: 51; or

19. The antibody or antigen binding fragment of any one of claims 1-8, wherein CDR_H1 comprises an amino acid sequence that differs by no more than four amino acids from SEQ ID NO: 87, CDR_H2 comprises an amino acid sequence that differs by no more than four amino acids from SEQ ID NO: 3, and CDR_H3 comprises an amino acid sequence that differs by no more than four amino acids from SEQ ID NO: 51; or

20. The antibody or antigen binding fragment of any one of claims 1-8, wherein CDR_H1 comprises an amino acid sequence that differs by no more than four amino acids from SEQ ID NO: 90, CDR_H2 comprises an amino acid sequence that differs by no more than four amino acids from SEQ ID NO: 3, and CDR_H3 comprises an amino acid sequence that differs by no more than four amino acids from SEQ ID NO: 51; or

21. The antibody or antigen binding fragment of any one of claims 1-8, wherein CDR_H1 comprises an amino acid sequence that differs by no more than four amino acids from SEQ ID NO: 93, CDR_H2 comprises an amino acid sequence that differs by no more than four amino acids from SEQ ID NO: 3, and CDR_H3 comprises an amino acid sequence that differs by no more than four amino acids from SEQ ID NO: 51; or

22. The antibody or antigen binding fragment of any one of claims 1-8, wherein CDR_H1 comprises an amino acid sequence that differs by no more than four amino acids from SEQ ID NO: 96, CDR_H2 comprises an amino acid sequence that differs by no more than four amino acids from SEQ ID NO: 3, and CDR_H3 comprises an amino acid sequence that differs by no more than four amino acids from SEQ ID NO: 51; or

23. The antibody or antigen binding fragment of any one of claims 1-8, wherein CDR_H1 comprises an amino acid sequence that differs by no more than four amino acids from SEQ ID NO: 99, CDR_H2 comprises an amino acid sequence that differs by no more than four amino acids from SEQ ID NO: 3, and CDR_H3 comprises an amino acid sequence that differs by no more than four amino acids from SEQ ID NO: 51.

24. The antibody or antigen binding fragment of any one of claims 1-23, wherein CDR_L1 comprises an amino acid sequence that differs by no more than four amino acids from SEQ ID NO: 15, CDR_L2 comprises an amino acid sequence that differs by no more than four amino acids from SEQ ID NO: 16, and CDR_L3 comprises an amino acid sequence that differs by no more than four amino acids from SEQ ID NO: 17.

25. The antibody or antigen binding fragment of any one of claims 1-23, wherein CDR_L1 comprises an amino acid sequence that differs by no more than four amino acids from SEQ ID NO: 33, CDR_L2 comprises an amino acid sequence that differs by no more than four amino acids from SEQ ID NO: 16, and CDR_L3 comprises an amino acid sequence that differs by no more than four amino acids from SEQ ID NO: 17.

26. The antibody or antigen binding fragment of any one of claims 23, wherein CDR_L1 comprises an amino acid sequence that differs by no more than four amino acids from SEQ ID NO: 54, CDR_L2 comprises an amino acid sequence that differs by no more than four amino acids from SEQ ID NO: 16, and CDR_L3 comprises an amino acid sequence that differs by no more than four amino acids from SEQ ID NO: 55.

27. The antibody or antigen binding fragment of any one of claims 23, wherein CDR_L1 comprises an amino acid sequence that differs by no more than four amino acids from SEQ ID NO: 59, CDR_L2 comprises an amino acid sequence that differs by no more than four amino acids from SEQ ID NO: 16, and CDR_L3 comprises an amino acid sequence that differs by no more than four amino acids from SEQ ID NO: 55.

28. The antibody or antigen binding fragment of any one of claims 23, wherein CDR_L1 comprises an amino acid sequence that differs by no more than four amino acids from SEQ ID NO: 62, CDR_L2 comprises an amino acid sequence that differs by no more than four amino acids from SEQ ID NO: 16, and CDR_L3 comprises an amino acid sequence that differs by no more than four amino acids from SEQ ID NO: 55.

29. The antibody or antigen binding fragment of any one of claims 23, wherein CDR_L1 comprises an amino acid sequence that differs by no more than four amino acids from SEQ ID NO: 65, CDR_L2 comprises an amino acid sequence that differs by no more than four amino acids from SEQ ID NO: 16, and CDR_L3 comprises an amino acid sequence that differs by no more than four amino acids from SEQ ID NO: 55.

30. The antibody or antigen binding fragment of any one of claims 23, wherein CDR_L1 comprises an amino acid sequence that differs by no more than four amino acids from SEQ ID NO: 54, CDR_L2 comprises an amino acid sequence that differs by no more than four amino acids from SEQ ID NO: 68, and CDR_L3 comprises an amino acid sequence that differs by no more than four amino acids from SEQ ID NO: 55.

31. The antibody or antigen binding fragment of any one of claims 23, wherein CDR_L1 comprises an amino acid sequence that differs by no more than four amino acids from SEQ ID NO: 59, CDR_L2 comprises an amino acid sequence that differs by no more than four amino acids from SEQ ID NO: 68, and CDR_L3 comprises an amino acid sequence that differs by no more than four amino acids from SEQ ID NO: 55.

32. The antibody or antigen binding fragment of any one of claims 23, wherein CDR_L1 comprises an amino acid sequence that differs by no more than four amino acids from SEQ ID NO: 62, CDR_L2 comprises an amino acid sequence that differs by no more than four amino acids from SEQ ID NO: 68, and CDR_L3 comprises an amino acid sequence that differs by no more than four amino acids from SEQ ID NO: 55.

33. The antibody or antigen binding fragment of any one of claims 23, wherein CDR_L1 comprises an amino acid sequence that differs by no more than four amino acids from SEQ ID NO: 65, CDR_L2 comprises an amino acid sequence that differs by no more than four amino acids from SEQ ID NO: 68, and CDR_L3 comprises an amino acid sequence that differs by no more than four amino acids from SEQ ID NO: 55.

34. The antibody or antigen binding fragment of any one of claims 1-33, wherein the heavy chain variable region further comprises a Framework region 1 (FW_H1) sequence that differs by no more than four, preferably no more than three, more preferably no more than two, most preferably no more than one amino acid from SEQ ID NO: 10.

35. The antibody or antigen binding fragment of any one of claims 1-34, wherein the heavy chain variable region further comprises a Framework region 2 (FW_H2) sequence that differs by no more than four, preferably no more than three, more preferably no more than two, most preferably no more than one amino acid from SEQ ID NO: 11.

36. The antibody or antigen binding fragment of any one of claims 1-35, wherein the heavy chain variable region further comprises a Framework region 3 (FW_H3) sequence that differs by no more than four, preferably no more than three, more preferably no more than two, most preferably no more than one amino acid from SEQ ID NO: 12.

37. The antibody or antigen binding fragment of any one of claims 1-36, wherein the heavy chain variable region further comprises a Framework region 4 (FW_H4) sequence that differs by no more than four, preferably no more than three, more preferably no more than two, most preferably no more than one amino acid from SEQ ID NO: 13.

38. The antibody or antigen binding fragment of any one of claims 1-37, wherein the heavy chain variable region comprises an amino acid sequence having at least 85%, identity to an amino acid sequence selected from the group consisting of SEQ ID NOs: 9, 31, 38, 45, 47, 49, 52, 77, 82, 85, 88, 91, 94, 97 and 100.

39. The antibody or antigen binding fragment of any one of claims 1-38, wherein the antibody or antigen binding fragment comprises a heavy chain constant region 1 (CH1) comprising an amino acid sequence having at least 85% identity to SEQ ID NO: 5.

40. The antibody or antigen binding fragment of any one of claims 1-39, wherein the antibody or antigen binding fragment comprises a heavy chain constant region 2 (CH2) comprising an amino acid sequence having at least 85% identity to SEQ ID NO: 7.

41. The antibody or antigen binding fragment of any one of claims 1-40, wherein the antibody or antigen binding fragment comprises a heavy chain constant region 1 (CH3) comprising an amino acid sequence having at least 85% identity to SEQ ID NO: 8 or 40.

42. The antibody or antigen binding fragment of any one of claims 1-41, wherein the antibody or antigen binding fragment comprises a heavy chain hinge region (CH1_CH2_hinge) comprising an amino acid sequence having at least 85% identity to SEQ ID NO: 6.

43. The antibody or antigen binding fragment of any one of claims 1-42, wherein the antibody or antigen-binding fragment comprises a heavy chain amino acid sequence having at least 85% identity to an amino acid sequence selected from the group consisting of SEQ ID NOs: 1, 29, 35, 39, 41, 42, 43, 46, 48, 50, 76, 80, 83, 86, 89, 92, 95, 98 and 101.

44. The antibody or antigen binding fragment of any one of claims 1-43, wherein the light chain variable region further comprises a Framework region 1 (FW_L1) sequence that differs by no more than four amino acids from SEQ ID NO: 20, 26 or 57.

45. The antibody or antigen binding fragment of any one of claims 1-44, wherein the light chain variable region further comprises a Framework region 2 (FW_L2) sequence that differs by no more than four amino acids from SEQ ID NO: 21 or 27.

46. The antibody or antigen binding fragment of any one of claims 1-45, wherein the light chain variable region further comprises a Framework region 3 (FW_L3) sequence that differs by no more than four amino acids from SEQ ID NO: 23 or 29.

47. The antibody or antigen binding fragment of any one of claims 1-46, wherein the light chain variable region further comprises a Framework region 4 (FW_L4) sequence that differs by no more than four amino acids from SEQ ID NO: 23.

48. The antibody or antigen binding fragment of any one of claims 1-47, wherein the light chain variable region comprises an amino acid sequence having at least 85% identity to an amino acid sequence selected from the group consisting of SEQ ID NOs: 19, 25, 34, 56, 60, 63, 66, 69, 71, 73, 75 and 79.

49. The antibody or antigen binding fragment of any one of claims 1-48, wherein the antibody or antigen binding fragment comprises a light chain constant region (C_{L}) comprising an amino acid sequence having at least 85% identity to SEQ ID NO: 18.

50. The antibody or antigen binding fragment of any one of claims 1-49, wherein the antibody or antigen binding fragment a light chain amino acid sequence having at least 85% identity to an amino acid sequence selected from the group consisting of SEQ ID NOs: 24, 32, 53, 58, 61, 64, 67, 70, 72, 74 and 78.

51. The antibody or antigen binding fragment of any one of claims 1-50, wherein the antibody or antigen binding fragment is a CD21 inhibitor or a CD21 neutralizing antibody.

52. The antibody or antigen binding fragment of any one of claims 1-51, wherein the antibody or antigen binding fragment reduces CD21 activity and/or B cell activation by at least 20% as compared to CD21 activity and/or B cell activation in the absence of the antibody or antigen-binding fragment.

53. The antibody or antigen binding fragment of any one of claims 1-52, wherein the antibody or antigen binding fragment disrupts binding of CD21 to its cognate ligand C3d.

54. A composition comprising an antibody or antigen binding fragment of any one of claims 1-53.

55. The composition of claim 54, wherein the composition comprises a pharmaceutically acceptable excipient or carrier.

56. A cell comprising an antibody or antigen binding fragment of any one of claims 1-53.

57. A polynucleotide comprising a nucleotide sequence encoding an antibody or antigen binding fragment of any one of claims 1-53.

58. The polynucleotide of claim 57, wherein the polynucleotide is comprised in a vector.

59. A composition comprising a polynucleotide claim 57 or 58.

60. A cell comprising a polynucleotide of claim 57 or 58.

61. A kit comprising:
a) an antibody or antigen binding fragment of any one of claims 1-53;
b) a polynucleotide of claim 57 or 58;
c) a composition of claim 54, 55, 58 or 59; or
d) a cell of claim 56 or 60.

62. The kit of claim 61, further comprising instructions for use thereof.

63. A method for reducing CD21 activity or CD21/C3d mediated B cell activation, the method comprising: administering an antibody or antigen-binding fragment of any one of claims 1-53 or a polynucleotide of claim 57 or 58 to a B cell.

64. The method of claim 63, wherein said administering to the B cell is in vitro.

65. The method of claim 63, wherein said administering to the B cell is in vivo.

66. The method of claim 65, wherein said administering to the cell is in a subject diagnosed with or in need of treatment for an autoimmune disease or disorder.

67. A method for reducing at least one symptom of an autoimmune disease or disorder, the method comprising administering a therapeutically effective amount of an antibody or antigen-binding fragment of any one of claims 1-53 or a polynucleotide of claim 57 or 58 to a subject in need thereof.

68. The method of claim 67, wherein the autoimmune disease or disorder comprises systemic lupus erythematosus (SLE).

69. The method of claim 68, wherein the at least one symptom of SLE comprises lupus nephritis, malar rash, discoid rash, butterfly rash, photosensitivity, oral ulcers, arthritis, serositis, pleuritis, pericarditis, proteinuria, seizures, psychosis, hemolytic anemia, leukopenia, lymphopenia, or thrombocytopenia.

70. The method of claim 68 or 69, wherein the at least one symptom of SLE comprises a clinical measure of SLE selected from the group consisting of: positive lupus erythematosus cell preparation, anti-DNA antibody to native DNA ratio, and anti-SM nuclear antigen antibodies.

71. The method of any one of claims 65-70 further comprising a step of diagnosing the subject with the autoimmune disease or disorder.

72. The method of any one of claims 65-71, wherein the subject is a mammal.

73. The method of any one of claims 65-71, wherein the subject is human.

74. A composition of any one of claims 1-60 for use in the treatment of an autoimmune disease or disorder, or at least one symptom thereof in a subject.


SEQUENCE LISTING (134 sequences)

SEQ ID NO: 1 - protein, 449 aa, synthetic construct
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCAREDDSSGYYQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 10 aa, synthetic construct
GGTFSSYAIS

SEQ ID NO: 3 - protein, 17 aa, synthetic construct
RIIPILGIANYAQKFQG

SEQ ID NO: 4 - protein, 10 aa, synthetic construct
EDDSSGYYQD

SEQ ID NO: 5 - protein, 97 aa, synthetic construct
ASTKGPSVFPLAPSSKSTSGGTAALGCLVKDYFPEPVTVSWNSGALTSGVHTFPAVLQSSGLYSLSSVVTVPSSSLGTQTICNVNHKPSNTKVDKKV

SEQ ID NO: 6 - protein, 15 aa, synthetic construct
EPKSCDKTHTCPPCP

SEQ ID NO: 7 - protein, 110 aa, synthetic construct
APEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 8 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 119 aa, synthetic construct
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCAREDDSSGYYQDWGQGTLVTVSS

SEQ ID NO: 10 - protein, 25 aa, synthetic construct [sequence omitted]

SEQ ID NO: 11 - protein, 14 aa, synthetic construct
WVRQAPGQGLEWMG

SEQ ID NO: 12 - protein, 32 aa, synthetic construct
RVTITADKSTSTAYMELSSLRSEDTAVYYCAR

SEQ ID NO: 13 - protein, 11 aa, synthetic construct
WGQGTLVTVSS

SEQ ID NO: 14 - protein, 213 aa, synthetic construct
EIVMTQSPATLSLSPGESATLSCRAGQSINSHLAWYQQKPGQSPSLLIYDASTRATGVPARFSGSGFGTEFTLTISSLQSEDFAVYFCQQYEYWYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 11 aa, synthetic construct
RAGQSINSHLA

SEQ ID NO: 16 - protein, 7 aa, synthetic construct
DASTRAT

SEQ ID NO: 17 - protein, 8 aa, synthetic construct
QQYEYWYT

SEQ ID NO: 18 - protein, 107 aa, synthetic construct [sequence omitted]

SEQ ID NO: 19 - protein, 106 aa, synthetic construct
EIVMTQSPATLSLSPGESATLSCRAGQSINSHLAWYQQKPGQSPSLLIYDASTRATGVPARFSGSGFGTEFTLTISSLQSEDFAVYFCQQYEYWYTFGQGTKLEIK

SEQ ID NO: 20 - protein, 23 aa, synthetic construct
EIVMTQSPATLSLSPGESATLSC

SEQ ID NO: 21 - protein, 15 aa, synthetic construct
WYQQKPGQSPSLLIY

SEQ ID NO: 22 - protein, 32 aa, synthetic construct
GVPARFSGSGFGTEFTLTISSLQSEDFAVYFC

SEQ ID NO: 23 - protein, 10 aa, synthetic construct
FGQGTKLEIK

SEQ ID NO: 24 - protein, 213 aa, synthetic construct
EIVMTQSPATLSVSPGERATLSCRAGQSINSHLAWYQQKPGQAPRLLIYDASTRATGIPARFSGSGSGTEFTLTISSLQSEDFAVYYCQQYEYWYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 106 aa, synthetic construct
EIVMTQSPATLSVSPGERATLSCRAGQSINSHLAWYQQKPGQAPRLLIYDASTRATGIPARFSGSGSGTEFTLTISSLQSEDFAVYYCQQYEYWYTFGQGTKLEIK

SEQ ID NO: 26 - protein, 23 aa, synthetic construct
EIVMTQSPATLSVSPGERATLSC

SEQ ID NO: 27 - protein, 15 aa, synthetic construct
WYQQKPGQAPRLLIY

SEQ ID NO: 28 - protein, 32 aa, synthetic construct
GIPARFSGSGSGTEFTLTISSLQSEDFAVYYC

SEQ ID NO: 29 - protein, 449 aa, synthetic construct
QVQLVQSGAEVKKPGSSVKVSCKASGGTFSTYAISWVRQAPGQGLEWMGRIIPILGIANYAQKFQGRVTITADKSTSTAYMELSSLRSEDTAVYYCAREDDSSGYYQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 10 aa, synthetic construct
GGTFSTYAIS

SEQ ID NO: 31 - protein, 119 aa, synthetic construct
QVQLVQSGAEVKKPGSSVKVSCKASGGTFSTYAISWVRQAPGQGLEWMGRIIPILGIANYAQKFQGRVTITADKSTSTAYMELSSLRSEDTAVYYCAREDDSSGYYQDWGQGTLVTVSS

SEQ ID NO: 32 - protein, 213 aa, synthetic construct
EIVMTQSPATLSVSPGERATLSCRAGQSINSYLAWYQQKPGQAPRLLIYDASTRATGIPARFSGSGSGTEFTLTISSLQSEDFAVYYCQQYEYWYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 11 aa, synthetic construct
RAGQSINSYLA

SEQ ID NO: 34 - protein, 106 aa, synthetic construct
EIVMTQSPATLSVSPGERATLSCRAGQSINSYLAWYQQKPGQAPRLLIYDASTRATGIPARFSGSGSGTEFTLTISSLQSEDFAVYYCQQYEYWYTFGQGTKLEIK

SEQ ID NO: 35 - protein, 449 aa, synthetic construct
QVQLVQSGAEVKKPGSSVKVSCKASGGELQDLAISWVRQAPGQGLEWMGRIIPILGIANYAQKFQGRVTITADKSTSTAYMELSSLRSEDTAVYYCAREDDISGYYQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 10 aa, synthetic construct
GGELQDLAIS

SEQ ID NO: 37 - protein, 10 aa, synthetic construct
EDDISGYYQD

SEQ ID NO: 38 - protein, 119 aa, synthetic construct
QVQLVQSGAEVKKPGSSVKVSCKASGGELQDLAISWVRQAPGQGLEWMGRIIPILGIANYAQKFQGRVTITADKSTSTAYMELSSLRSEDTAVYYCAREDDISGYYQDWGQGTLVTVSS

SEQ ID NO: 39 - protein, 448 aa, synthetic construct
QVQLVQSGAEVKKPGSSVKVSCKASGGTFSTYAISWVRQAPGQGLEWMGRIIPILGIANYAQKFQGRVTITADKSTSTAYMELSSLRSEDTAVYYCAREDDSSGYYQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 106 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 448 aa, synthetic construct
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCAREDDSSGYYQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 448 aa, synthetic construct
QVQLVQSGAEVKKPGSSVKVSCKASGGELQDLAISWVRQAPGQGLEWMGRIIPILGIANYAQKFQGRVTITADKSTSTAYMELSSLRSEDTAVYYCAREDDISGYYQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 448 aa, synthetic construct
QVQLVQSGAEVKKPGSSVKVSCKASGGELQTLAISWVRQAPGQGLEWMGRIIPILGIANYAQKFQGRVTITADKSTSTAYMELSSLRSEDTAVYYCAREDDISGYYQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 10 aa, synthetic construct
GGELQTLAIS

SEQ ID NO: 45 - protein, 119 aa, synthetic construct
QVQLVQSGAEVKKPGSSVKVSCKASGGELQTLAISWVRQAPGQGLEWMGRIIPILGIANYAQKFQGRVTITADKSTSTAYMELSSLRSEDTAVYYCAREDDISGYYQDWGQGTLVTVSS

SEQ ID NO: 46 - protein, 448 aa, synthetic construct
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCAREDDISGYYQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 119 aa, synthetic construct
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCAREDDISGYYQDWGQGTLVTVSS

SEQ ID NO: 48 - protein, 448 aa, synthetic construct
QVQLVQSGAEVKKPGSSVKVSCKASGGTFSTYAISWVRQAPGQGLEWMGRIIPILGIANYAQKFQGRVTITADKSTSTAYMELSSLRSEDTAVYYCAREDDISGYYQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 119 aa, synthetic construct
QVQLVQSGAEVKKPGSSVKVSCKASGGTFSTYAISWVRQAPGQGLEWMGRIIPILGIANYAQKFQGRVTITADKSTSTAYMELSSLRSEDTAVYYCAREDDISGYYQDWGQGTLVTVSS

SEQ ID NO: 50 - protein, 448 aa, synthetic construct
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCAREEDISGYYQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 10 aa, synthetic construct
EEDISGYYQD

SEQ ID NO: 52 - protein, 119 aa, synthetic construct
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCAREEDISGYYQDWGQGTLVTVSS

SEQ ID NO: 53 - protein, 213 aa, synthetic construct
DIVMTQSPATLSVSPGERATLSCRAGQSISSYLAWYQQKPGQAPRLLIYDASTRATGIPARFSGSGSGTEFTLTISSLQSEDFAVYYCQQYEYWYSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 11 aa, synthetic construct
RAGQSISSYLA

SEQ ID NO: 55 - protein, 8 aa, synthetic construct
QQYEYWYS

SEQ ID NO: 56 - protein, 106 aa, synthetic construct
DIVMTQSPATLSVSPGERATLSCRAGQSISSYLAWYQQKPGQAPRLLIYDASTRATGIPARFSGSGSGTEFTLTISSLQSEDFAVYYCQQYEYWYSFGQGTKLEIK

SEQ ID NO: 57 - protein, 23 aa, synthetic construct
DIVMTQSPATLSVSPGERATLSC

SEQ ID NO: 58 - protein, 213 aa, synthetic construct
EIVMTQSPATLSVSPGERATLSCRAGQKISSYLAWYQQKPGQAPRLLIYDASTRATGIPARFSGSGSGTEFTLTISSLQSEDFAVYYCQQYEYWYSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 11 aa, synthetic construct
RAGQKISSYLA

SEQ ID NO: 60 - protein, 106 aa, synthetic construct
EIVMTQSPATLSVSPGERATLSCRAGQKISSYLAWYQQKPGQAPRLLIYDASTRATGIPARFSGSGSGTEFTLTISSLQSEDFAVYYCQQYEYWYSFGQGTKLEIK

SEQ ID NO: 61 - protein, 213 aa, synthetic construct
EIVMTQSPATLSVSPGERATLSCRAGQSISHYLAWYQQKPGQAPRLLIYDASTRATGIPARFSGSGSGTEFTLTISSLQSEDFAVYYCQQYEYWYSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 11 aa, synthetic construct
RAGQSISHYLA

SEQ ID NO: 63 - protein, 106 aa, synthetic construct
EIVMTQSPATLSVSPGERATLSCRAGQSISHYLAWYQQKPGQAPRLLIYDASTRATGIPARFSGSGSGTEFTLTISSLQSEDFAVYYCQQYEYWYSFGQGTKLEIK

SEQ ID NO: 64 - protein, 213 aa, synthetic construct
EIVMTQSPATLSVSPGERATLSCRAGQSISRYLAWYQQKPGQAPRLLIYDASTRATGIPARFSGSGSGTEFTLTISSLQSEDFAVYYCQQYEYWYSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 11 aa, synthetic construct
RAGQSISRYLA

SEQ ID NO: 66 - protein, 106 aa, synthetic construct
EIVMTQSPATLSVSPGERATLSCRAGQSISRYLAWYQQKPGQAPRLLIYDASTRATGIPARFSGSGSGTEFTLTISSLQSEDFAVYYCQQYEYWYSFGQGTKLEIK

SEQ ID NO: 67 - protein, 213 aa, synthetic construct
DIVMTQSPATLSVSPGERATLSCRAGQSISSYLAWYQQKPGQAPRLLIYDASSRATGIPARFSGSGSGTEFTLTISSLQSEDFAVYYCQQYEYWYSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 7 aa, synthetic construct
DASSRAT

SEQ ID NO: 69 - protein, 106 aa, synthetic construct
DIVMTQSPATLSVSPGERATLSCRAGQSISSYLAWYQQKPGQAPRLLIYDASSRATGIPARFSGSGSGTEFTLTISSLQSEDFAVYYCQQYEYWYSFGQGTKLEIK

SEQ ID NO: 70 - protein, 213 aa, synthetic construct
EIVMTQSPATLSVSPGERATLSCRAGQKISSYLAWYQQKPGQAPRLLIYDASSRATGIPARFSGSGSGTEFTLTISSLQSEDFAVYYCQQYEYWYSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 106 aa, synthetic construct
EIVMTQSPATLSVSPGERATLSCRAGQKISSYLAWYQQKPGQAPRLLIYDASSRATGIPARFSGSGSGTEFTLTISSLQSEDFAVYYCQQYEYWYSFGQGTKLEIK

SEQ ID NO: 72 - protein, 213 aa, synthetic construct
EIVMTQSPATLSVSPGERATLSCRAGQSISHYLAWYQQKPGQAPRLLIYDASSRATGIPARFSGSGSGTEFTLTISSLQSEDFAVYYCQQYEYWYSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 106 aa, synthetic construct
EIVMTQSPATLSVSPGERATLSCRAGQSISHYLAWYQQKPGQAPRLLIYDASSRATGIPARFSGSGSGTEFTLTISSLQSEDFAVYYCQQYEYWYSFGQGTKLEIK

SEQ ID NO: 74 - protein, 213 aa, synthetic construct
EIVMTQSPATLSVSPGERATLSCRAGQSISRYLAWYQQKPGQAPRLLIYDASSRATGIPARFSGSGSGTEFTLTISSLQSEDFAVYYCQQYEYWYSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 106 aa, synthetic construct
EIVMTQSPATLSVSPGERATLSCRAGQSISRYLAWYQQKPGQAPRLLIYDASSRATGIPARFSGSGSGTEFTLTISSLQSEDFAVYYCQQYEYWYSFGQGTKLEIK

SEQ ID NO: 76 - protein, 448 aa, synthetic construct
QVQLVQSGAEVKKPGSSVKVSCKASGGELQTLAISWVRQAPGQGLEWMGRIIPILGIANYAQKFQGRVTITADKSTSTAYMELSSLRSEDTAVYYCAREEDISGYYQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 119 aa, synthetic construct
QVQLVQSGAEVKKPGSSVKVSCKASGGELQTLAISWVRQAPGQGLEWMGRIIPILGIANYAQKFQGRVTITADKSTSTAYMELSSLRSEDTAVYYCAREEDISGYYQDWGQGTLVTVSS

SEQ ID NO: 78 - protein, 213 aa, synthetic construct
EIVMTQSPATLSVSPGERATLSCRAGQSISSYLAWYQQKPGQAPRLLIYDASSRATGIPARFSGSGSGTEFTLTISSLQSEDFAVYYCQQYEYWYSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 106 aa, synthetic construct
EIVMTQSPATLSVSPGERATLSCRAGQSISSYLAWYQQKPGQAPRLLIYDASSRATGIPARFSGSGSGTEFTLTISSLQSEDFAVYYCQQYEYWYSFGQGTKLEIK

SEQ ID NO: 80 - protein, 448 aa, synthetic construct
QVQLVQSGAEVKKPGSSVKVSCKASGGELQTYAISWVRQAPGQGLEWMGRIIPILGIANYAQKFQGRVTITADKSTSTAYMELSSLRSEDTAVYYCAREEDISGYYQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 81 - protein, 10 aa, synthetic construct
GGELQTYAIS

SEQ ID NO: 82 - protein, 119 aa, synthetic construct
QVQLVQSGAEVKKPGSSVKVSCKASGGELQTYAISWVRQAPGQGLEWMGRIIPILGIANYAQKFQGRVTITADKSTSTAYMELSSLRSEDTAVYYCAREEDISGYYQDWGQGTLVTVSS

SEQ ID NO: 83 - protein, 448 aa, synthetic construct
QVQLVQSGAEVKKPGSSVKVSCKASGGELSTYAISWVRQAPGQGLEWMGRIIPILGIANYAQKFQGRVTITADKSTSTAYMELSSLRSEDTAVYYCAREEDISGYYQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 84 - protein, 10 aa, synthetic construct
GGELSTYAIS

SEQ ID NO: 85 - protein, 119 aa, synthetic construct
QVQLVQSGAEVKKPGSSVKVSCKASGGELSTYAISWVRQAPGQGLEWMGRIIPILGIANYAQKFQGRVTITADKSTSTAYMELSSLRSEDTAVYYCAREEDISGYYQDWGQGTLVTVSS

SEQ ID NO: 86 - protein, 448 aa, synthetic construct
QVQLVQSGAEVKKPGSSVKVSCKASGGEFQSYAISWVRQAPGQGLEWMGRIIPILGIANYAQKFQGRVTITADKSTSTAYMELSSLRSEDTAVYYCAREEDISGYYQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 87 - protein, 10 aa, synthetic construct
GGEFQSYAIS

SEQ ID NO: 88 - protein, 119 aa, synthetic construct
QVQLVQSGAEVKKPGSSVKVSCKASGGEFQSYAISWVRQAPGQGLEWMGRIIPILGIANYAQKFQGRVTITADKSTSTAYMELSSLRSEDTAVYYCAREEDISGYYQDWGQGTLVTVSS

SEQ ID NO: 89 - protein, 448 aa, synthetic construct
QVQLVQSGAEVKKPGSSVKVSCKASGGTFQTYAISWVRQAPGQGLEWMGRIIPILGIANYAQKFQGRVTITADKSTSTAYMELSSLRSEDTAVYYCAREEDISGYYQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 10 aa, synthetic construct
GGTFQTYAIS

SEQ ID NO: 91 - protein, 119 aa, synthetic construct
QVQLVQSGAEVKKPGSSVKVSCKASGGTFQTYAISWVRQAPGQGLEWMGRIIPILGIANYAQKFQGRVTITADKSTSTAYMELSSLRSEDTAVYYCAREEDISGYYQDWGQGTLVTVSS

SEQ ID NO: 92 - protein, 448 aa, synthetic construct
QVQLVQSGAEVKKPGSSVKVSCKASGGTLSTYAISWVRQAPGQGLEWMGRIIPILGIANYAQKFQGRVTITADKSTSTAYMELSSLRSEDTAVYYCAREEDISGYYQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 93 - protein, 10 aa, synthetic construct
GGTLSTYAIS

SEQ ID NO: 94 - protein, 119 aa, synthetic construct
QVQLVQSGAEVKKPGSSVKVSCKASGGTLSTYAISWVRQAPGQGLEWMGRIIPILGIANYAQKFQGRVTITADKSTSTAYMELSSLRSEDTAVYYCAREEDISGYYQDWGQGTLVTVSS

SEQ ID NO: 95 - protein, 448 aa, synthetic construct
QVQLVQSGAEVKKPGSSVKVSCKASGGELSSYAISWVRQAPGQGLEWMGRIIPILGIANYAQKFQGRVTITADKSTSTAYMELSSLRSEDTAVYYCAREEDISGYYQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 10 aa, synthetic construct
GGELSSYAIS

SEQ ID NO: 97 - protein, 119 aa, synthetic construct
QVQLVQSGAEVKKPGSSVKVSCKASGGELSSYAISWVRQAPGQGLEWMGRIIPILGIANYAQKFQGRVTITADKSTSTAYMELSSLRSEDTAVYYCAREEDISGYYQDWGQGTLVTVSS

SEQ ID NO: 98 - protein, 448 aa, synthetic construct
QVQLVQSGAEVKKPGSSVKVSCKASGGEFSTYAISWVRQAPGQGLEWMGRIIPILGIANYAQKFQGRVTITADKSTSTAYMELSSLRSEDTAVYYCAREEDISGYYQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 10 aa, synthetic construct
GGEFSTYAIS

SEQ ID NO: 100 - protein, 119 aa, synthetic construct
QVQLVQSGAEVKKPGSSVKVSCKASGGEFSTYAISWVRQAPGQGLEWMGRIIPILGIANYAQKFQGRVTITADKSTSTAYMELSSLRSEDTAVYYCAREEDISGYYQDWGQGTLVTVSS

SEQ ID NO: 101 - protein, 449 aa, synthetic construct
QVQLVQSGAEVKKPGSSVKVSCKASGGELSSYAISWVRQAPGQGLEWMGRIIPILGIANYAQKFQGRVTITADKSTSTAYMELSSLRSEDTAVYYCAREEDISGYYQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 10 aa, synthetic construct
  VARIANT 3: note = Threonine or Glutamic acid
  VARIANT 4: note = Phenylalanine or Leucine
  VARIANT 5: note = Serine or Glutamine
  VARIANT 6: note = Threonine or Aspartic acid or Serine
  VARIANT 7: note = Tyrosine or Leucine
GGXXXXXAIS

SEQ ID NO: 103 - protein, 10 aa, synthetic construct
  VARIANT 2: note = Aspartic acid or Glutamic acid
  VARIANT 4: note = Serine or Isoleucine
EXDXSGYYQD

SEQ ID NO: 104 - protein, 11 aa, synthetic construct
  VARIANT 5: note = Serine or Lysine
  VARIANT 7: note = Asparagine or Serine
  VARIANT 8: note = Serine or Histidine or Arginine
RAGQXIXXYLA

SEQ ID NO: 105 - protein, 7 aa, synthetic construct
  VARIANT 4: note = Serine or Threonine
DASXRAT

SEQ ID NO: 106 - protein, 8 aa, synthetic construct
  VARIANT 8: note = Serine or Threonine
QQYEYWYX

SEQ ID NO: 107 - protein, 119 aa, synthetic construct; identical to SEQ ID NO: 9

SEQ ID NO: 108 - protein, 119 aa, synthetic construct
QVQLVQSGAEVKKPGSSVKVSCKASGGELQDLAISWVRQAPGQGLEWMGRIIPILGIANYAQKFQGRVTITADKSTSTAYMELSSLRSEDTAVYYCAREDDSSGYYQDWGQGTLVTVSS

SEQ ID NO: 109 - protein, 119 aa, synthetic construct; identical to SEQ ID NO: 31

SEQ ID NO: 110 - protein, 119 aa, synthetic construct
QVQLVQSGAEVKKPGSSVKVSCKASGGELQTLAISWVRQAPGQGLEWMGRIIPILGIANYAQKFQGRVTITADKSTSTAYMELSSLRSEDTAVYYCAREDDSSGYYQDWGQGTLVTVSS

SEQ ID NO: 111 - protein, 107 aa, synthetic construct
EIVMTCSPATLSVSPGERATLSCRAGCSINSHLAWYCQKPGQAPRILLIYDASTRATGIPARFSGSGSGTEFTLTISSLCSEDFAVYYCQCYEYWYTFGCGTKLEIK

SEQ ID NO: 112 - protein, 107 aa, synthetic construct
EIVMTCSPATLSLSPGESATLSCRAGCSINSHLAWYCQKPGQSPSILLIYDASTRATGVPARFSGSGFGTEFTLTISSLCSEDFAVYFCQCYEYWYTFGCGTKLEIK

SEQ ID NO: 113 - protein, 107 aa, synthetic construct
EIVMTCSPATLSVSPGERATLSCRAGCSINSYLAWYCQKPGQAPRILLIYDASTRATGIPARFSGSGSGTEFTLTISSLCSEDFAVYYCQCYEYWYTFGCGTKLEIK

SEQ ID NO: 114 - protein, 119 aa, synthetic construct
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCAREEDSSGYYQDWGQGTLVTVSS

SEQ ID NO: 115 - protein, 107 aa, synthetic construct
EIVMTCSPATLSVSPGERATLSCRAGCKISSYLAWYCQKPGQAPRILLIYDASTRATGIPARFSGSGSGTEFTLTISSLCSEDFAVYYCQCYEYWYSFGCGTKLEIK

SEQ ID NO: 116 - protein, 107 aa, synthetic construct
EIVMTCSPATLSVSPGERATLSCRAGCSISHYLAWYCQKPGQAPRILLIYDASTRATGIPARFSGSGSGTEFTLTISSLCSEDFAVYYCQCYEYWYSFGCGTKLEIK

SEQ ID NO: 117 - protein, 107 aa, synthetic construct
EIVMTCSPATLSVSPGERATLSCRAGCSISRYLAWYCQKPGQAPRILLIYDASTRATGIPARFSGSGSGTEFTLTISSLCSEDFAVYYCQCYEYWYSFGCGTKLEIK

SEQ ID NO: 118 - protein, 107 aa, synthetic construct
EIVMTCSPATLSVSPGERATLSCRAGCKISSYLAWYCQKPGQAPRILLIYDASSRATGIPARFSGSGSGTEFTLTISSLCSEDFAVYYCQCYEYWYSFGCGTKLEIK

SEQ ID NO: 119 - protein, 107 aa, synthetic construct
EIVMTCSPATLSVSPGERATLSCRAGCSISHYLAWYCQKPGQAPRILLIYDASSRATGIPARFSGSGSGTEFTLTISSLCSEDFAVYYCQCYEYWYSFGCGTKLEIK

SEQ ID NO: 120 - protein, 107 aa, synthetic construct
EIVMTCSPATLSVSPGERATLSCRAGCSISRYLAWYCQKPGQAPRILLIYDASSRATGIPARFSGSGSGTEFTLTISSLCSEDFAVYYCQCYEYWYSFGCGTKLEIK

SEQ ID NO: 121 - protein, 119 aa, synthetic construct
QVQLVQSGAEVKKPGSSVKVSCKASGGELSTYAISWVRQAPGQGLEWMGRIIPILGIANYAQKFQGRVTITADKSTSTAYMELSSLRSEDTAVYYCAREDDSSGYYQDWGQGTLVTVSS

SEQ ID NO: 122 - protein, 119 aa, synthetic construct
QVQLVQSGAEVKKPGSSVKVSCKASGGELQTYAISWVRQAPGQGLEWMGRIIPILGIANYAQKFQGRVTITADKSTSTAYMELSSLRSEDTAVYYCAREDDSSGYYQDWGQGTLVTVSS

SEQ ID NO: 123 - protein, 119 aa, synthetic construct
QVQLVQSGAEVKKPGSSVKVSCKASGGEFQSYAISWVRQAPGQGLEWMGRIIPILGIANYAQKFQGRVTITADKSTSTAYMELSSLRSEDTAVYYCAREDDSSGYYQDWGQGTLVTVSS

SEQ ID NO: 124 - protein, 119 aa, synthetic construct
QVQLVQSGAEVKKPGSSVKVSCKASGGTFQTYAISWVRQAPGQGLEWMGRIIPILGIANYAQKFQGRVTITADKSTSTAYMELSSLRSEDTAVYYCAREDDSSGYYQDWGQGTLVTVSS

SEQ ID NO: 125 - protein, 119 aa, synthetic construct
QVQLVQSGAEVKKPGSSVKVSCKASGGTLSTYAISWVRQAPGQGLEWMGRIIPILGIANYAQKFQGRVTITADKSTSTAYMELSSLRSEDTAVYYCAREDDSSGYYQDWGQGTLVTVSS

SEQ ID NO: 126 - protein, 119 aa, synthetic construct
QVQLVQSGAEVKKPGSSVKVSCKASGGELSSYAISWVRQAPGQGLEWMGRIIPILGIANYAQKFQGRVTITADKSTSTAYMELSSLRSEDTAVYYCAREDDSSGYYQDWGQGTLVTVSS

SEQ ID NO: 127 - protein, 119 aa, synthetic construct
QVQLVQSGAEVKKPGSSVKVSCKASGGEFSTYAISWVRQAPGQGLEWMGRIIPILGIANYAQKFQGRVTITADKSTSTAYMELSSLRSEDTAVYYCAREDDSSGYYQDWGQGTLVTVSS

SEQ ID NO: 128 - protein, 107 aa, synthetic construct
EIVMTCSPATLSVSPGERATLSCRAGCSISSYLAWYCQKPGQAPRILLIYDASSRATGIPARFSGSGSGTEFTLTISSLCSEDFAVYYCQCYEYWYSFGCGTKLEIK

SEQ ID NO: 129 - protein, 339 aa, synthetic construct
MLDAERLKHLIVTPSGAGEQNMIGMTPTVIAVHYLDETEQWEKFGLEKRQGALELIKKFVKRAPSTWLTAYVVKVFSLAVNLIAIDSQVLCGAVKWLILEKQKPDGVFQEDAPVIHTAFVLISLQEAKDICEEQVNSLPGSITKAGDFLEANYMNLQRSYTVAIAGYALAQMGFRWEDPGKQLYNVEATSYALLALLQLKDFDFVPPVVRWLNEQRYYGGGYGSTQATFMVFPPPILNGRISYYSTPIAVGTVIRYSCSGTFRLIGEKSLLCITKDKVDGTWDKPAPKCEKIRGSTPYRHGDSVTFACKTNFSMNGNKSVWCQANNMWGPTRLPTCVSV

SEQ ID NO: 130 - protein, 339 aa, synthetic construct
AVDAERLKHLIVTPSGCGEQNMIGMTPTVIAVHYLDETEQWEKFGLEKRQGALELIKKFVKRAPSTWLTAYVVKVFSLAVNLIAIDSQVLCGAVKWLILEKQKPDGVFQEDAPVIHTAFVLISLQEAKDICEEQVNSLPGSITKAGDFLEANYMNLQRSYTVAIAGYALAQMGPRWEDPGKQLYNVEATSYALLALLQLKDFDFVPPVVRWLNEQRYYGGGYGSTQATFMVFPPPILNGRISYYSTPIAVGTVIRYSCSGTFRLIGEKSLLCITKDKVDGTWDKPAPKCEKIRGSTPYRHGDSVTFACKTNFSMNGNKSVWCQANNMWGPTRLPTCVSV

SEQ ID NO: 131 - protein, 340 aa, synthetic construct
AIDAERLKHLIVTPSGCGEQNMITMTPTVIAVHYLDETEQWEKFGPEKRQGALELIKKFLNRAPSTWLTAYVVKVFSLAVNLIAIDSQVLCGAVKWLILEKQKPDGVFQEDAPVIHTAFVLISLQEAKEICEEQVNSLPGSITKAGDFLEANYMNLQRSYTVAIAAYALAQMGPRWEEPGQQLYNVEATSYALLALLQLKDFDFVPPVVRWLNEQRYYGGGYGSTQATFMVFPPPILNGRISHYSTPITLGTVIRYICSNGFRLIGEKNIICITKDKVVGIWDKPAPKCEKTRGAEPPYIHGDFVTFACKTNFSMNGNKSVWCQANSKWGPTRLPTCESV

SEQ ID NO: 132 - protein, 339 aa, synthetic construct
AVDGERLKHLIVTPSGCGEQNMIGMTPTVIAVHYLDQTEQWEKFGLEKRQEALELIKKFNNRPPSTWLTAYVVKVFSLAANLIAIDSQVLCGAVKWLILEKQKPDGVFQEDGPVIHTAFVLIALQEARDICEGQVNSLPGSINKAGEYLEASYLNLQRPYTVAIAGYALALMNKRWEEPGQQLYNVEATSYALLALLLLKDFDSVPPVVRWLNEQRYYGGGYGSTQATFMVFPPEVNNTRRLYYSLPVLPGTVVRYTCLPGYRLIGEKAIFCISKDQVTATWDKATPICEINPGSRPPFKHGDSVTVTCKANFTMKGSKTVWCRANKMWGPTPLPVCES

SEQ ID NO: 133 - protein, 340 aa, synthetic construct
AVDGERLKHLIVTPAGCGEQNMIGMTPTVIAVHYLDQTEQWEKFGIEKKRQEALELIKKFNNRPPSTWLTAYVVKVFSLAANLIAIDSHVLCGAVKWLILEKQKPDGVFQEDGPVIHTAFVLIALQEARDICEGQVNSLPGSINKAGEYIEASYMNLQRPYTVAIAGYALALMNKRWEEPDQQLYNVEATSYALLALLLLKDFDSVPPVVRWLNEQRYYGGGYGSTQATFMVFPPEVKNARKPYYSLPIVPGTVLRYTCSPSYRLIGEKAIFCISENQVHATWDKAPPICEMNKGSKAPFRHGDSVTFTCKANFTMKGSKTVWCQANEMWGPTALPVCES

SEQ ID NO: 134 - protein, 340 aa, synthetic construct
AIDGERLKHLIQTPSGCGEQNMIGMTPTVIAVHYLDSTDQWEKFGLEKRQESLELIRKFQYRPPSTWLTAYVVKVFALAANLIAIDSKDLCETVKWLILEKQKPDGIFQEDGPVIHTAFVLIALHEAKDICEAQVNSLGRSIAKAGDFLENHYRELRRPYTVAIAAYALLALLGKRWEEPNQKLYNVEATSYALLALLARKDYDTTPPVVRWLNEQRYYGGGYGSTQATFMVFPPSIKNGRSSYYSGPVAHNAVVTYTCQSAFRLIGERRLFCISKDNVNGIWDKAPPICERSKTSRPPFRHGDAVTFSCNANFTMKGNKTVWCRANRRWGPTPLPTCES